(19) United States
(12) Patent Application Publication
(10) Pub. No.: US 2018/0133211 A1
(43) Pub. Date: May 17, 2018

(54) METHODS AND MATERIALS FOR PROMOTING BONE FORMATION

(71) Applicant: Mayo Foundation for Medical Education and Research, Rochester, MN (US)

(72) Inventors: Andre J. van Wijnen, Rochester, MN (US); Scott M. Riester, Rochester, MN (US); Emily T. Camilleri, Rochester, MN (US); Amel Dudakovic, Rochester, MN (US); Roman Thaler, Rochester, MN (US); John W. Sperling, Rochester, MN (US)

(73) Assignee: Mayo Foundation for Medical Education and Research, Rochester, MN (US)

(21) Appl. No.: 15/574,677

(22) PCT Filed: May 19, 2016
(86) PCT No.: PCT/US2016/033211

§ 371 Date: Nov. 16, 2017

Related U.S. Application Data
(60) Provisional application No. 62/163,540, filed on May 19, 2015.
(60) Provisional application No. 62/233,531, filed on Sep. 28, 2015.

(51) Int. Cl. A61K 31/496; A61P 19/10; A61P 19/08; A61K 31/5377; A61K 31/4439; A61K 31/26; A61K 31/385; A61K 31/095

(52) U.S. Cl. CPC A61K 31/496; A61P 19/10; A61P 19/08; A61K 31/095; A61K 31/4439; A61K 31/26; A61K 31/385; A61K 31/5377

(57) ABSTRACT

This document provides methods and materials involved in promoting new bone formation for the treatment of medical conditions such as osteoporosis, bone defects, bone injury (e.g., fractures) implant ingrowth, and joint/spine fusions. For example, methods and materials for using sulforaphane and/or EZH2 polypeptide inhibitors (e.g., GSK126 or UNC1999) to treat osteoporosis, bone fractures and defects, implant ingrowth, and joint fusions are provided.

### CROSS-REFERENCE TO RELATED APPLICATIONS

This application claims priority from U.S. Provisional Application Ser. No. 62/163,540, filed May 19, 2015, and U.S. Provisional Application Ser. No. 62/233,531, filed Sep. 28, 2015.

### BACKGROUND

### 1. Technical Field

This document relates to methods and materials involved in treating osteoporosis. For example, this document provides methods and materials for using sulforaphane and/or inhibitors of a histone methyl transferase Enhancer-of-Zeste homolog 2 (EZH2) polypeptide to promote and/or accelerate bone formation for treating conditions such as osteoporosis, fracture healing, bone defects, implant ingrowth, joint and spine fusion, and other conditions that require new bone formation.

### 2. Background Information

Decreased bone mass density (BMD) is associated with increased fracture risk and an imbalance in the biological activities of bone-forming osteoblasts and bone-resorbing osteoclasts (Consensus development conference, Am. J. Med., 94:646-650 (1993); Burge et al., J. Bone Miner. Res., 22:465-475 (2007); and Dennison and Cooper, Horm. Res., 54(Suppl 1):58-63 (2000)). Loss of BMD observed in individuals with osteoporosis, a very prevalent skeletal disease, can be mitigated by anti-resorptive strategies including treatments with bisphosphonates, selective estrogen receptor modulators (e.g., raloxifene), or antibodies that inactivate the osteoclast-stimulatory specific ligand RANKL (Denosumab). Therapeutics for osteoporosis that can stimulate bone formation include bone morphogenetic proteins (e.g., BMP2), intermittent treatment with parathyroid hormone (PTH) or PTH related protein, and antibody that suppress WNT inhibitors (e.g., SOST).

### SUMMARY

This document provides methods and materials for enhancing bone regeneration. The methods provided herein can be applied to treat medical conditions such as osteoporosis, fracture or other bone injury, joint fusion (including spine fusion), implant ingrowth, bone defects, and other conditions in which formation of new bone is desired or required. In some embodiments, this document provides methods and materials for using sulforaphane and/or EZH2 polypeptide inhibitors (e.g., GSK126 or UNC1999) to treat osteoporosis, to facilitate healing of fractures, or to promote ingrowth at bone fusion sites, thus improving clinical outcomes.

As described herein, for example, administering an EZH2 polypeptide inhibitor such as GSK126 or UNC1999 to a mammal with osteoporosis can promote bone formation, improve cortical bone structure, and/or reverse osteoporosis, and administering sulforaphane to a mammal can epigenetically stimulate osteoblast activity and diminish osteoclast bone resorption. In some cases, sulforaphane and/or an EZH2 polypeptide inhibitor can be administered to a mammal (e.g., a human) suspected of developing osteoporosis in a manner that slows the progression of osteoporosis or prevents the onset of osteoporosis. Having the ability to promote bone formation, improve cortical bone structure, reverse osteoporosis, slow the progression of osteoporosis, or prevent the onset of osteoporosis as described herein can allow patients with osteoporosis or patients at risk of osteoporosis to experience happier and healthier lives.

In general, one aspect of this document features a method for reversing osteoporosis in a mammal. The method comprises, or consists essentially of, (a) identifying a mammal as having osteoporosis, and (b) administering an inhibitor of an EZH2 polypeptide to the mammal, thereby reversing the osteoporosis. The mammal can be a human. The inhibitor can be selected from the group consisting of GSK126, UNC1999, EPZ005687, GSK343, EPZ-6438, and EI1.

In another aspect, this document features a method for preventing the onset of osteoporosis in a mammal at risk for developing osteoporosis. The method comprises, or consists essentially of, (a) identifying a mammal as being at risk for developing osteoporosis, and (b) administering an inhibitor of an EZH2 polypeptide to the mammal, thereby preventing the onset of osteoporosis. The mammal can be a human. The inhibitor can be selected from the group consisting of GSK126, UNC1999, EPZ005687, GSK343, EPZ-6438, and EI1.

In another aspect, this document features a method for treating a bone injury or bone defect in a mammal. The method comprises, or consists essentially of, (a) identifying a mammal as having a bone injury or defect, and (b) administering an inhibitor of an EZH2 polypeptide to the mammal, thereby enhancing bone healing and repair. The mammal can be a human. The inhibitor can be selected from the group consisting of GSK126, UNC1999, EPZ005687, GSK343, EPZ-6438, and EI1. The bone injury can be a fracture.

In yet another aspect, this document features a method for enhancing joint fusion in a mammal. The method comprises, or consists essentially of, (a) identifying a mammal as being in need of a procedure to promote joint fusion, or as having undergone a procedure to promote joint fusion, and (b) administering an inhibitor of an EZH2 polypeptide to the mammal, thereby promoting bone formation and enhancing the rate and/or strength of joint fusion. The mammal can be a human. The inhibitor can be selected from the group consisting of GSK126, UNC1999, EPZ005687, GSK343, EPZ-6438, and EI1. The procedure can be spinal fusion.

In another aspect, this document features a method for enhancing implant ingrowth in a mammal. The method comprises, or consists essentially of, (a) identifying a mammal as being in need of, or as having, an implant that undergoes osteo-integration, and (b) administering an inhibitor of an EZH2 polypeptide to the mammal, thereby enhancing the rate and/or strength of implant osteo-integration. The mammal can be a human. The inhibitor can be selected from the group consisting of GSK126, UNC1999, EPZ005687, GSK343, EPZ-6438, and EI1. The implant can be a joint replacement.

In another aspect, this document features a method for reversing osteoporosis in a mammal. The method comprises, or consists essentially of, (a) identifying a mammal as having osteoporosis, and (b) administering (i) sulforaphane or a sulforaphane alternative and (ii) an inhibitor of an EZH2 polypeptide to the mammal, thereby reversing the osteoporosis. The mammal can be a human. The inhibitor can be selected from the group consisting of GSK126, UNC1999, EPZ005687, GSK343, EPZ-6438, and EI1. The method can comprise administering the sulforaphane. The method can comprise administering the sulforaphane alternative selected from the group consisting of sulforaphane, erucin, lipoic acid/α-lipoic acid/alpha lipoic acid/thioctic acid, and methylsulfonylmethane.

In another aspect, this document features a method for preventing the onset of osteoporosis in a mammal at risk for developing osteoporosis. The method comprises, or consists essentially of, (a) identifying a mammal as being at risk for developing osteoporosis, and (b) administering (i) sulforaphane or a sulforaphane alternative and (ii) an inhibitor of an EZH2 polypeptide to the mammal, thereby preventing the onset of osteoporosis. The mammal can be a human. The inhibitor can be selected from the group consisting of GSK126, UNC1999, EPZ005687, GSK343, EPZ-6438, and EI1. The method can comprise administering the sulforaphane. The method can comprise administering the sulforaphane alternative selected from the group consisting of sulforaphane, erucin, lipoic acid/α-lipoic acid/alpha lipoic acid/thioctic acid, and methylsulfonylmethane.

In still another aspect, this document features a method for treating a bone injury or bone defect in a mammal. The method comprises, or consists essentially of, (a) identifying a mammal as having a bone injury or defect, and (b) administering (i) sulforaphane or a sulforaphane alternative and (ii) an inhibitor of an EZH2 polypeptide to the mammal, thereby enhancing bone healing and repair. The mammal can be a human. The inhibitor can be selected from the group consisting of GSK126, UNC1999, EPZ005687, GSK343, EPZ-6438, and EI1. The method can include administering sulforaphane. The method can include administering a sulforaphane alternative selected from the group consisting of erucin, lipoic acid/α-lipoic acid/alpha lipoic acid/thioctic acid, and methylsulfonylmethane. The bone injury can be a fracture.

In another aspect, this document features a method for enhancing joint fusion in a mammal. The method comprises, or consists essentially of, (a) identifying a mammal as being in need of a procedure to promote joint fusion, or as having undergone a procedure to promote joint fusion, and (b) administering (i) sulforaphane or a sulforaphane alternative and (ii) an inhibitor of an EZH2 polypeptide to the mammal, thereby promoting bone formation and enhancing the rate and/or strength of joint fusion. The mammal can be a human. The inhibitor can be selected from the group consisting of GSK126, UNC1999, EPZ005687, GSK343, EPZ-6438, and EI1. The method can include administering sulforaphane. The method can include administering a sulforaphane alternative selected from the group consisting of erucin, lipoic acid/α-lipoic acid/alpha lipoic acid/thioctic acid, and methylsulfonylmethane. The procedure can be spinal fusion.

This document also features a method for enhancing implant ingrowth in a mammal. The method comprises, or consists essentially of, (a) identifying a mammal as being in need of, or as having, an implant that undergoes osteo-integration, and (b) administering (i) sulforaphane or a sulforaphane alternative and (ii) an inhibitor of an EZH2 polypeptide to the mammal, thereby enhancing the rate and/or strength of implant osteo-integration. The mammal can be a human. The inhibitor can be selected from the group consisting of GSK126, UNC1999, EPZ005687, GSK343, EPZ-6438, and EI1. The method can include administering sulforaphane. The method can include administering a sulforaphane alternative selected from the group consisting of erucin, lipoic acid/α-lipoic acid/alpha lipoic acid/thioctic acid, and methylsulfonylmethane. The implant can be a joint replacement.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and drawings, and from the claims.

### DESCRIPTION OF DRAWINGS

FIGS. 1A-1M provide evidence identifying EZH2 as an inhibitor of osteogenic differentiation. FIG. 1A: Epigenetic regulators exhibiting modulations in mRNA levels during osteogenic differentiation of clinical-grade MSCs were identified by expression screening using a semi-automated real-time RT-qPCR platform with >300 primer pairs. Sorted expression ratios for 322 mRNAs for epigenetic proteins were determined using differentiating cells for 11 days (D11) versus undifferentiated cells (D0). While >200 mRNAs are up-regulated, EZH2 is one of 14 genes that are down-regulated by more than 2 fold. FIGS. 1B and 1C: Levels of EZH2 and other representative mRNAs (alkaline phosphatase, ALPL; integrin-binding sialoprotein, IBSP; osteoprotegerin, OPG/TNFRSF11B; Twist homolog 2, TWIST2) were validated by mRNASeq from MSCs undergoing an osteogenic MSC time-course using standard conditions (plastic cell culture vessels) (FIG. 1B) or porous-sintered Titanium inserts (FIG. 1C); the latter is relevant for bone growth on orthopedic implants. Graphs show expression values (reads per kilobasepair per million mapped reads; RKPM) at different days of culture (D-1 is one day prior to osteogenic induction at D0). FIG. 1D: RT-qPCR (bar graph) and western blot analyses (insert) show down-regulation of, respectively, EZH2 mRNA and protein at different days of differentiation; β-actin controls for protein loading. FIG. 1E: diagram of the experimental protocol for treatment of MSCs with GSK126 (2 μM) shown in FIGS. 1F-1I. FIG. 1F: mRNA analysis by RT-qPCR of selected bone-related markers as indicated by the respective gene symbols (n=3). FIGS. 1G-1I: alkaline phosphatase staining (FIG. 1G) and quantitation (FIG. 1H) or alizarin-red staining (FIG. 10 for MSCs treated with vehicle or GSK126. FIG. 1J: Diagram of the experimental protocol for treatment of MSCs with siRNA for EZH2 shown in FIGS. 1I-1M; subconfluent cells (D-1) were transfected (TO at confluence (D0) and analyzed for RNA protein on the indicated days. FIGS. 1K-1M: RT-qPCR analysis of EZH2 mRNA (FIG. 1K), western blotting of EZH2 protein relative to β-actin (FIG. 1L), and RT-qPCR analysis of selected bone-related markers (FIG. 1M) for MSCs treated with control non-silencing RNA or EZH2 siRNA.

FIGS. 2A-2E show EZH2 Inhibition in MSCs. FIG. 2A: Epigenetic regulators suppressed during osteogenic differentiation of MSCs. Cells were differentiated in osteogenic medium for eleven days, RNA collected at indicated times, and real-time RT-qPCR was used to measure expression of ˜330 epigenetic regulators. Twelve genes (minimally expressed at 0.1 where GAPDH=100), including EZH2, suppressed during osteogenic differentiation and eight related genes with different expression patterns are shown (blue=relative low expression, red=relative high expression). FIG. 2B: Toxicity profile of GSK126 based on MTS activity in MSCs treated with different inhibitor concentration for 3 days (n=3). FIGS. 2C and 2D: Dose-dependent (FIG. 2C) and time-dependent (FIG. 2D) modulation of H3K27Me3 by GSK126 in MSCs. Cells were treated with different concentrations of GSK126 at 1 day after plating and harvested 3 days later for western blotting (FIG. 2C), or treated with 2 μM GSK126 and protein lysates collected at the specified times (FIG. 2D). FIG. 2E: mRNA analysis by RT-qPCR of ACTB and selected bone-related markers as indicated by the respective gene symbols (n=3).

FIGS. 3A and 3B show the expression pattern of H3K27 demethylases and cell cycle markers during osteogenic differentiation of MSCs. mRNASeq analysis of MSCs undergoing an osteogenic time-course using standard conditions (plastic cell culture vessels) (FIG. 3A) or porous-sintered titanium inserts (FIG. 3B). Graphs show expression values (reads per kilobasepair per million mapped reads; RKPM) of H3K27 demethylases (left) and cell cycle markers (right) at different days of MSC differentiation. These demethylases (KDM6A, KDM6B, and JHDM1D) remove the methyl groups that are added by the methyltransferase EZH2 on H3 lysine 27. Cell cycle markers are graphed to define the proliferative state of the differentiating MSCs.

FIGS. 4A and 4B show that miR-101a (Ezh2 targeting miR) is up-regulated during osteogenic differentiation of MC3T3 cells. Previous studies established that miR-101 modulates Ezh2 expression in different cellular systems. FIG. 4A: TargetScan predicted that several miRs control the expression of human and mouse Ezh2, including miR-101 at two binding sites in the 3′-UTR. FIG. 4B: Small RNA RT-qPCR established enhanced expression of miR-101a while miR-410 (not predicted to target Ezh2) was suppressed during osteogenic differentiation of MC3T3 cells.

FIGS. 5A-5D show that EZH2 inhibition suppresses adipogenic differentiation of MSCs. FIG. 5A: Diagram of the experimental protocol for treatment and adipogenic differentiation of MSCs with GSK126 (2 μM) shown in FIGS. 5B-5D. V=vehicle, G=2 μM GSK126. FIG. 5B: RT-qPCR analysis of representative adipogenic genes including peroxisome proliferator-activated receptor gamma (PPARG), fatty acid binding protein 4 (FABP4), and adiponectin (ADIPOQ) at day 9 of differentiation (n=3). FIG. 5C Oil Red O staining after 14 days of adipogenic differentiation. FIG. 5D: Representative microscope images (10× zoom) of cultures stained with Oil Red O are shown (right). Quantification of the Oil Red O stain (n=3).

FIGS. 6A-6C show that knock-down of H3K27 demethylase JHDM1D suppresses osteogenic differentiation of MSCs. FIG. 6A RT-qPCR analysis demonstrated successful siRNA knock-down of JHDM1D in MSCs (2 days after transfection, n=3). Alizarin red stain and (FIG. 6B) quantification (FIG. 6C) demonstrate that knock-down of JHDM1D suppresses the osteogenic lineage commitment of MSCs. Osteogenic differentiation was induced two days after transfection and alizarin red staining was performed 24 days later (n=2).

FIGS. 7A-7C depict changes in MSC gene expression after EZH2 inhibitor treatment. FIG. 7A: The top 20 functional gene annotation clusters for genes upregulated >1.4-fold in MSCs three days after EZH2 inhibitor treatment with GSK126 during osteogenic differentiation show preferential up-regulation of genes promoting a cellular anabolic state including genes linked to extracellular matrix synthesis. FIG. 7B The top 20 functional gene annotation clusters for genes downregulated >1.4-fold in MSCs three days after EZH2 inhibitor treatment with GSK126 during osteogenic differentiation show inhibition of genes that enhance mitosis and cell cycle progression. FIG. 7C: Unsupervised hierarchical clustering of more than 900 transcriptional regulators in MSCs during adipogenic, osteogenic and undifferentiated states, shows substantial changes in transcriptional profiles. EZH2 inhibitor treatment markedly changes the expression of transcriptional regulators in MSCs and represents one mechanism by which EZH2 inhibition can enhance osteogenic differentiation.

FIGS. 8A-8I show that Ezh2 inhibition promotes osteoblast maturation. FIGS. 8A and 8B: RT-qPCR analysis of mRNA expression for the indicated genes (FIG. 8A) or alizarin red staining (FIG. 8B) during maturation of MC3T3 osteoblasts with the EZH2 inhibitor GSK126 or vehicle. FIGS. 8C-8E: Western blotting reveals depletion of Ezh2 protein and H2K27Me3 relative to H3 relative to β-actin in the presence of siRNA for Ezh2 (FIG. 8C), while RT-qPCR analysis for the indicated genes (FIG. 8D) and alizarin red staining shows that Ezh2 depletion increases alizarin red staining (FIG. 8E). FIGS. 8F and 8G: Identity of all H3K27me3 marked genes (by ChIPseq) showing >1.4-fold increase in expression in MC3T3 cells treated with GSK126 versus vehicle on days 3, 6, 10 of osteogenic differentiation (FIG. 8F) and functional annotation clustering of genes using DAVID 6.757 (FIG. 8G). FIGS. 8H and 8I: Examples of genes showing decreased methylation at TSS (FIG. 8H) and enhanced expression (FIG. 8I) in MC3T3 cells treated with Ezh2 inhibitor (GSK126).

FIGS. 9A-9F show modulation of MC3T3 differentiation by siRNA and small molecule inhibition of Ezh2. FIG. 9A: Toxicity profile of GSK126 in MC3T3 cells. FIGS. 9B and 9C: Diagrams of the experimental protocols for small molecule (GSK126 and UNC1999) (FIG. 9B) and siRNA targeting of Ezh2 in MC3T3 cells (FIG. 9C). FIG. 9D: Concentration-dependent modulation of H3K27Me3 by GSK126 in MC3T3. Cells were seeded (10,000 cells/cm²) in 6-well plates, treated with different concentrations of GSK126 the next day, followed by protein harvest (3 days later) and Western blotting. FIG. 9E: Time-dependent modulation of H3K27Me3 by GSK126 in MC3T3. Cells were seeded (10,000 cells/cm²) in 6-well plates, treated with GSK126 (2 μM) the next day, protein collected at specified times, followed by western blotting. FIG. 9F: Alkaline phosphatase activity of MC3T3 cells treated with vehicle and 5 μM GSK126.

FIG. 10A-10D show that UNC1999 (an EZH2 inhibitor) promotes osteogenic differentiation of MC3T3 cells. To diminish the likelihood of drug-specific effects, another inhibitor of EZH2 (UNC1999) was utilized to verify EZH2 inhibition as a bone-anabolic strategy. The same treatment regimen was utilized with 1 μM UNC1999 as with 5 μM GSK126. FIG. 10A: RT-qPCR analysis of a house-keeping gene (Akt1) and selected bone-related markers (Sp7, Bglap, and Alpl) (n=3). FIG. 10B: Toxicity profile of UNC1999 in MC3T3 cells. Cells were treated with different inhibitor concentrations for 3 days and MTS activity was performed as described (n=3). Alkaline phosphatase (FIG. 10C) and alizarin-red (FIG. 10D) staining was carried out for MC3T3 treated with vehicle or UNC1999.

FIGS. 11A-11E show the results of mRNASeq analysis, demonstrating that EZH2 inhibition promotes osteoblast maturation. Early stages (d3, d6, and d10) of osteogenic differentiation of MC3T3 cells in the presence of vehicle or 5 μM GSK126 was assessed by mRNASeq and bioinformatics analysis. The treatment regimen is outlined in FIG. 9B. FIG. 11A: Down-regulation of Acta2 (a mesenchymal progenitor marker) is further suppressed by Ezh2 inhibition during osteogenic differentiation. FIG. 11B: Up-regulation of Cd200 (an osteogenic cluster of differentiation marker) is enhanced by Ezh2 inhibition during MC3T3 cell differentiation. FIGS. 11C-11E: Expression of osteogenic transcription factors (FIG. 11C) and extracellular matrix-related genes (FIG. 11D), including glypicans (Gpc1 and Gpc3) (FIG. 11E), is modulated by Ezh2 inhibition.

FIGS. 12A-12D provide an overview of H3K27me3 ChIPSeq data for MC3T3 cells during osteogenic differentiation. FIG. 12A: Mechanistically, enzymatic inhibition of Ezh2 using GSK126 decreases the deposition of H3K27Me3 marks near transcriptional start sites (TSSs) across the genome, based on chromatin immuno-precipitation analysis combined with next-generation DNA sequencing (ChIPSeq). FIG. 12B: Ezh2 inhibitor treatment reduces H3K27me3 in MC3T3 cells 24 hours after a single drug treatment. In total, 1990 genes show greater than 2-fold increase in FPKMs after H3K27me3 antibody pull down. In contrast, 2568 genes show a greater than 2-fold increase in FPKMs after H3K27me3 antibody pull down indicating that more genes are H3K27 tri-methylated in the non-GSK126 treated cells. The 1458 genes in the vehicle H3K27me3 pull down vs input group represent genes that are demethylated within 24 hours after a single Ezh2 inhibitor treatment. FIG. 12C: A fold change comparison of FPKM values for the input DNA (before H3K27me3 antibody pull down) from MC3T3 cells treated with GSK126 and vehicle, 24 hours after treatment as expected shows that less than 99% of the expressed genes have less than a 2-fold difference in expression. The small changes in methylation that are observed can be accounted for by differences in DNA accessibility as a result of changes in chromatin structure following GSK126 treatment. FIG. 12D: After GSK126 treatment and antibody pulldown of H3K27me3 sites, ChIPSeq data shows that there is an increase in the number of genes with greater than 2-fold change between the MC3T3 cells treated with the EZH2 inhibitor and vehicle, indicating a change in methylation status with EZH2 inhibition.

FIG. 13 is a series of graphs indicating the expression of genes showing decreased H3K27me3 with EZH2 inhibitor treatment in MC3T3 cells during osteogenic differentiation. A panel of selected genes linked to osteogenesis that show decreased H3K27me3 after Ezh2 inhibitor treatment with GSK126 (ChIPSeq, not shown) and corresponding up-regulation in gene expression (mRNASeq, shown). These genes modulate osteogenesis primarily through transcriptional regulation and cell signaling mechanisms.

FIGS. 14A-14L demonstrate the skeletal defects that arise upon conditional ablation of Ezh2 in mesenchymal precursor cells. FIGS. 14A and 14B: Images (FIG. 14A) and whole mount staining (FIG. 14B) of newborn female Ezh2+/+:Prrx1−Cre+ (WT) and Ezh2−/−:Prrx1−Cre+ (cKO) mice reveal a short stature, as well as a domed head and shortened limbs (arrows). FIGS. 14C and 14D: Radiographic images of right front limbs (FIG. 14C) or right femurs (FIG. 14D) of female WT and cKO mice at weaning; paw enlargements (selection area in FIG. 14C) show the clinodactyly phenotype. FIGS. 14E-14H: Abnormal growth plate development in cKO mice is evident in proximal tibial growth plates of newborn (FIG. 14E) or weaned (FIG. 14F) mice; the distance between the epiphysis (Epi) to the hypertrophic zone (Hyp)(arrows) and the width of the proliferative area (traced area) were examined (FIG. 14G) in male mice (newborn; WT, n=4, and cKO, n=3); the growth plate (GP) depth (dashed arrow in FIG. 14F) and hypertrophic (Hyp) depth (solid arrow in FIG. 14F) were analyzed in female WT and cKO mice (3 wk; WT or cKO: n=4) (FIG. 14H). FIGS. 14I-14L: X-ray microtomography (microCT) reconstructions of skulls (FIGS. 14I and 14J) and trabecular bone in tibia (FIG. 14K) from female WT and cKO mice (3 wk; WT or cKO: n=4).

FIGS. 15A-15F show that deletion of functional Ezh2 in the mesenchymal lineage causes skeletal defects in mice. Skeletal differences are evident between three-week old female WT and Ezh2 cKO^{Prrx1} mice. FIG. 15A: Picture demonstrating a smaller statue of cKO animals. FIG. 15B: Weight difference between cKOs (n=7) and WT (n=14) animals. FIG. 15C: X-ray images of the cephalad half of mice. FIG. 15D: Increased number of adipocytes in the tibial mid-shaft from cKO animals. X-ray (FIG. 15E) and microCT (FIG. 15F) demonstrate the presence of clavicles in cKO (albeit shorter than in WT). Arrows in the images point to phenotypic changes (short forelimbs, domed head, and clinodactyly) in Ezh2 cKO mice.

FIG. 16 is a pair of graphs demonstrating that deletion of functional Ezh2 in the mesenchymal lineage enhances the expression of osteogenic markers in mouse calvaria. To understand changes in calvarial anatomy (craniosynostosis and doming of the skull) (see FIG. 14), mRNASeq and bioinformatics analysis were performed to assess gene expression changes due to inactivation of Ezh2 in calvarial osteoblasts. Expression of osteogenic markers (e.g., Bglap, Sparc) is enhanced in the calvaria of cKO^{Prrx1} mice, which supports the phenotype of the cKO^{Prrx1} animals (craniosynostosis).

FIGS. 17A-17C illustrate differential expression of H3K27me3-regulated genes between Ezh2 cKO^{Prrx1} and wild type primary cell derived from primary calvarial digests. FIG. 17A: Approximately one third of all H3K27me3 regulated genes detected by ChIPSeq in MC3T3 cells during osteogenic differentiation show an up-regulation in Ezh2 cKO^{Prrx1} mouse calvarial cells. FIG. 17B: The top 20 functional gene annotation clusters for H3K27me3 genes upregulated in primary calvarial osteoblasts from Ezh2 cKO^{Prrx1} versus WT mice show enrichment in transcriptional regulators in the Ezh2 cKO^{Prrx1} calvarial cells. FIG. 17C HOX gene clusters, which have been shown to be regulated by Ezh2 and H3K27me3, are highly upregulated in primary osteoblasts derived from Ezh2 cKO^{Prrx1} mice. Pharmacologic Ezh2 inhibition increases expression of some of the HOX genes, but up-regulation is less pronounced than upon loss of Ezh2 function in Ezh2 cKO^{Prrx1} mice. Thus, genetic effects, cellular context and/or duration of Ezh2 inhibition may influence expression of genes controlled by H3K27me3.

FIGS. 18A and 18B show skeletal effects of deleting functional Ezh2 in the osteoblast compartment. The role of Ezh2 in the osteogenic lineage during early skeletal development was examined using the Osx-Cre driver (Ezh2 cKO^{osx}). Weight, tibia length, and structural parameters by microCT analysis of tibias from three week-old WT (n=9) and Ezh2 cKO^{Osx} (n=10) mice (FIG. 18A), or eight week-old WT (n=6) and Ezh2 cKO^{Osx} (n=8) mice (FIG. 18B). Mice initially exhibit an osteoporotic phenotype based on microCT analysis three-weeks after birth (see BV/TV, Tb.N., and ConnD), which normalizes to the wild type mice by eight weeks. Hence, early developmental effects of Ezh2 inhibition alter skeletal architecture, but do not cause osteoporosis after skeletal maturity.

FIGS. 19A-19H show bone-anabolic and osteoprotective effects of Ezh2 inhibition in skeletally mature mice. Systemic administration of EZH2 inhibitor GSK126 promotes bone formation and maintains cortical structure in vivo. FIGS. 19A-19E: Treatment of C57bl/6 mice with vehicle, 15 mg/kg or 50 mg/kg GSK126 for 5 weeks. No changes in body or spleen weight at sacrifice (FIG. 19A), suggesting no major adverse reactions. Dose-dependent bone anabolic effects of Ezh2 inhibition by microCT analysis of the femoral diaphysis (FIG. 19B). Cortical bone volume (Ct.BV), cortical area (Ct.Ar), and cortical thickness (Ct.Th.) were significantly enhanced following treatment with 50 mg/kg of GSK126 (n=3) compared to the vehicle control (n=3). Static histomorphometry analysis of the distal femoral metaphysis demonstrated increase in osteoblast surface area per bone surface area (Ob.S/BS) (FIG. 19C) and number of osteoblasts per bone perimeter (N. Ob./B.Pm) (FIG. 19D). Similarly, dynamic histomorphometric analysis performed in the same region including interlabel thickness (Ir.L.Th), mineral apposition rate (MAR) and bone formation rate (BFR) were also enhanced in the GSK126 50 mg/kg group (n=4) compared to the vehicle treated group (n=4) (FIG. 19E). FIGS. 19F-19H: The effect of GSK126 was also analyzed in a mouse ovariectomy model of hormone-deficiency osteoporosis. Six week (daily injections) were performed for each treatment group as indicated (n=4 to 5 per group) (FIG. 19F). Body and spleen weights were similar between surgical and treatment groups, while uterus weight was reduced in the ovariectomized groups (FIG. 19G). Analysis of the femoral diaphysis by microCT showed increased cortical thickness, area, tissue volume, and bone volume, and also periosteal area (FIG. 19H).

FIGS. 20A and 20B show that deletion of functional Ezh2 enhances fracture healing in mice. Intramembranous bone healing is assessed with a femoral drill-hole model in 8 week wild type (WT; FIG. 20A) and in animals in which functional Ezh2 was ablated in osteoblasts (Osx-Ezh2cKO; FIG. 20B). To induce a single-cortex defect in the femur, mice were incised above the mid-diaphysis of the femur to expose the bone, and a 0.7 mm diameter steel burr drill bit was used to create the defect. Defect healing was assessed by micro-CT 14 after surgery. These data demonstrate that deletion of Ezh2 in the osteoblasts lineage enhances bone healing in adult animals.

FIG. 21 is a series of graphs plotting synergistic activation of osteogenic genes by GSK126 and BMP2. Mesenchymal stem cells were treated with 50 ng/ml BMP2 and/or 2 μM GSK126. RNA-Seq analysis demonstrates that Ezh2 inhibition (GSK126) and BMP2 activate different set of genes, but they synergistically enhance the expression of some key osteogenic factors including DLXS (left panel), SP7 (center panel), and IBSP (right panel). These data demonstrate that EZH2 inhibition enhances the osteogenic potential of cells treated with BMP2, a key osteogenic activator.

FIG. 22A provides results of examination of changes in bone mineral density distribution (BMDD) parameters CaMean, CaPeak, CaWidth, CaLow and CaHigh, which reflect bone turnover, mineralization kinetics, and average bone matrix age by qBEI. Two-way ANOVA analysis of CaPeak and CaWidth of trabecular bone between all treatment groups. FIG. 22B is a list of RT-qPCR primers.

FIGS. 23A and 23B show that DMSO and SFN have structural similarities and analogous biological effects. DMSO and SFN each contain a polar sulfoxide functional group, and SFN carries an additional pentane group with a terminal isothiocyanate group (FIG. 23A). DMSO and SFN increase matrix mineralization in MC3T3-E1 cells at 14 days of differentiation as revealed by Alizarin Red stain (FIG. 23B).

FIGS. 24A-24F are a series of graphs indicating that SFN shows cell growth suppressive effects in cells of the osteoblastic lineage. L-SFN and DL-SFN effects on proliferation/viability in MC3T3-E1 cells (FIG. 24A) and in MLO-Y4 cells (FIG. 24B) after 24 hours and after 48 hours of SFN treatment (FIGS. 24C and 24D). The half-maximal effective concentration (EC₅₀) for L-SFN is about 48 μM and for DL-SFN is about 13 μM in MC3T3-E1 cells (FIG. 24E), and about 11 μM for L-SFN and about 6 μM for DL-SFN in MLO-Y4 cells (FIG. 24F). Cell proliferation in FIGS. 24A-24D was measured by cell count, and EC₅₀ in FIGS. 24E and 24F was measured by a MTT similar assay. n=4; bars represent mean±SD.

FIGS. 25A-25D show that SFN induces extrinsic apoptosis in cells of the osteoblastic lineage. Fas mRNA expression was determined after 3 μM L- or DL-SFN treatment in MC3T3-E1 and MLO-Y4 cells after 8 hours (FIG. 25A) and after 16 hours (FIG. 25B). Caspase 8 activity after 3 μM L-SFN and DL-SFN treatment at 24 hours was measured in both cell lines (FIG. 25C). The effect of 3 μM DL-SFN treatment in MC3T3-E1 cells and of 3 μM L-SFN and DL-SFN in MLO-Y4 cells on the activities of caspases 3/7 was assessed after 24 hours (FIG. 25D). For RT-qPCR analysis, Fas expression is referred to 18S rRNA expression (FIGS. 25A and 25B). In all graphs, Ctrl is set to 1, and treatments are referred to as fold change to Ctrl. Values are represented as the mean±SD; for FIGS. 25A and 25B n=3, for FIGS. 25C and 25D n=5, twice; *P≤0.05; **P≤0.01; ***P≤0.001.

FIGS. 26A-26C show that SNF enhances osteoblast differentiation and activity. ECM mineralization of MC3T3-E1 cells, BMSCs or neonatal calvarial explants as measured by alizarin red stain (FIG. 26A). mRNA expression of Bglap2, Colla1, Lox and Alpl after treatment with 3 μM of L- or DL-SFN of in MC3T3-E1 cells for 14 days (FIG. 26B). In BMSCs, Bglap2 mRNA expression was up regulated by DL-SFN (FIG. 26C). DL-SFN generally exhibited a more pronounced effect than L-SFN. For MC3T3-E1 and BMSCs, n=3; and for neonatal calvariae, n=9 in FIG. 26A. For RT-qPCR analysis, gene expression is referred to 18S rRNA expression, n=3 (FIGS. 26B and 26C). In all graphs, Ctrl is set to 1, and treatments are referred to as fold change to Ctrl. Values are represented as the mean±SD; *P≤0.05; **P≤0.01.

FIGS. 27A-27D show that SFN enhances expression of the osteoblastic master transcription factor Runx2. Treatment with 3 μM DL-SFN stimulated Runx2 mRNA expression in MC3T3-E1 and in BMSCs cells (FIG. 27A). In MC3T3-E1 cells, 3 μM DL-SFN significantly increased Runx2 protein expression after both 24 hours (FIG. 27B) and after 14 days (FIG. 27C) of treatment. Representative immune blots of Runx2 protein expression are presented (FIG. 27D). In FIG. 27A, for RT-qPCR analysis, Runx2 gene expression is referred to 18S rRNA expression. In FIG. 27B, Runx2 protein expression is referred to Actb expression. FIGS. 27A-27C n=3. In all graphs, Ctrl is set to 1, and treatments are referred to as fold change to Ctrl. Values are represented as the mean±SD; *P≤0.05.

FIGS. 28A and 28B show that SFN attenuates RANKL/Tnfsf11 expression in MLO-Y4 cells and mouse calvarial explants. Tnfsf11 expression in osteocyte-like MLO-Y4 cells upon 3 μM DL-SFN treatment at 3 and 8 days of treatment is presented (FIG. 28A). In mouse calvarial explants from neo-natal (5d) and from adult mice (7 we), 3 μM DL-SFN decreased Tnfsf11 expression after 12 days of treatment (FIG. 28B). Tnfsf11 gene expression is referred to 18S rRNA expression; n=3. In all graphs, Ctrl is set to 1, and treatments are referred to as fold change to Ctrl. Values are represented as the mean±SD; *P≤0.05.

FIGS. 29A and 29B indicate that SFN affects global DNA hydroxymethylation status. Global nuclear 5hmC levels (dots) in MC3T3-E1 cells after 16 hours of treatment with 3 μM DL-SFN are shown (representative images; FIG. 29A). Spectrophotometry analysis of global DNA hydroxymethylation at 16 hours after treatment with 3 μM L- and DL-SFN in MC3T3-E1 cells and in MLO-Y4 cells is presented in FIG. 29B. In FIG. 29B, n=4. Ctrl is set to 1, and treatments are referred to as fold change to Ctrl. Values are represented as the mean±SD; *P≤0.05.

FIGS. 30A-30D show that SFN transiently enhances expression of Tet1 in osteoblastic cells. Time dependent Tet1 and Tet2 expression patterns in MC3T3-E1 cells (FIG. 30A) and in MLO-Y4 cells (FIG. 30B) are shown. Expression of Tet1 and Tet2 by 3 μM DL-SFN at 8 hours and 16 hours after treatment in MC3T3-E1 cells is shown in FIG. 30C. 3 μM DL-SFN did not significantly increase mRNA expression of Tet1 or Tet2 in MLO-Y4 cells (FIG. 30D). For RT-qPCR analysis, Tet1 and Tet2 gene expressions are referred to 18S rRNA expression; n=3. In FIGS. 30C and 30D, Ctrl is set to 1, and treatments are referred to as fold change to Ctrl. Values are represented as the mean±SD; *P≤0.05.

FIG. 31A-31E show that SFN strongly inhibits viability and resorption of osteoclast. L-SFN and DL-SFN effects on RAW 264.7 cells on proliferation/viability after 24 hours of treatment are shown (FIG. 31A). For RAW 264.7 cells, the half-maximal effective concentration (EC₅₀) for L-SFN was about 10.10 μM, and for DL-SFN it was about 6.01 μM (FIG. 31B). SFN affects cell metabolic activity strongest in RAW 264.7 when compared with cells of the osteoblastic lineage (FIG. 31C). The effect of 3 μM L- or DL-SFN on dentin resorption by primary mouse osteoclast cultures is shown in FIG. 31D. A representative image of osteoclast resorption trails and pits on dentin is shown in FIG. 31E. Cell proliferation in FIG. 31A was measured by cell count. EC₅₀ in FIG. 31B was measured by a MTT similar assay. For parts A and B, n=4. For C, n=9. Bars represent mean±SD; **P≤0.01; ***P≤0.001.

FIG. 32 shows that SFN induces FAS-dependent extrinsic apoptosis pathway in pre-osteoclastic cells. mRNA expression of Fas in RAW 264.7 pre-osteoclastic cells after treatment with 3 μM of L- or DL-SFN for 16 hours is shown in FIG. 32A. Increased Caspase 8 and Caspase 3/7 activity in RAW 264.7 cells after treatment with 3 μM of either SFN preparation after 24 hours of treatment is shown in FIG. 32B. Comparison for Fas induction (FIG. 32C), activation of Caspase 8 (FIG. 32D) and Caspase 7 (FIG. 32E) activity by SFN between RAW 264.7 osteoclasts MC3T3-E1 osteoblasts and MLO-Y4 osteocytes is shown. For RT-qPCR analysis, Fas expression is referred to 18S rRNA expression. In all graphs, Ctrl is set to 1, and treatments are referred to as fold change to Ctrl. Values are represented as the mean±SD. For A, n=3. For B, n=5, twice; *P≤0.05; **P≤0.01; ***P≤0.001.

FIG. 33 indicates that SFN induces global DNA hydroxymethylation and induces Tet1 dependent cell death in RAW 264.7 pre-osteoclasts. Global nuclear 5hmC levels (dots) in RAW 264.7 cells after 16 hours of treatment with 3 μM DL-SFN are shown (FIG. 33A). FIG. 33 B provides a quantitative analysis in global 5hmC by treatment with 3 μM L-SFN and DL-SFN in RAW 264.7 cells after 16 hours. A comparison of changes in global 5hmC levels between RAW 264.7, MC3T3-E1 and MLO-Y4 cells upon 3 μM DL-SFN treatment after 16 hours is shown in FIG. 33C. The effect of 3 μM L- or DL-SFN on Tet1 and Tet2 mRNA expression after 8 and after 16 hours of treatment in RAW 264.7 cells is shown in FIG. 33D. FIG. 33E shows validation of mRNA expression knock down by specific siRNAs against Tet1 and Tet2 in RAW 264.7 cells. The effect of Tet1 and Tet2 knock down on the cytostatic effect induced by DL-SFN in RAW 264.7 cells is shown in FIG. 33F. In FIG. 33A, representative images are shown. In FIG. 33B, n=4. For RT-qPCR analysis, Tet1 and Tet2 gene expressions are referred to 18S rRNA expression, n=3. F is measured by cell count, n=4. In all graphs, Ctrl is set to 1, and treatments are referred to as fold change to Ctrl. Values are represented as the mean±SD; *P≤0.05; **P≤0.01; ***P≤0.001.

FIG. 34 is a schematic overview of the effects of SFN/DMSO on bone cells. DMSO and SFN induced active DNA demethylation via up-regulation of the Tet genes in vitro. This leads to apoptosis of pre-osteoclasts and to a lesser extent of pre-osteoblasts. Active DNA demethylation also enhanced osteoblast differentiation. SFN further decreased the expression of RANKL/Tnfsf11 by a yet undefined mechanism. mCpG: methylated cytosines; hmCpG: hydroxymethylated cytosines; CpG: unmethylated CpGs; TSS: transcriptional start site.

FIGS. 35A-35D depict the anabolic effect of SFN on bone homeostasis in C57BL/6 mice. The effects of treatment of sham-operated and ovariectomized (OVX) young adult mice (8 week old) with 7.5 mM DL-SFN for 5 weeks on trabecular bone volume over total tissue volume (BVTV) (FIG. 35A), trabecular number (Tb.N) (FIG. 35B), trabecular separation (Tb.Sp) (FIG. 35C) and trabecular thickness (Tb.Th) (FIG. 35D) are shown. n=9 for the sham group, n=7 for the Ctrl OVX group, and n=8 for the DL-SFN group. Values are represented as the mean±SD; *P≤0.05; ***P≤0.001.

FIGS. 36A-36F present evidence correlating matrix mineralization parameters (qBEI) with structural parameters (μCT). The parameters CaPeak (the most frequent calcium content) and CaWidth (the width of the calcium content distribution) correlated significantly with the corresponding μCT parameters BV/TV (FIGS. 36A and 36B), Tb.N (FIGS. 36C and 36D), and Tb.Sp (FIGS. 36E and 36F).

### DETAILED DESCRIPTION

This document provides methods and materials for promoting bone formation for the treatment of conditions such as osteoporosis, bone defects, bone injury, joint fusion, and implant ingrowth. For example, this document provides methods and materials for using sulforaphane and/or EZH2 polypeptide inhibitors (e.g., GSK126 or UNC1999) to promote bone formation, to improve cortical bone structure, to reverse osteoporosis, to slow the progression of osteoporosis, and/or to prevent the onset of osteoporosis.

Any type of mammal having osteoporosis, at risk for developing osteoporosis, or having another condition in which bone growth would be beneficial (e.g., a bone defect or injury, a bone implant such as a hip or knee replacement, or joint/spinal fusion) can be treated as described herein. For example, humans and other primates such as monkeys having osteoporosis can be treated with sulforaphane, one or more EZH2 polypeptide inhibitors, or a combination of sulforaphane and one or more EZH2 polypeptide inhibitors. In some cases, dogs, cats, horses, cows, pigs, sheep, rabbits, mice, and rats can be treated with sulforaphane and/or one or more EZH2 polypeptide inhibitors as described herein.

Any appropriate method can be used to identify a mammal having osteoporosis or as being at risk for developing osteoporosis. For example, a bone mineral density (BMD) test using dual-energy x-ray absorptiometry (DXA test), micro-computed tomography (micro-CT) and other forms of CT-based absorptiometry (e.g., quantitative computed tomography, QCT; peripheral QCT, pQCT), bone turnover markers (BTMs) in serum (e.g., CTX, P1NP, BGLAP), or quantitative ultrasound densitometry (QUS) techniques can be used to identify a human or other mammal having osteoporosis.

Once identified as having osteoporosis, being at risk for developing osteoporosis, or having another condition in which new bone formation would be beneficial, the mammal can be administered or instructed to self-administer sulforaphane, one or more EZH2 polypeptide inhibitors, or a combination of sulforaphane and one or more EZH2 polypeptide inhibitors. Examples of EZH2 polypeptide inhibitors include, without limitation, (a) GSK126 (N-[(1,2-dihydro-4,6-dimethyl-2-oxo-3-pyridinyl)methyl]-3-methyl-1-[(1S)-1-methylpropyl]-6-[6-(1-piperazinyl)-3-pyridinyl]-1H-indole-4-carboxamide), (b) UNC1999 (N-[(6-methyl-2-oxo-4-propyl-1,2-dihydropyridin-3-yl)methyl]-1-(propan-2-yl)-6-{6-[4-(propan-2-yl)piperazin-1-yl]pyridin-3-yl}-1H-indazole-4-carboxamide), (c) EPZ005687 (1-Cyclopentyl-N-((4,6-dimethyl-2-oxo-1,2-dihydropyridin-3-yl)methyl)-6-(4-(morpholinomethyl)phenyl)-1H-indazole-4-carboxamide), (d) GSK343 (N-[(6-methyl-2-oxo-4-propyl-1,2-dihydropyridin-3-yl)methyl]-6-[2-(4-methylpiperazin-1-yl)pyridin-4-yl]-1-(propan-2-yl)-1H-indazole-4-carboxamide), (e) EPZ-6438 (CAS No. 1403254-99-8), and (f) EI1 (CAS No. 1418308-27-6). In some cases, a sulforaphane alternative such as SULFORADEX® (Evgen Pharma; a product containing synthetic sulforaphane and α-cyclodextrin), methylsulfonylmethane (MSM) (CAS No. 67-71-0), or a thiocyanate containing compound can be used in addition to sulforaphane or in place of sulforaphane. Examples of thiocyanate containing compounds include, without limitation, Erucin (CAS No. 4430-36-8), Lipoic acid (LA)/α-lipoic acid/alpha lipoic acid (ALA)/thioctic acid (CAS No. 1200-22-2 & 1077-28-7), allyl isothiocyanate (AITC)/3-Isothiocyanato-1-propene (CAS No. 57-06-7), phenyl isothiocyanate (PITC) (CAS No. 103-72-0), pentyl isothiocyanate (CAS 629-12-9), and allicin/2-propene-1-sulfinothioic acid S-2-propenyl ester (CAS 539-86-6). For example, a human having osteoporosis can be treated with sulforaphane or sulforaphane alternatives (e.g., any member of the family of thiocyanate containing compounds listed above) in combination with one or more EZH2 polypeptide inhibitors.

In some cases, sulforaphane (and/or one or more sulforaphane alternatives) and one or more EZH2 polypeptide inhibitors (e.g., one, two, three, four, five, or more EZH2 polypeptide inhibitors) can be administered to a mammal to treat osteoporosis (e.g., to reverse osteoporosis). For example, sulforaphane and two or more EZH2 polypeptide inhibitors can be administered to a mammal (e.g., a human with osteoporosis) to treat osteoporosis (e.g., to reverse osteoporosis). In some cases, sulforaphane (and/or one or more sulforaphane alternatives) and/or one or more EZH2 polypeptide inhibitors can be formulated into a pharmaceutically acceptable composition for administration to a mammal having osteoporosis or as being at risk for developing osteoporosis. For example, a therapeutically effective amount of sulforaphane and GSK126 can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing sulforaphane (and/or one or more sulforaphane alternatives) and/or one or more EZH2 polypeptide inhibitors can be designed for oral, parenteral (including subcutaneous, intramuscular, intravenous, and intradermal), or local administration. When being administered orally, a pharmaceutical composition containing sulforaphane (and/or one or more sulforaphane alternatives) and/or one or more EZH2 polypeptide inhibitors can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

Such injection solutions can be in the form, for example, of a sterile injectable aqueous or oleaginous suspension. This suspension may be formulated using, for example, suitable dispersing or wetting agents (such as, for example, Tween 80) and suspending agents. The sterile injectable preparation can be a sterile injectable solution or suspension in a non-toxic parenterally-acceptable diluent or solvent, for example, as a solution in 1, 3-butanediol. Examples of acceptable vehicles and solvents that can be used include, without limitation, mannitol, water, Ringer's solution, and isotonic sodium chloride solution. In addition, sterile, fixed oils can be used as a solvent or suspending medium. In some cases, a bland fixed oil can be used such as synthetic mono- or di-glycerides. Fatty acids, such as oleic acid and its glyceride derivatives can be used in the preparation of injectables, as can natural pharmaceutically-acceptable oils, such as olive oil or castor oil, including those in their polyoxyethylated versions. In some cases, these oil solutions or suspensions can contain a long-chain alcohol diluent or dispersant.

In some cases, a pharmaceutically acceptable composition including sulforaphane (and/or one or more sulforaphane alternatives) and/or one or more EZH2 polypeptide inhibitors can be administered locally or systemically. For example, a composition containing sulforaphane (and/or one or more sulforaphane alternatives) and/or one or more EZH2 polypeptide inhibitors can be administered systemically by an oral administration or by injection to a mammal (e.g., a human). A pharmaceutically acceptable composition also may be delivered locally, such as to a surgical site, using a carrier such as a collagen sponge or polymer-based sponge or scaffold, or another carrier device.

Effective doses can vary depending on the severity of the condition (e.g., osteoporosis or bone defect/injury), the route of administration, the age and general health condition of the subject, excipient usage, the possibility of co-usage with other therapeutic treatments such as use of other agents, and the judgment of the treating physician.

An effective amount of a composition containing sulforaphane (and/or one or more sulforaphane alternatives) and/or one or more EZH2 polypeptide inhibitors can be any amount that reduces the severity of a symptom of a condition being treated (e.g., osteoporosis or bone defect/injury) without producing significant toxicity to the mammal. For example, an effective amount of sulforaphane can be from about 0.01 mg/kg to about 50 mg/kg (e.g., from about 0.1 mg/kg to about 50 mg/kg, from about 1 mg/kg to about 50 mg/kg, from about 5 mg/kg to about 50 mg/kg, from about 10 mg/kg to about 50 mg/kg, from about 0.01 mg/kg to about 25 mg/kg, from about 0.01 mg/kg to about 10 mg/kg, from about 0.01 mg/kg to about 5 mg/kg, from about 0.01 mg/kg to about 1 mg/kg, from about 0.1 mg/kg to about 10 mg/kg, or from about 1 mg/kg to about 5 mg/kg). In some cases, between about 0.5 g and 6 g either daily or twice-weekly of sulforaphane can be administered to an average sized human (e.g., about 65-75 kg human) for about four to about eight weeks (e.g., about five to six weeks).

An effective amount of an EZH2 polypeptide inhibitor such as GSK126 can be from about 0.01 mg/kg to about 50 mg/kg (e.g., from about 0.1 mg/kg to about 50 mg/kg, from about 1 mg/kg to about 50 mg/kg, from about 5 mg/kg to about 50 mg/kg, from about 10 mg/kg to about 50 mg/kg, from about 0.01 mg/kg to about 25 mg/kg, from about 0.01 mg/kg to about 10 mg/kg, from about 0.01 mg/kg to about 5 mg/kg, from about 0.01 mg/kg to about 1 mg/kg, from about 0.1 mg/kg to about 10 mg/kg, or from about 1 mg/kg to about 5 mg/kg). In some cases, between about 3 g twice-weekly of an EZH2 polypeptide inhibitor such as GSK126 can be administered to an average sized human (e.g., about 65-75 kg human) daily for about four to about eight weeks (e.g., about five to six weeks). If a particular mammal fails to respond to a particular amount, then the amount of sulforaphane and/or the amount of an EZH2 polypeptide inhibitor can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., osteoporosis or bone defect/injury) may require an increase or decrease in the actual effective amount administered.

The frequency of administration can be any frequency that reduces the severity of a symptom of a condition to be treated (e.g., osteoporosis or bone defect/injury) without producing significant toxicity to the mammal. For example, the frequency of administration can be from about once a week to about three times a day, from about twice a month to about six times a day, or from about twice a week to about once a day. The frequency of administration can remain constant or can be variable during the duration of treatment. A course of treatment with a composition containing sulforaphane (and/or one or more sulforaphane alternatives) and/or one or more EZH2 polypeptide inhibitors can include rest periods. For example, a composition containing sulforaphane (and/or one or more sulforaphane alternatives) and/or one or more EZH2 polypeptide inhibitors can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., osteoporosis or bone defect/injury) may require an increase or decrease in administration frequency.

An effective duration for administering a composition containing sulforaphane (and/or one or more sulforaphane alternatives) and/or one or more EZH2 polypeptide inhibitors can be any duration that reduces the severity of a symptom of the condition to be treated (e.g., osteoporosis or bone defect/injury) without producing significant toxicity to the mammal. For example, the effective duration can vary from several days to several weeks, months, or years. In some cases, the effective duration for the treatment of osteoporosis can range in duration from about one month to about 10 years. Multiple factors can influence the actual effective duration used for a particular treatment. For example, an effective duration can vary with the frequency of administration, effective amount, use of multiple treatment agents, route of administration, and severity of the condition being treated.

In certain instances, a course of treatment and the severity of one or more symptoms related to the condition being treated (e.g., osteoporosis or bone defect/injury) can be monitored. Any appropriate method can be used to determine whether or not the severity of a symptom is reduced. For example, the severity of a symptom of osteoporosis (e.g., bone mass density) can be assessed using DEXA, micro-CT, quantitative ultrasound densitometry, or blood serum markers at different time points. In the case of fracture healing, bony repair can be evaluated using technologies such as x-ray, CT, or MRI imaging modalities.

In some cases, sulforaphane (and/or one or more sulforaphane alternatives) and/or one or more EZH2 polypeptide inhibitors (e.g., one, two, three, four, five, or more EZH2 polypeptide inhibitors) can be used in combination with an anti-osteoporosis agent. For example, sulforaphane and one or more EZH2 polypeptide inhibitors can be administered together or separately with one or more anti-osteoporosis agents within days (e.g., one, two, three, four, or more days) or months (e.g., one, two, three, or four months) of each other to a mammal to treat osteoporosis (e.g., to reverse osteoporosis). Further, one or more EZH2 polypeptide inhibitors (e.g., one, two, three, four, five, or more EZH2 polypeptide inhibitors) can be used in combination with one or more bone anabolic compounds, such as BMP2, to promote bone repair, fracture healing, implant ingrowth, and/or joint/spine fusion.

Examples of anti-osteoporosis and bone anabolic agents that can be used in conjunction with EZH2 polypeptide inhibitors include, without limitation, bisphosphonates (e.g., alendronate, alendronate sodium, alendronate sodium/cholecalciferol, clodronic acid, etidronate, etidronate disodium, ibandronate, ibandronate sodium, olpadronate, pamidronate, pamidronate disodium, risedronate, risedronate sodium, tiludronate, tiludronate disodium, and zoledronic acid), parathyroid hormone (PTH), teriparatide, bone morphogenic protein (e.g., BMP2), hormone replacement therapies (e.g., estradiol, estropipate, and calcitonin), calcitriol, denosumab, cholecalciferol, ergocalciferol, hydrochlorothiazide, iloprost, strontium ranelate, tamoxifen citrate, calcium carbonate, calcium citrate, vitamin D, and raloxifene.

The invention will be further described in the following examples, which do not limit the scope of the invention described in the claims.

### EXAMPLES

### Example 1—Using EZH2 Polypeptide Inhibitors to Treat Osteoporosis

### Cell Culture

Mesenchymal stromal cells were derived from lipo-aspirates obtained from consenting healthy donors as described elsewhere (Crespo-Diaz et al., Cell Transplant., 20:797-811 (2011) and Mader et al., J. Transl. Med., 11:20 (2013)). Fat tissue was enzymatically digested using 0.075% Type I collagenase (Worthington Biochemicals) for 1.5 hours at 37° C. Adipocytes were separated from the stromal vascular fraction by low speed centrifugation (400 g for 5 minutes). The adipose supernatant was removed, and the cell pellet was rinsed with PBS and passed through 70 and 40 μm cell strainers (BD Biosciences). The resulting adipose-derived mesenchymal cell (AMC) fraction was maintained in Advanced MEM Medium containing 5% PLTMAX® (a clinical grade commercial platelet lysate product obtained from Mill Creek Life Sciences), 2 mM GLUTAMAX™ (Invitrogen), 2 U/mL heparin (hospital pharmacy), 100 U/mL penicillin, and 100 μg/mL streptomycin (CELLGRO®) as described elsewhere (Crespo-Diaz et al., Cell Transplant., 20:797-811 (2011)). MC3T3 sc4 murine calvarial osteoblasts (Wang et al., J. Bone Miner. Res., 14:893-903 (1999)) were purchased from ATCC and maintained in αMEM without ascorbic acid (Invitrogen) containing 10% FBS (Gibco), 100 U/mL penicillin, and 100 μg/mL streptomycin.

### 3-(4,5-dimethylthiazol-2-yl)-5-(3-carboxymethoxyphenyl)-2-(4-sulfophenyl)-2H-tetrazolium, Inner Salt (MTS) Activity Assay

AMCs or MC3T3 cells were plated in 96-well plates in maintenance medium (5,000 cells/well). The following day, vehicle (DMSO) or EZH2 inhibitors (GSK126 and UNC1999) in fresh maintenance medium were added to the cells. Three days later, MTS activity assays were performed according to manufacturer's protocol (Promega). Absorbance was measured at 490 nm using a SPECTRAMAX® Plus (Molecular Devices) spectrophotometer.

RNA Isolation from Mouse Calvaria

Calvaria (parietal and frontal bones) from 3 day-old WT and cKOPrrx1 pups were harvested and frozen as described elsewhere (McGee-Lawrence et al., Bone, 66:277-286 (2014)). Tissues were homogenized in QIAZOL® reagent (Qiagen) using a high-speed disperser (ULTRA-TURRAX® T25, IKA), and RNA was isolated from the tissue using the miRNeasy kit.

mRNA Quantitative Real-Time Reverse Transcriptase PCR (RT-qPCR)

RNA was isolated using the miRNeasy kit (Qiagen). Isolated RNA was reverse transcribed into cDNA using the SUPERSCRIPT® III First-Strand Synthesis System (Invitrogen). Gene expression was quantified using real-time PCR whereby each reaction was performed with 10 ng cDNA per 10 μL, the QUANTITECT® SYBR® Green PCR Kit (Qiagen), and the CFX384 Real-Time System machine (BioRad). Transcript levels were quantified using the 2^{ΔΔCt} method and normalized to the housekeeping gene GAPDH/Gapdh (set at 100).

### Western Blotting

AMCs (4,000 cells/cm²) or MC3T3 (10,000 cells/cm²) were plated in 6-well plates in maintenance medium. Cells were treated with vehicle or EZH2 inhibitors (GSK126 and UNC1999) as described herein. Cells were lysed in radio-immunoprecipitation buffer (150 mM NaCl, 50 mM Tris pH 7.4, 1% sodium deoxycholate, 0.1% sodium dodecyl sulfate, and 1% Triton X-100) supplemented with protease inhibitor cocktail (Sigma) and phenylmethylsulphonyl fluoride (Sigma). Lysates were cleared by centrifugation. Protein concentrations were determined by the DC Protein Assay (Bio-Rad). Proteins were resolved by SDS-PAGE and transferred to polyvinylidene difluoride membranes. After blocking in 5% non-fat dry milk for 45 minutes at room temperature, primary antibodies were added overnight at 4° C., followed by secondary antibodies for 1 hour at room temperature. Proteins were visualized using an ECL Prime detection kit. The following primary antibodies were used: Tubulin (1:10,000; E7; Univ. of Iowa), Actin (1:10,000; sc-1616; Santa Cruz), H3 (1:10,000; 05-928; Millipore), H3K27Me3 (1:5,000; 17-622; Millipore), and EZH2 (1:10,000; 5246; Cell Signaling).

### EZH2 Inhibition and Osteogenic Differentiation

AMCs cells were seeded in 6-well plates in maintenance medium (4,000 cells/cm²). The following day, maintenance medium was replaced with osteogenic medium (maintenance medium with human osteogenic supplement (R&D Systems)) containing vehicle or EZH2 inhibitor. Three days later, GSK126 and vehicle were removed and fresh osteogenic medium added.

MC3T3 cells were seeded in 6-well plates in maintenance medium (10,000 cells/cm²). The following day, maintenance medium was replaced with osteogenic medium (αMEM supplemented with 50 μg/mL ascorbic acid (Sigma) and 4 mM beta glycerol phosphate (Sigma)) containing vehicle or EZH2 inhibitor. Three days later, vehicle or EZH2 inhibitors were added again with osteogenic medium. On day six, EZH2 inhibitor and vehicle were removed, and fresh osteogenic medium added.

Media were changed every three days. RNA was isolated at indicated times. On day 6, cells were fixed in 10% neutral buffered formalin and stained with 5-bromo-4-chloro-3-indolyl-phosphate/nitro blue tetrazolium to monitor the enzymatic activity of alkaline phosphatase (Promega). On day 14 (AMCs) and days 21-25 (MC3T3), cells were fixed in 10% neutral buffered formalin and stained with 2% Alizarin Red to visualize calcium deposition. Absorption of alizarin red stain was normalized to total DNA as measured by Hoechst staining of the nuclei.

### Ezh2 Knock-Down and Osteogenic Differentiation

AMCs cells were seeded in 6- or 12-well plates in maintenance medium (4,000 cells/cm²). The following day, siRNA transfections with control (D-001810-10-20, GE Lifesciences), human EZH2 (L-004218-00, GE Lifesciences), and human JHDM1D (SASI_Hs02 00358223, Sigma) were performed using LIPOFECTAMINE® RNAiMAX (Invitrogen) as instructed by manufacturer in platelet lysate free conditions. Five hours later, AMCs osteogenic medium was added, and the cells were cultured as described.

MC3T3 cells were seeded in 6- or 12-well plates in maintenance medium (10,000 cells/cm²). The following day, siRNA transfections with control and mouse Ezh2 (L-040882-00, GE Lifesciences) were performed using RNAiMAX as instructed by manufacturer. The following day, MC3T3 osteogenic medium was added, and the cells were cultured as described.

### Ezh2 Inhibition and Adipogenic Differentiation

AMCs were seeded in 6-well plates in maintenance medium (4,000 cells/cm²). The following day, maintenance medium was replaced with adipogenic medium (maintenance medium with human adipogenic supplement (R&D Systems)) containing vehicle or EZH2 inhibitor. Three days later, vehicle and EZH2 inhibitor were removed, and fresh adipogenic medium was added. Media were changed every three days. RNA was isolated at indicated times. On day 14, cells were fixed in 10% neutral buffered formalin and stained with Oil Red O (Sigma) which binds to lipids and neutral triglycerides. Oil Red O staining was further visualized using 10× magnification with an inverted microscope. Finally, Oil Red O stain was dissolved in isopropyl alcohol, and optical density was measured at 490 nM using a spectrophotometer.

miRNA Isolation and Real-Time Reverse Transcriptase PCR (RT-qPCR)

MC3T3 cells were seeded in 6-well plates in maintenance medium (10,000 cells/cm²). The following day, maintenance medium was replaced with osteogenic medium, and RNA was harvested at day 0, 4, 7 and 14 of differentiation. RNA was isolated miRNeasy kit according to the manufacturer, and 1 μg of RNA was used as template for poly-A labelling. Reverse transcription reactions were performed using the QUANTIMIR™ RT kit small RNA quantitation system (System Biosciences). The real-time qPCR reaction was performed using IQ™ SYBR® Green Supermix (Bio-Rad), a universal reverse primer, and a miRNA specific forward primer. The products were detected using the 384-CFX real time PCR machine (BioRad). Expression of miRNAs were normalized to U6 (set at 100), using the 2^{ΔΔCt} method.

### Osteogenic Time-Course Differentiation of AMCs Grown on Plastic and Titanium Disks

For AMCs differentiation of plastic, AMCs cells were seeded in 6-well plates in maintenance medium (4,000 cells/cm²). Three days later (˜80% confluence), RNA was collected as d-1 sample. The next day (confluent culture), RNA was isolated as d0. Other plates were differentiated in osteogenic medium, and RNA was collected on various days of differentiation (7, 14, and 21). The medium was replaced every 2-3 days.

Highly porous structured titanium discs (Ti6A14V; 3 mm height; 25 mm diameter) were obtained from Stryker Mako. 6-well plates were coated with Poly 2-hydroxyethyl methacrylate (Sigma). To accomplish adsorption, about 2.5×10⁵ cells, suspended in 500 μL, were applied to the surface of each ps-Ti disc placed in the well of a 6-well plate. An additional 500 μL of medium was added to the bottom of each well to prevent desiccation of the cells during adsorption. After 2 hours of adsorption in an incubator, enough media was added to each well to submerge the entire disc (3 mL; Day −1). Following adsorption, the cells were incubated for 24 hours, at which time osteogenic medium was added to each cell type. Osteogenic media changes were performed every 2-3 days throughout the 21-day duration of the study. RNA was collected on various days of differentiation (0, 3, 7, 14, and 21).

### Deletion of Function Ezh2 in Mesenchymal and Osteoblasts Lineages in Mice

Mice containing a conditional Ezh2fl/fl allele (Su et al., Nat. Immunol., 4:124-131 (2003)) and harboring two loxP sites flanking the SET domain were obtained from a Mutant Mouse Regional Resource Center (B6; 129P2-Ezh2tm1Tara/Mmnc University of North Carolina, Chapel Hill). Ezh2 function was conditionally ablated in uncommitted mesenchymal cells or osteoprogenitors by mating with mice expressing Cre recombinase from the Prrx1 enhancer (Logan et al., Genesis, 33:77-80 (2002)) or the bone-specific Osx promoter (Rodda and McMahon, Development, 133:3231-3244 (2006)), respectively. These crosses generated wildtype (WT, Ezh2 wt/wt: Prrx1⁻Cre⁺ or Osx⁻Cre⁺) and conditional knock-out (cKO, Ezh2fl/fl: Prrx1⁻Cre⁺ or Osx⁻Cre⁺) animals. The mice were on the C57Bl/6 genetic background.

The following primers were used for genotyping Ezh2 and Cre:

Ezh2 Forward:
(SEQ ID NO: 1)
TGTCATGTCTGGGTCTAATGCTAC
Ezh2 Reverse:
(SEQ ID NO: 2)
GGAACCTCGCTATGTGTAACCA
Cre Forward:
(SEQ ID NO: 3)
TCCAATTTACTGACCGTACACCAA
Cre Reverse:
(SEQ ID NO: 4)
CCTGATCCTGGCAATTTCGGCTA.

Animals were housed in an accredited facility under a 12-hour light/dark cycle and provided water and food (PICOLAB® Rodent Diet 20, LabDiet) ad libitum.

### Whole Mount Staining

Skeletons from one day-old female mouse pups were dissected and fixed overnight in ethanol. Cartilage was stained with a 0.2% Alcian blue dye (dissolved in 80% ethanol and 20% glacial acetic acid) for 24 hours. Skeletons were washed twice with 95% ethanol and then placed in 2% KOH until the remaining soft tissues were dissolved. Bones were stained with 75 μg/mL Alizarin Red (Sigma) in 1% KOH overnight and de-stained (20% glycerol, 1% KOH) for 2 weeks, with daily solution changes. Skeletons were transferred to a 20% glycerol, 20% ethanol solution overnight and then stored in a 50% glycerol, 50% ethanol solution. Images of tissues were obtained using a Wild M420 Macroscope (Wild Heerbrugg) and PROGRES® C3 camera (Jenoptik).

### Histological Assessments

Tibias from one day-old male or three week-old female Ezh2 cKO or WT mice (Prrx1-Ezh2) were fixed in 10% neutral buffered formalin, decalcified in 15% EDTA for 7 days, paraffin embedded, sectioned, and stained with Alcian blue (1% Alcian blue, 3% acetic acid) and Eosin. The distance from the epiphysis to the hypertrophic zone in one day-old mice was assessed by taking the average of four measurements from each mouse using image J software. The proliferative area of these mice also was assessed. Total, proliferative and hypertrophic growth plate depths from three week-old animals were determined by taking the average of 15 measurements across the imaged growth plate from each mouse using Image J software. Images from the mid-shaft marrow cavity also were collected.

### Micro-Computed Analysis of Ezh2 Deletion Mice

Bone architecture of three or eight week-old mice was evaluated in the proximal tibia and skull using ex vivo micro-computed tomography (microCT). Bones were scanned in 70% ethanol on a μCT35 scanner (Scanco Medical AG, Basserdorf) at 7 μm voxel size (tibias) or 20 μm voxel size (skulls) using an energy setting of 70 kVp and an integration time of 300 ms. For the proximal tibia scans, a region of interest spanning from 17% to 20% of total bone length (relative to the proximal epiphysis) was analyzed in each mouse (threshold=220). Trabecular bone volume fraction (Tb. BV/TV, %), trabecular number (Tb.N, mm⁻¹), trabecular thickness (Tb.Th, mm), trabecular separation (Tb.Sp, mm), connectivity density (ConnD, 1/mm³), and structure model index (SMI) were computed using the manufacturer's software (Bouxsein et al., J. Bone Miner. Res., 25:1468-1486 (2010)). Skulls were reconstructed using a threshold value of 125 for gross observation of calvarial and suture morphology.

### High Throughput RNA Sequencing and Bioinformatic Analysis

mRNASeq was performed on the following samples: 1) MSCs grown on plastic or titanium disks in osteogenic cocktail (one sample from each time-point, samples created by pooling biological triplicates), 2) RNA from d3 AMCs treated with vehicle or 2 μM GSK126 in osteogenic cocktail (three samples from each treatment group), 3) RNA from d3, d6, and d10 MC3T3 treated with vehicle or 5 μM GSK126 (one sample from each treatment group at each time-point, samples created by pooling biological triplicates), 4) RNA from three day old female WT or cKO (Prrx1-Ezh2) (one sample from each genotype, pooling of RNA from three (WT) and two (cKO)), and one sample of MSCs in adipogenic cocktail for six days. High throughput mRNA sequencing and bioinformatic analyses (mRNASeq) were performed as described elsewhere (Dudakovic et al., J. Cell Biochem., 115:1816-1828 (2014)).

### ChIP-Seq and Bioinformatics Analysis

MC3T3 (10,000 cells/cm²) were plated in 10 cm plates in maintenance medium. Two days later, 5 μM GSK126 or vehicle were added to the cells in osteogenic medium. Twenty four hours later, cells were harvested by trypsin, and a chromatin immunoprecipitation assay (ChIP) was performed as described elsewhere (Dudakovic et al., J. Biol. Chem., 288:28783-28791 (2013)) using H3K27Me3 (17-622, Lot 2213948, Millipore) and IgG (PP64B, Lot 2056666A, Millipore) antibodies.

Sequencing libraries were prepared and massively parallel high throughput sequencing was performed on Illumina HiSeq2000 system. The alignment, quality assessment, peak calling, and visualization were performed with the HiChIP analysis pipeline (Yan et al., BMC Bioinformatics, 15:280 (2014)). Briefly, the 50 base-pair reads were aligned to the mm10 reference genome using Burrows-Wheeler Aligner. Duplicates were marked with Picard (http://broadinstitute.github.io/picard), and read-pairs without a unique alignment were filtered out using SAMTools (Li et al., Bioinformatics, 25:2078-2079 (2009)). Duplicates were filtered out using a custom script, and pairs with one or both ends mapped uniquely were retained. Enriched regions were identified using SICER (Zang et al., Bioinformatics, 25:1952-1958 (2009)).

### In Vivo Ezh2 Inhibition Studies

Female C57BL/6 mice (Harlan Laboratories) were maintained on a 12-hour light/12-hour dark cycle and were permitted ad libitum access to food and water. For efficacy studies, 6-week old mice received daily intraperitoneal (IP) injections of GSK126 at 15 mg/kg, 50 mg/kg, or vehicle (DMSO) in 20% Captisol adjusted to pH 4-4.5 with IN acetic acid (McCabe et al., Nature, 492:108-112 (2012)) for 5 weeks.

The dosage, delivery schedule, and administration route were similar to those described elsewhere (McCabe et al., Nature, 492:108-112 (2012)). Animals were weighed daily. To label mineralizing bone surfaces, mice received subcutaneous injections of calcein (10 mg/kg) 5 days and 24 hours before euthanasia.

The effects of GSK126 administration on the skeleton was evaluated in an estrogen-deficient ovariectomy (OVX) model. At about 12 weeks of age, female C57BL/6 mice underwent either sham or OVX surgeries. The following day, animals received daily IP injections of GSK126 or vehicle (DMSO) at 50 mg/kg body weight for 6 weeks as described herein. To label mineralizing bone surfaces and study bone formation rates, mice received subcutaneous injections of tetracycline (25 mg/kg) 14 days prior sacrifice and calcein (10 mg/kg) at 5 days and 24 hours before euthanasia.

### Bone Histomorphometry

The right distal femur was processed for static and dynamic histomorphometry as described elsewhere (McGee-Lawrence et al., Bone, 48:1117-1126 (2011)). Thin (5 μm) sections were stained with Von Kossa/McNeal's tetrachrome to highlight osteoblast surfaces and Goldner's trichrome to examine mineralizing surface and bone surfaces. Osteoclasts were detected using TRAP staining. Unstained sections were used for assessment of dynamic histomorphometry and bone area. Slides were digitized under 40× magnification using Mikroscan D2 digital whole slide scanner and Q-Skan software (Mikroscan Technologies). Beginning 450 μm proximal to the growth plate, mineralizing surface (MS/BS, %/day), mineral apposition rate (MAR, μm/day), bone formation rate (BFR/BV, %/day), osteoblast surface/bone surface (Ob.S/BS, %), osteoblast number/bone perimeter (N.Ob/B.Pm, #/mm), osteoclast surface/bone surface (Oc.S/BS, %), and osteoclast number/bone perimeter (N.Oc/B.Pm, #/mm) were quantified using Image J software as described elsewhere (Schneider et al., Nat. Methods, 9:671-675 (2012) and Egan et al., Histopathology, 61:1168-1173 (2012)).

### Micro-Computed Analysis of In Vivo Ezh2 Inhibition Studies

The quantitative analysis of the femoral metaphysis was performed using a vivaCT 40 scanner (SCANCO Medical AG) with the following parameters: E=55 kVp, 1=145 μA, integration time=300 ms. A 10.5 μm voxel size using a threshold of 220 was applied to all scans at high resolution. Using two-dimensional data from scanned slices, 3D analysis was generated and used to calculate morphometric parameters defining trabecular bone mass and micro-architecture, including Bone Volume/Tissue volume (BV/TV), trabecular number (Tb.N.), trabecular thickness (Tb.Th.), trabecular spacing (Tb.Sp.), and the structure model index (SMI), an indicator of plate-like versus rod-like trabecular architecture.

### Statistics

Statistical analysis was performed with unpaired Student's t-test (Excel, Microsoft). Significance is noted in the figures, when applicable (*: p<0.05, **: p<0.01, and ***: p<0.001).

### Results

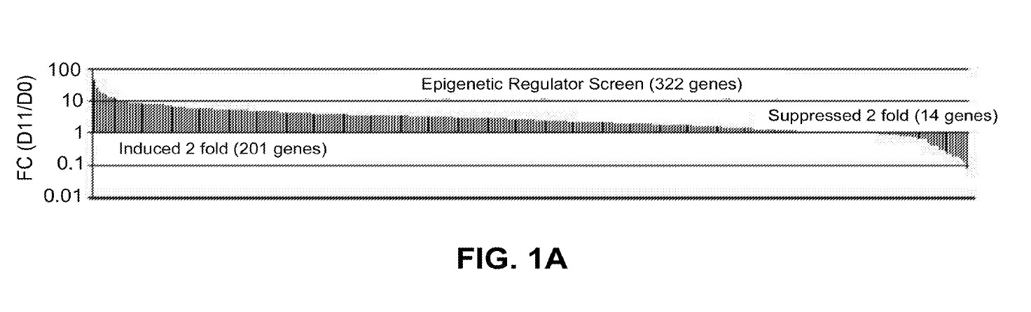

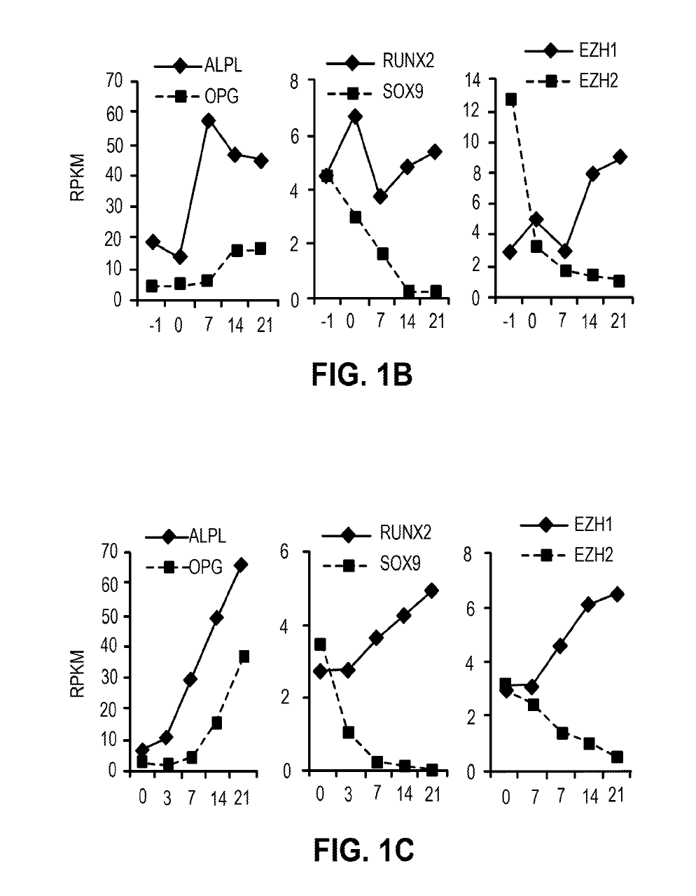

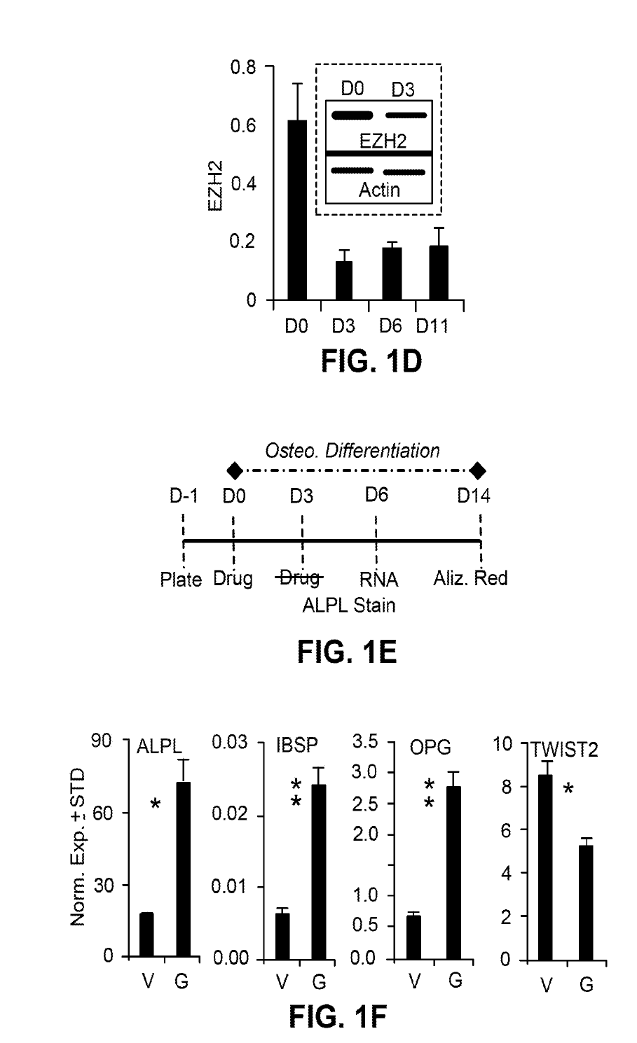

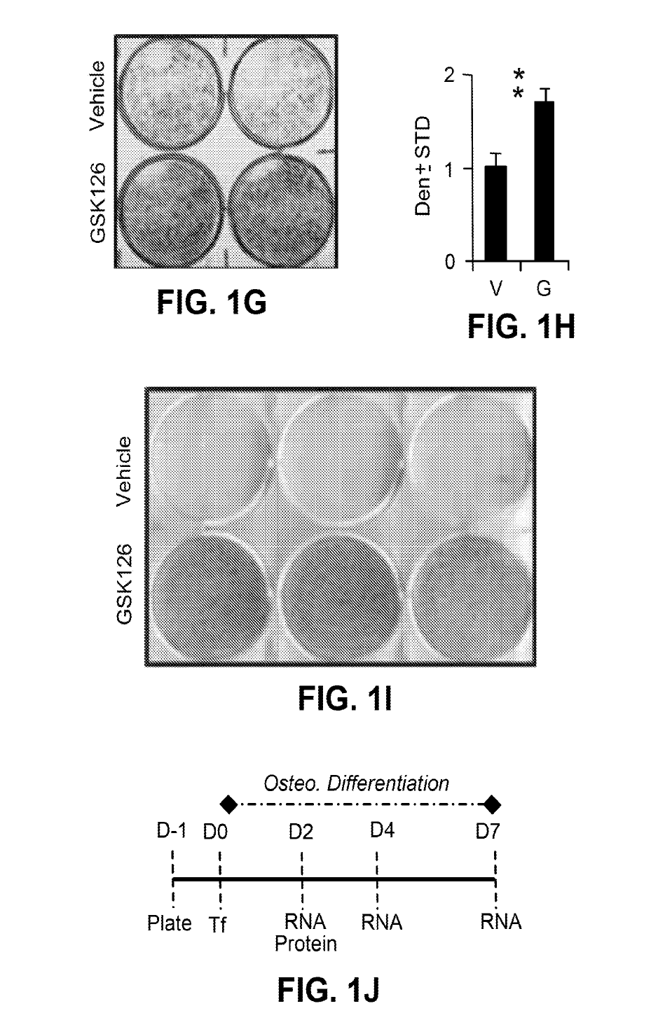

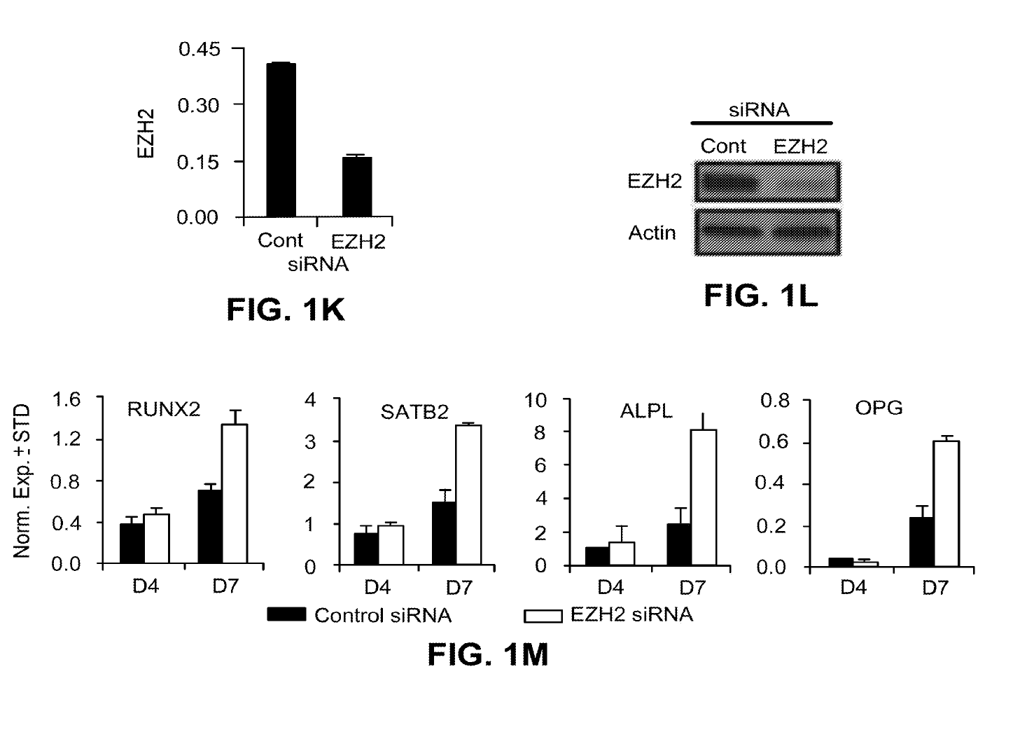

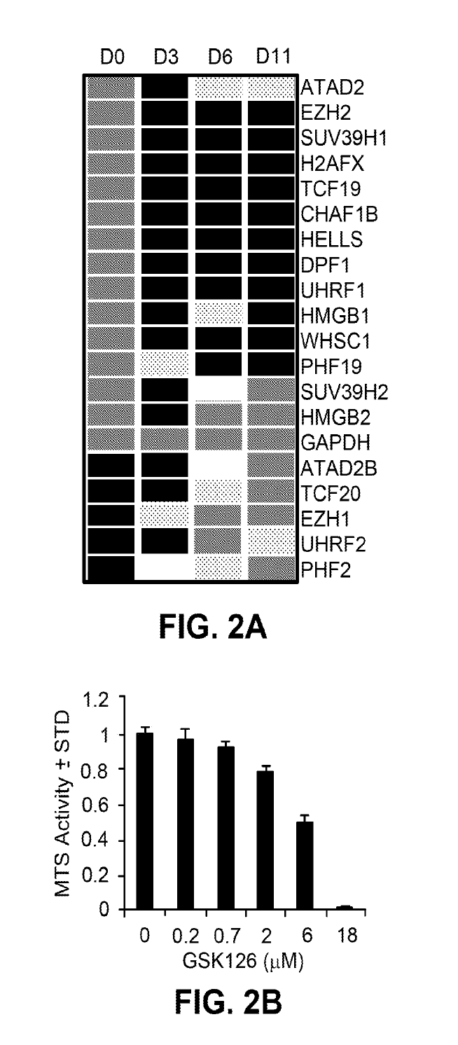

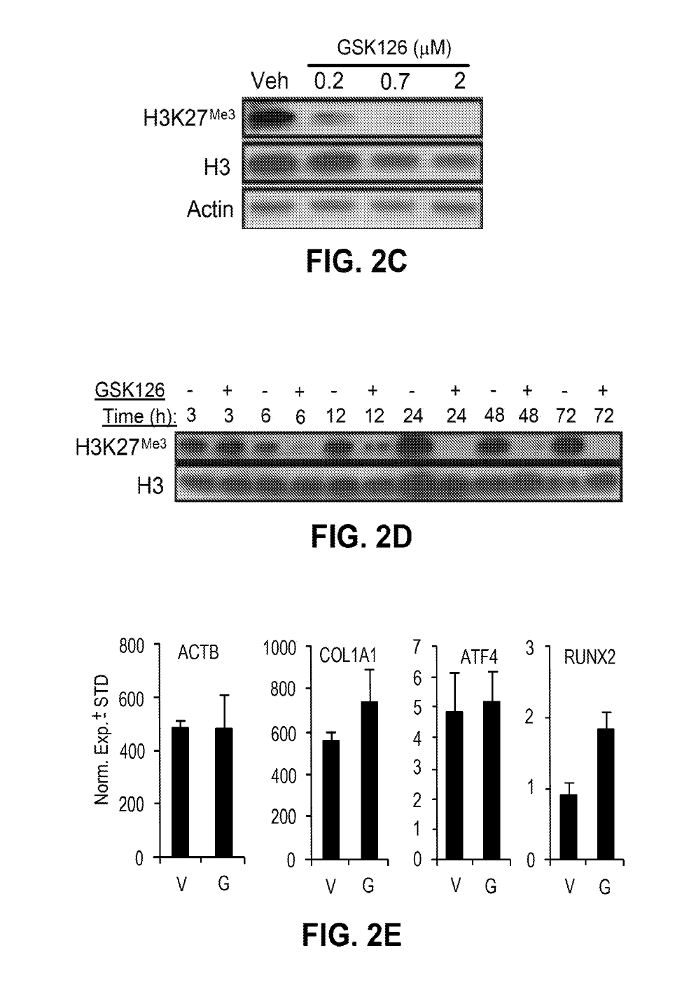

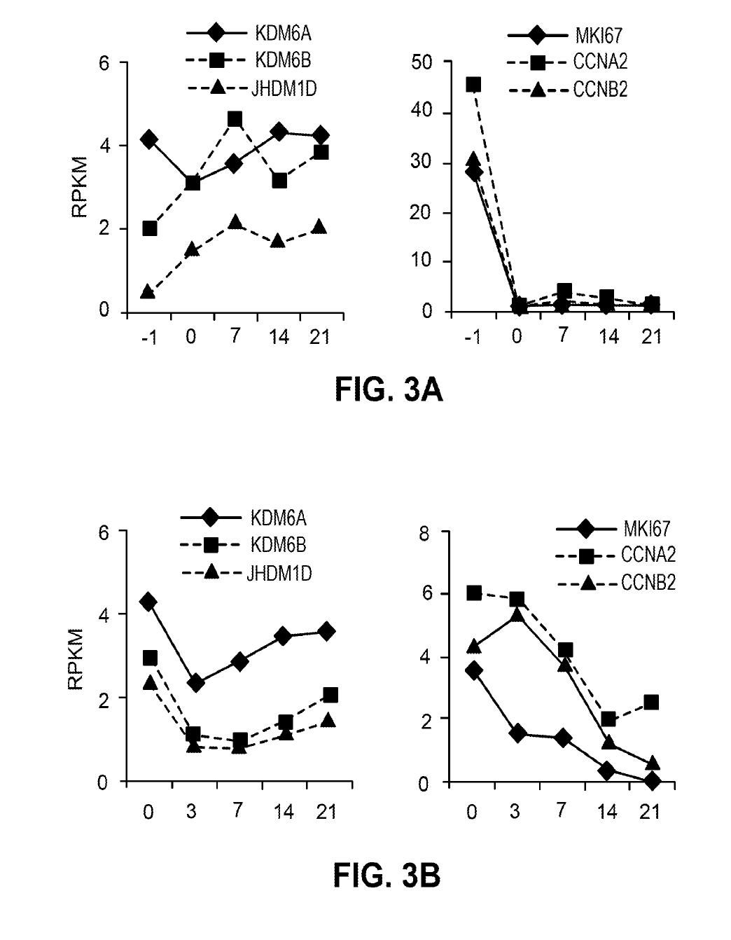

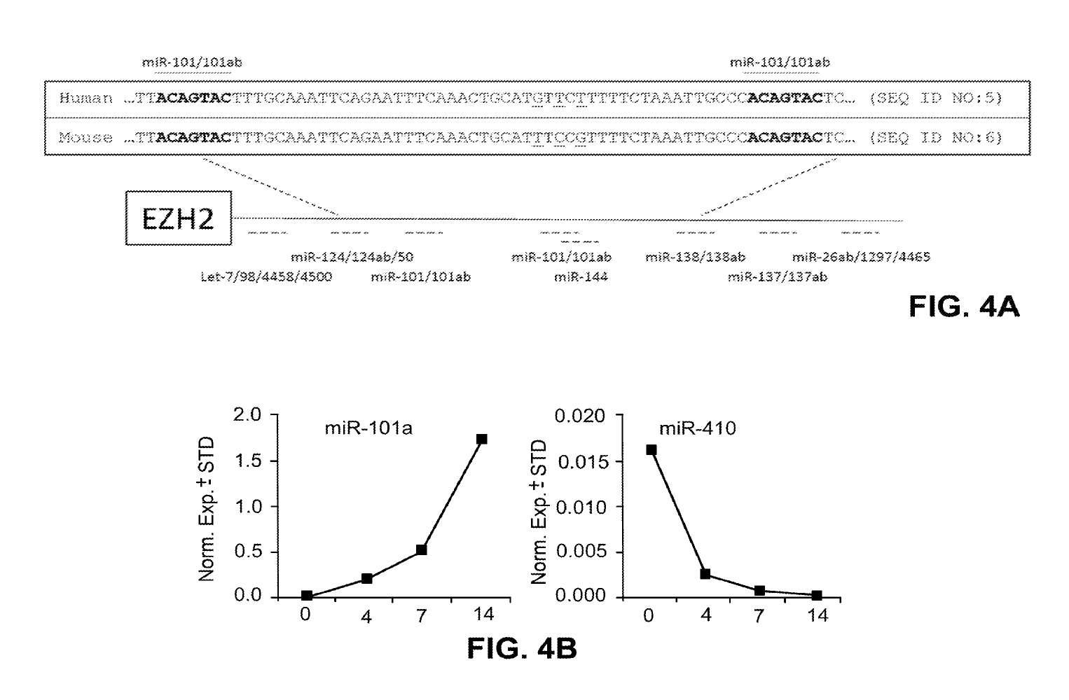

An mRNA expression screen was performed using RT-qPCR for more than 300 histone code writers, readers and erasers (Liu et al., Bioinformatics, 28:2205-2206 (2012)) in clinical-grade mesenchymal stromal cells (MSCs)(Dudakovic et al., J. Cell Biochem., 115:1816-1828 (2014) and Dudakovic et al., J. Cell Physiol., 230:52-62 (2015)) upon induction of osteogenic differentiation (FIG. 1A). Many epigenetic proteins (n=201) were up-regulated, but only 14 factors were down-regulated by at least 2 fold during osteogenic differentiation (FIGS. 1A and 2A). One prominently downregulated mRNA encoded the epigenetic regulator EZH2, the enzymatic subunit of the polycomb-repressive complex 2 (PRC2) that regulates chromatin accessibility by catalyzing mono-, di-, and tri-methylation of histone 3 (H3) lysine 27 (K27) (Czermin et al., Cell, 111:185-196 (2002); Muller et al., Cell, 111:197-208 (2002); Ebert et al., Genes Dev., 18:2973-2983 (2004); and Marchesi et al., Cell Cycle, 13:516-527 (2014)). Changes in EZH2 expression were validated by next-generation RNA sequencing (mRNASeq) performed on RNA samples of MSCs during osteogenic differentiation in two different cell culture conditions (FIGS. 1B, 1C, and 3). These changes in mRNA expression were confirmed by immuno-blotting, which revealed decreased EZH2 protein levels (FIG. 1D). EZH2 was transcriptionally suppressed by the bone master regulator Runx2 (Wu et al., Genome Biol., 15:R52 (2014)), a known target of miR-101 that is up-regulated in osteoblasts (FIG. 4A) and an epigenetic enzyme with focused activity that modifies a single histone residue (H3K27). Therefore, EZH2 was considered a promising target for development of bone anabolic strategies. In addition, it may enhance osteogenic lineage-commitment of MSCs (Margueron et al., Mol. Cell., 32:503-518 (2008); Wyngaarden et al., Development, 138:3759-3767 (2011); Wei et al., Nat. Cell Biol., 13:87-94 (2011); Zhu and Xu, Biochem. Biophys. Res. Commun., 432:612-617 (2013); Hui et al., J. Endod., 40:1132-1138 (2014); Hemming et al., Stem Cells, 32:802-815 (2014); and Schwarz et al., Development, 141:867-877 (2014)) and/or the maturation of pre-osteoblasts into cells that actively produce a mineralizing extracellular matrix (ECM).

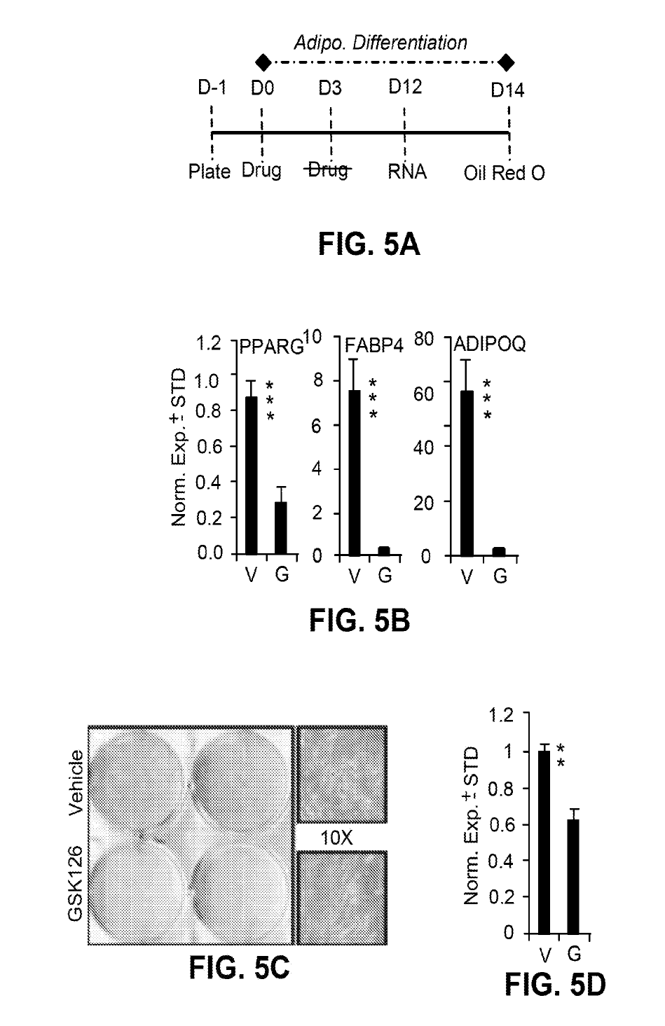

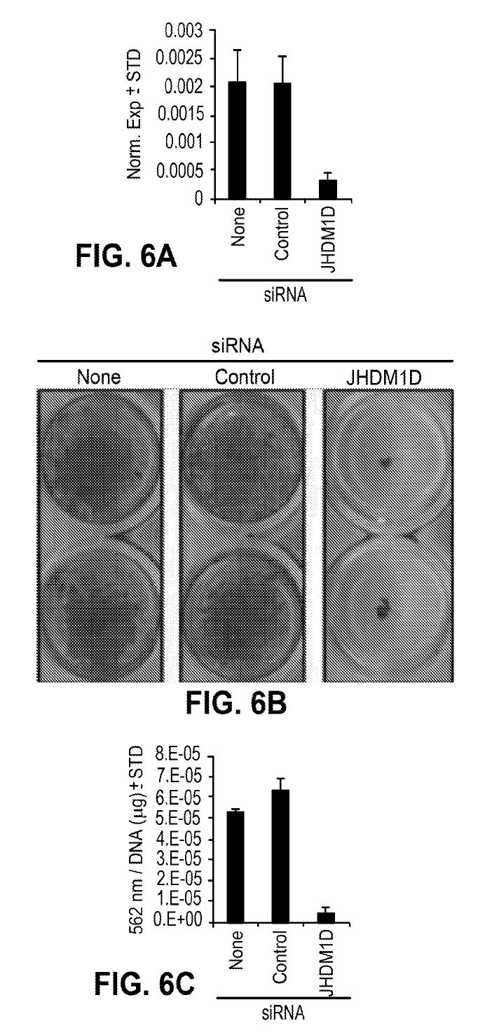

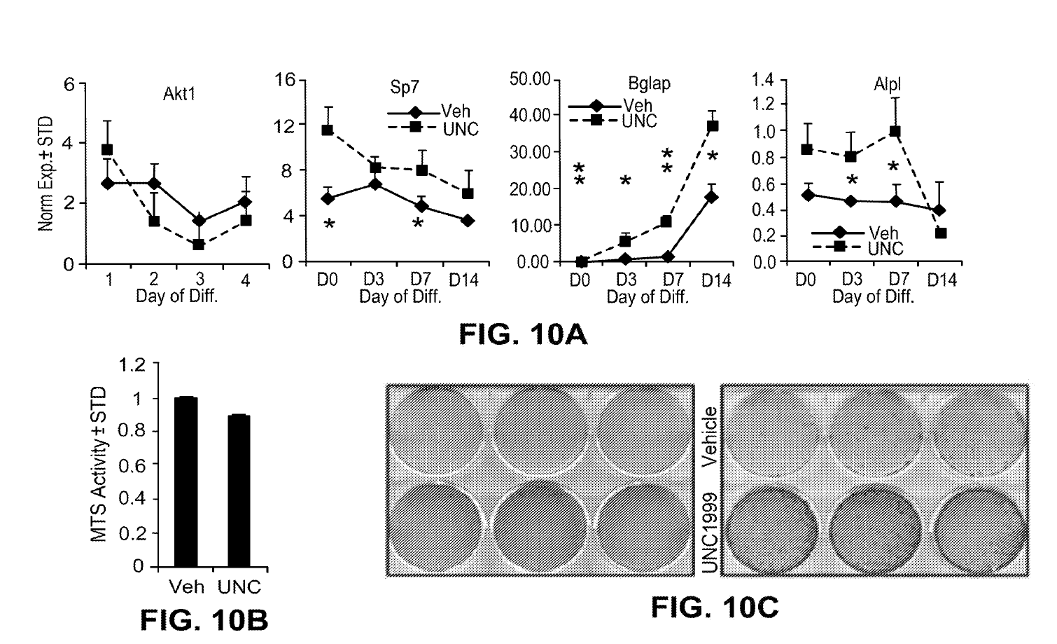

Pharmacologic inhibition of EZH2 evoked both a rapid and sustained response. H3K27 tri-methylation was attenuated as early as six hours after drug administration, and inhibition persisted for at least 72 hours at a non-cytotoxic dose (FIGS. 2B-2D). Thus, EZH2 was a principal mediator of global H3K27 tri-methylation (H3K27me3) levels in MSCs (Margueron et al., Mol. Cell., 32:503-518 (2008)). Pharmacological inhibition of EZH2 enhanced osteogenic differentiation of MSCs in the presence of media supplements that supported osteoblast differentiation (FIGS. 1E-1I and 2E), but these effects were not observed in the absence of differentiation cocktail. These stimulatory effects were evidenced by elevated expression of established osteoblastic mRNA markers (FIG. 1F), increased staining for alkaline phosphatase (FIGS. 1G and 1H) and alizarin red (FIG. 10. The same biological effects were observed when MSCs were treated with a small-interfering RNA (siRNA) for EZH2 over two days (FIGS. 1J-1M). Treatment of MSCs with EZH2 inhibitors also significantly reduced adipogenic differentiation (FIG. 5). Thus, inhibition of the H3K27 methyl transferase EZH2 preferentially promoted osteogenic and inhibited adipogenic lineage-commitment of mesenchymal progenitor cells. Furthermore, siRNA inhibition of the H3K27 demethylase JHDM1D (which opposes K27 methylation) blocked osteogenic differentiation of MSCs (FIG. 6), consistent with a model in which selective reduction of H3K27 methylation enhances osteogenic differentiation.

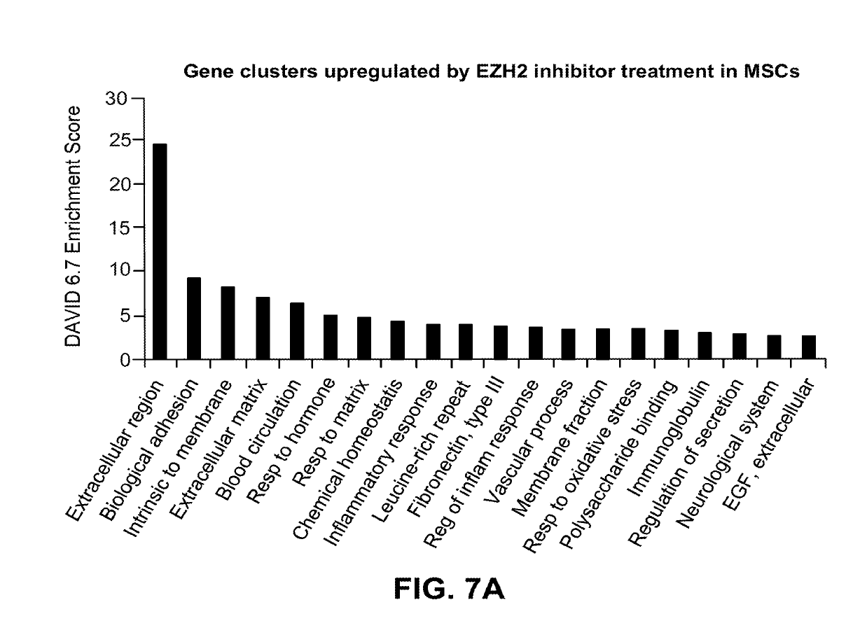

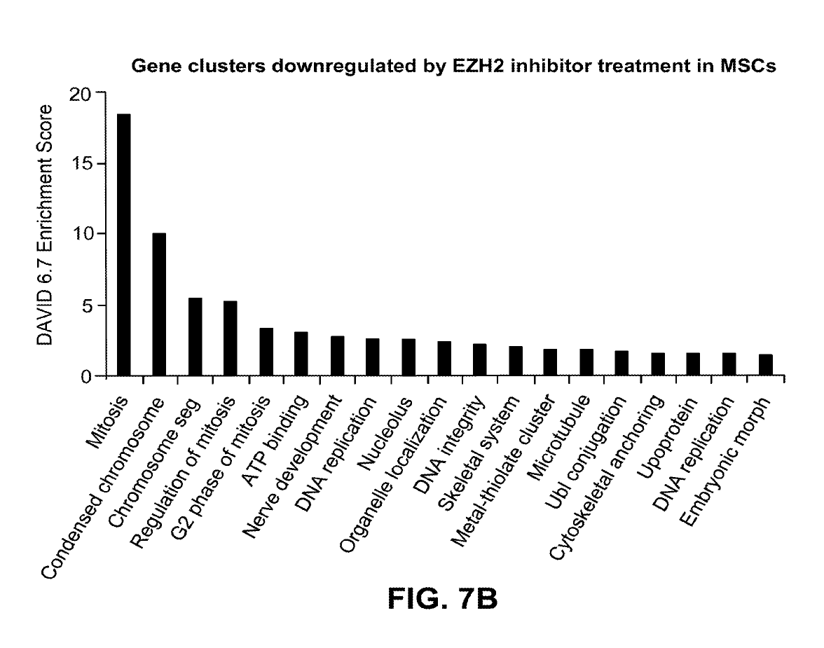

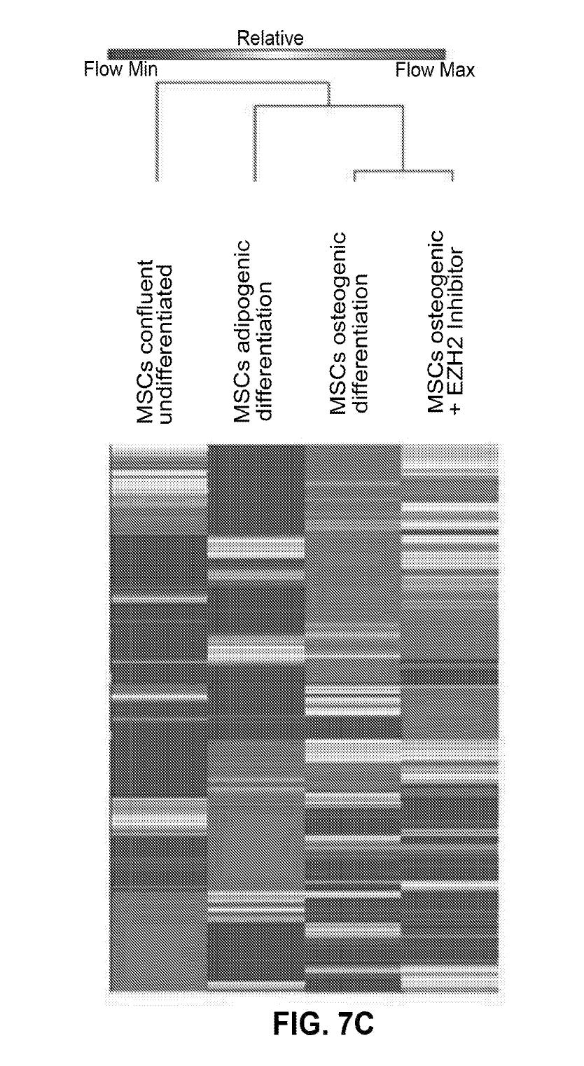

Analysis of MSCs treated by pharmacologic inhibitors by mRNASeq was performed to identify molecular pathways altered by EZH2 inhibition. Gene ontology analysis demonstrated that EZH2 inactivation primarily stimulated expression of genes encoding extracellular matrix proteins (FIG. 7A), while suppressing genes involved in cell cycle progression and microtubule-processing (FIG. 7B). Hence, EZH2 inhibition of MSCs reinforced a non-proliferative and ECM-anabolic biological state. Furthermore, EZH2 inhibition induced genes encoding a large number of transcriptional regulators involved in osteogenic cell fate commitment and lineage progression (FIG. 7C), thus potentially clarifying why EZH2 inhibition enhanced osteogenic differentiation and inhibited adipogenic lineage commitment of MSCs. Thus, MSC lineage commitment was affected in part by direct and indirect alterations of EZH2 dependent gene regulatory programs.

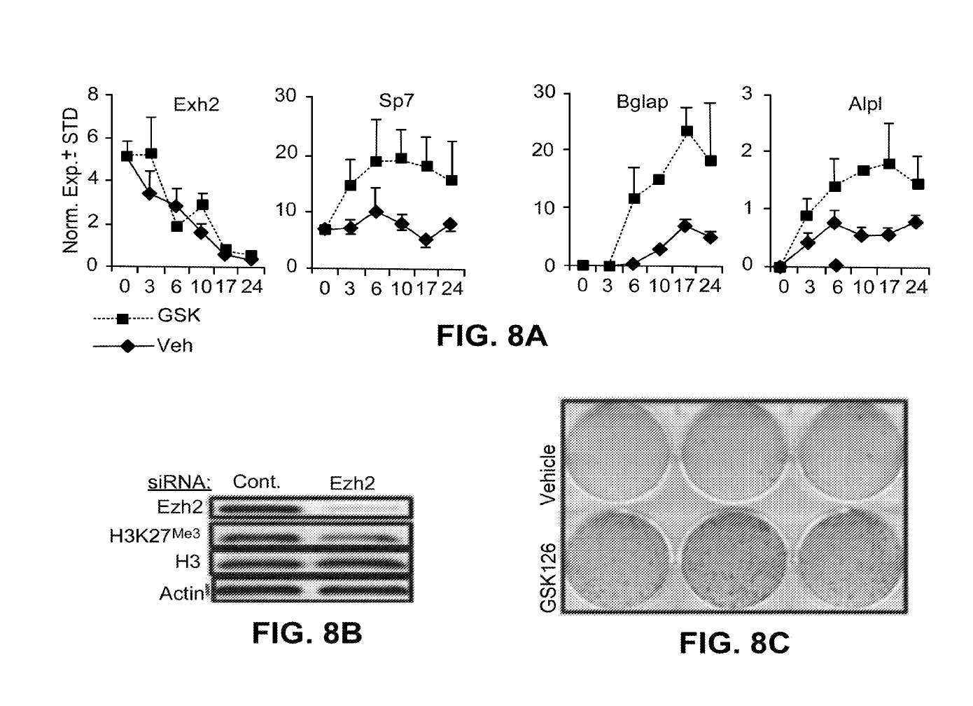

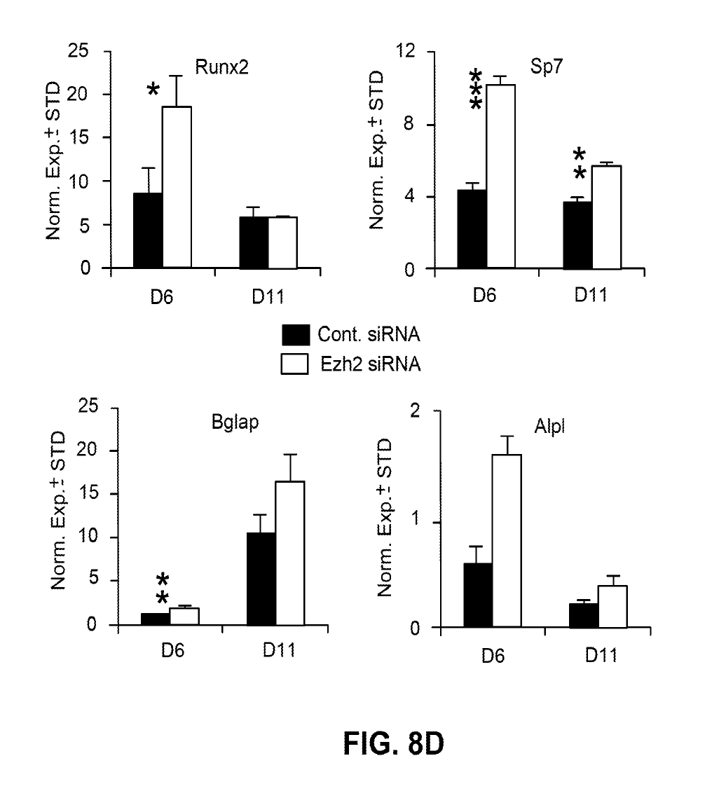

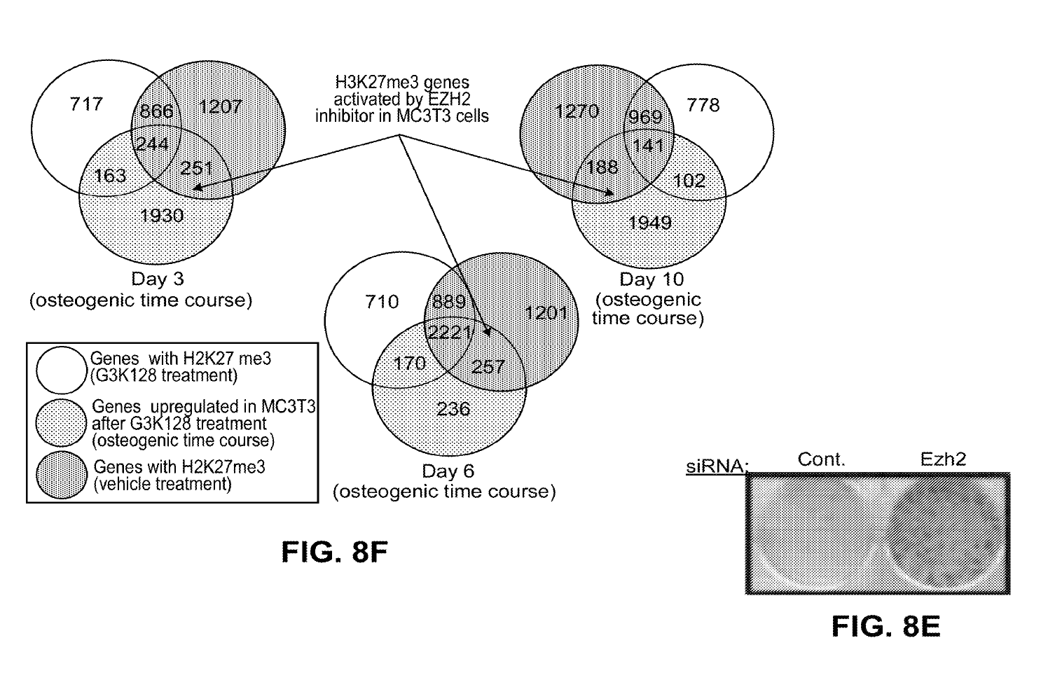

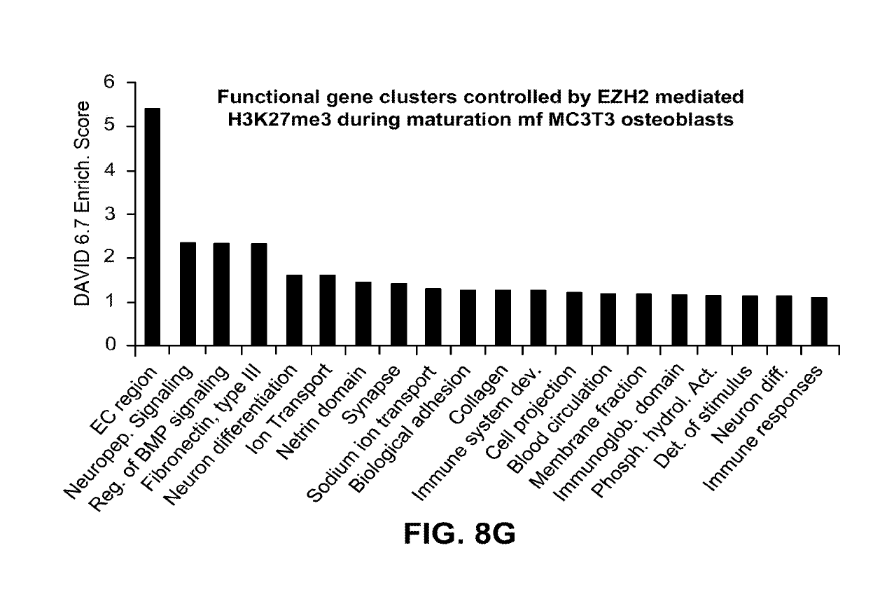

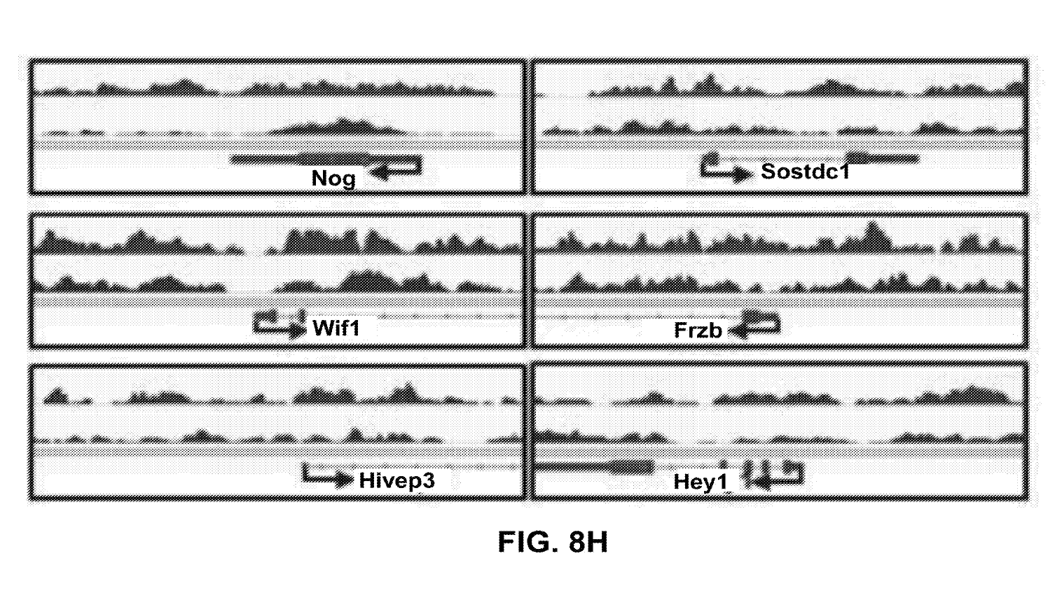

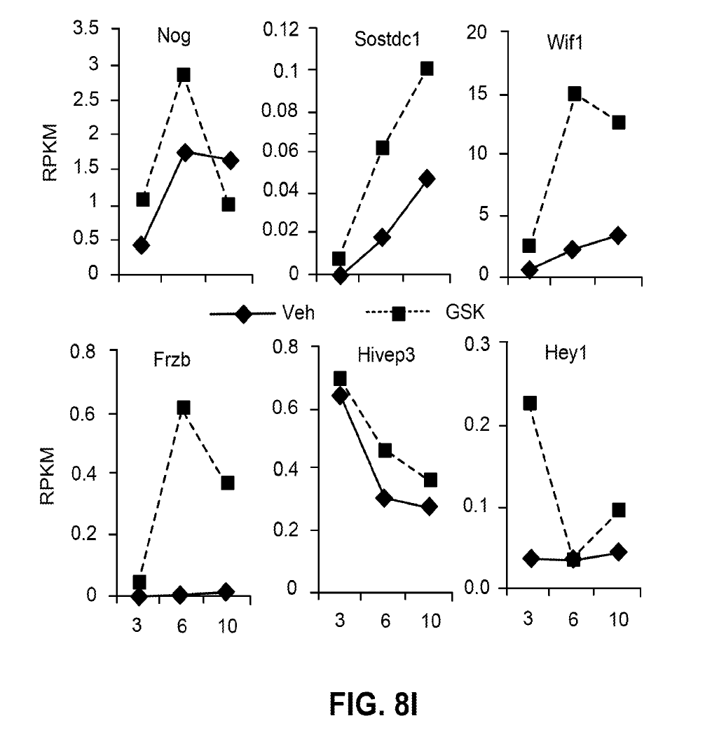

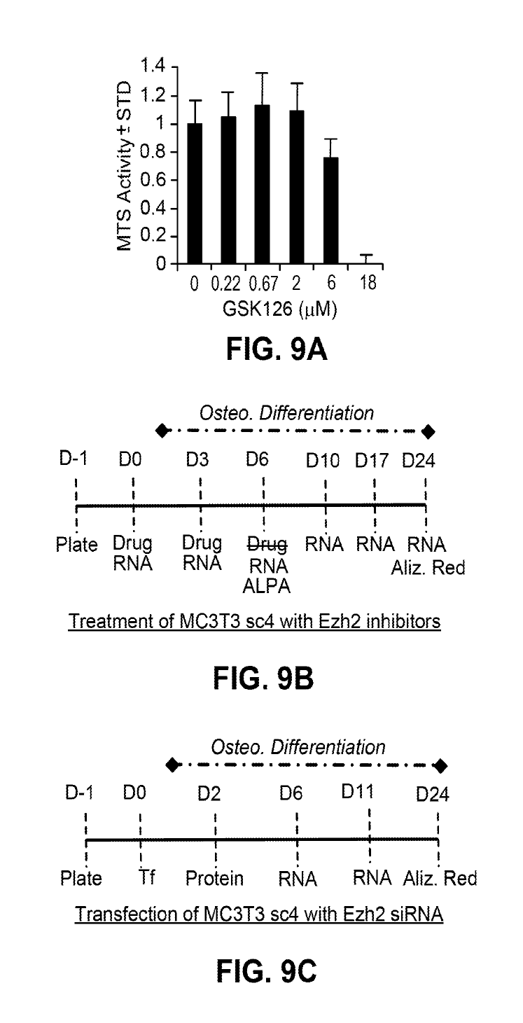

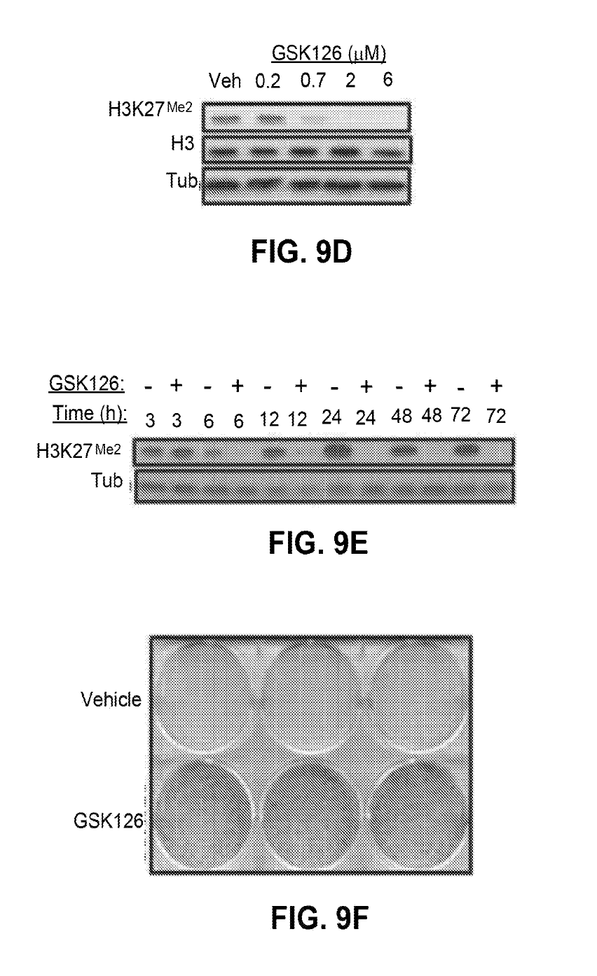

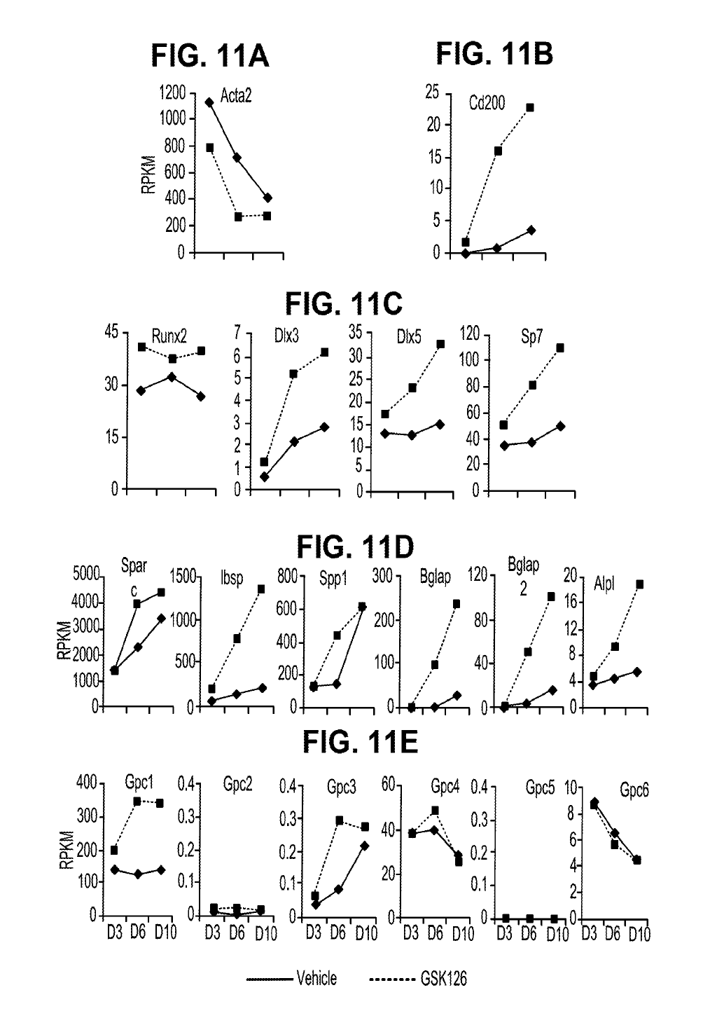

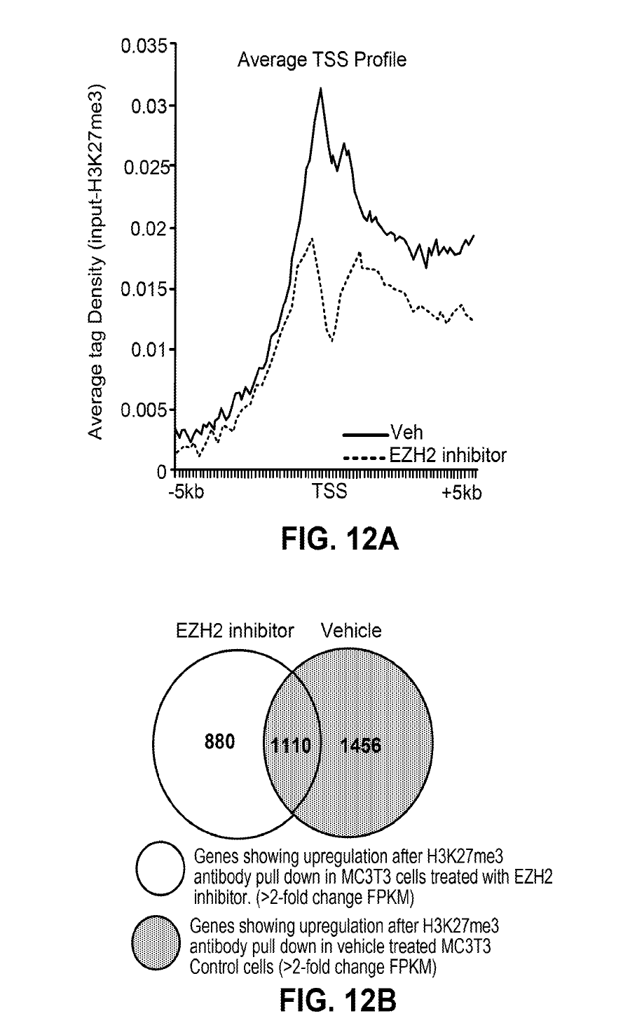

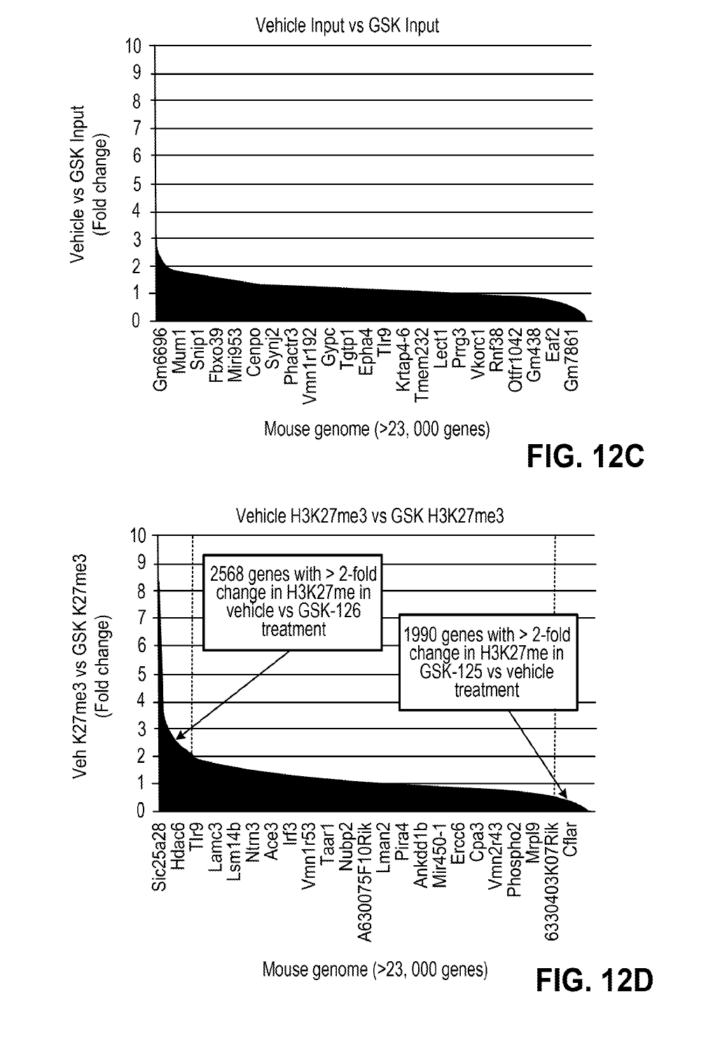

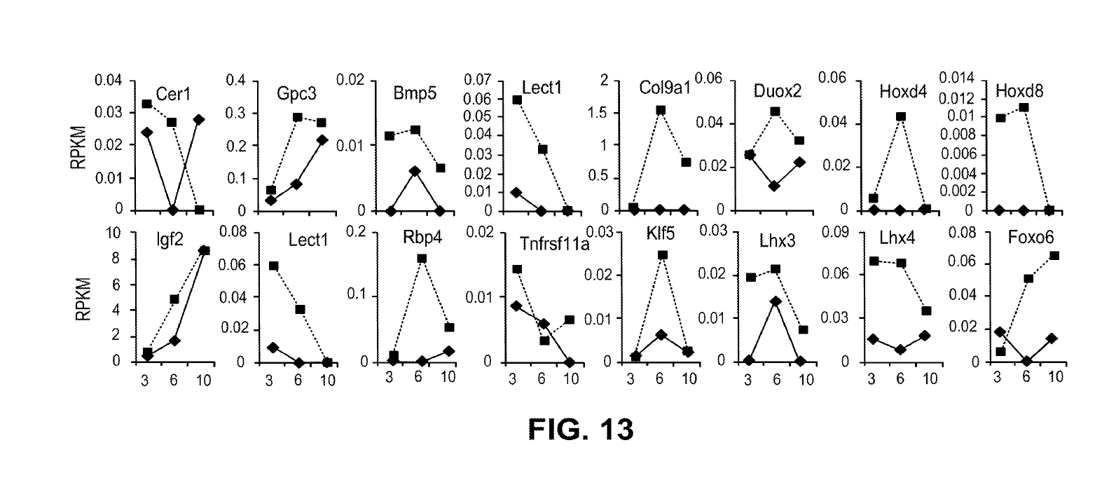

Osteoblastogenesis during bone formation required both osteogenic lineage-commitment of MSCs and subsequent maturation of pre-osteoblastic progenitors. Therefore, EZH2 inhibition using siRNA and pharmacologic inhibitors was tested to determine effects on lineage-progression of pre-committed osteoblastic cells. Similar to the studies on osteogenic cell fate determination with MSCs (FIG. 1), decreased EZH2 activity by protein depletion or enzymatic inhibition was found to expedite osteoblast maturation (FIGS. 8A-8E, 9, and 10). Phenotypic examination of osteoblast maturation by ChIPSeq and mRNASeq analysis revealed that EZH2 inhibition results in H3K27 demethylation and subsequent upregulation of a number of genes with H3K27me3 marks for key osteogenic transcription factors, growth factors, and genes that modulate BMP signaling (FIGS. 8F, 8G, and 11). Mechanistically, enzymatic inhibition of EZH2 using GSK126 decreased the deposition of H3K27me3 marks near transcriptional start sites (TSSs) across the genome, based on chromatin immuno-precipitation analysis combined with next-generation DNA sequencing (ChIPSeq) (FIGS. 8H, 8I, 12, and 13).

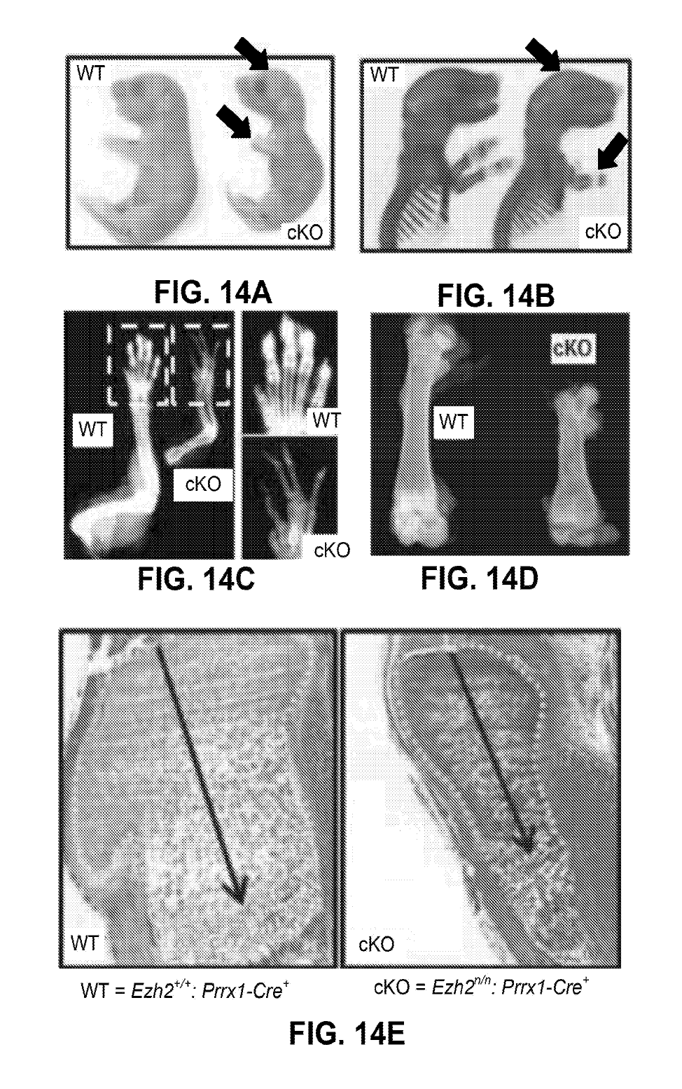

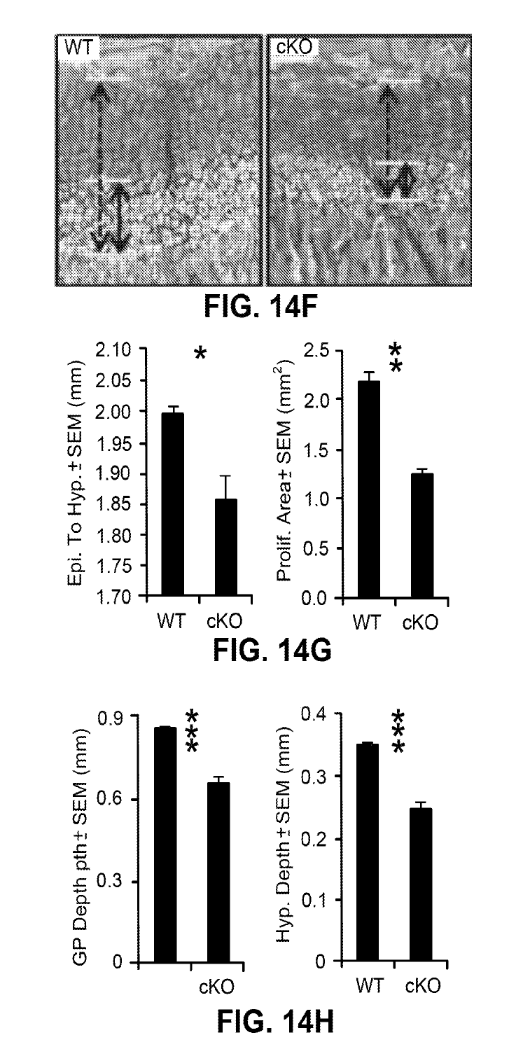

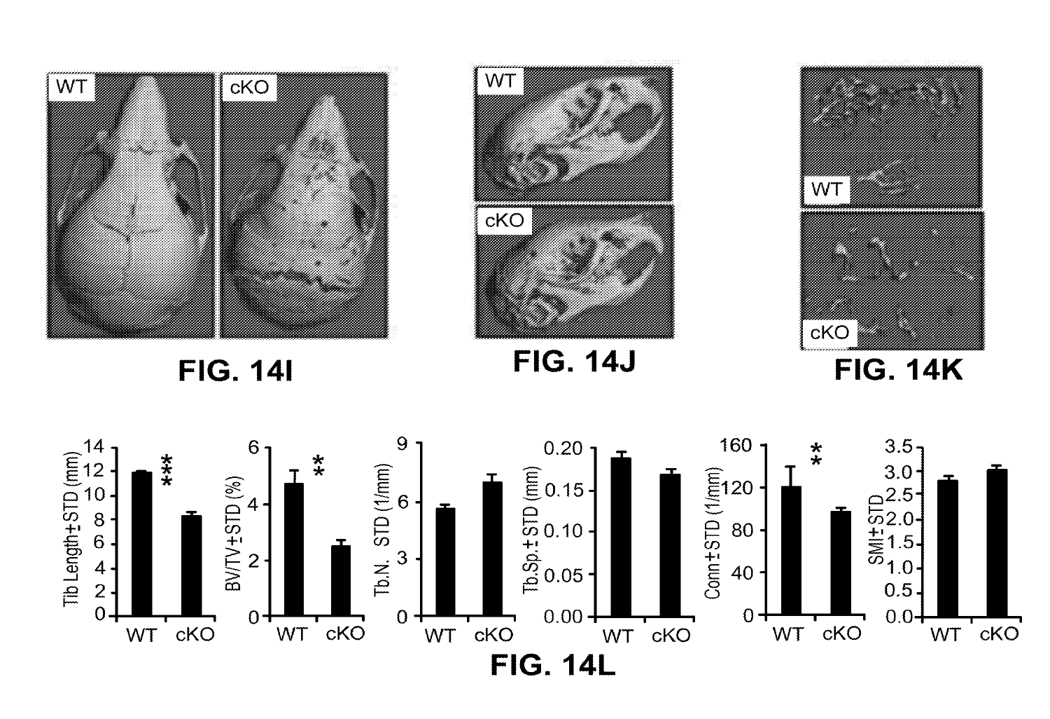

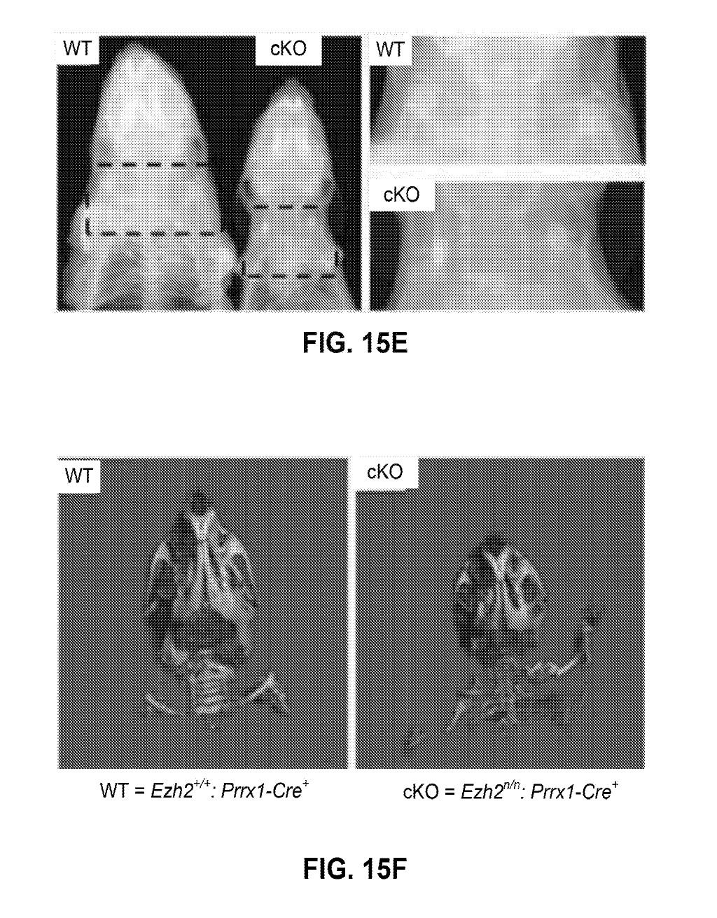

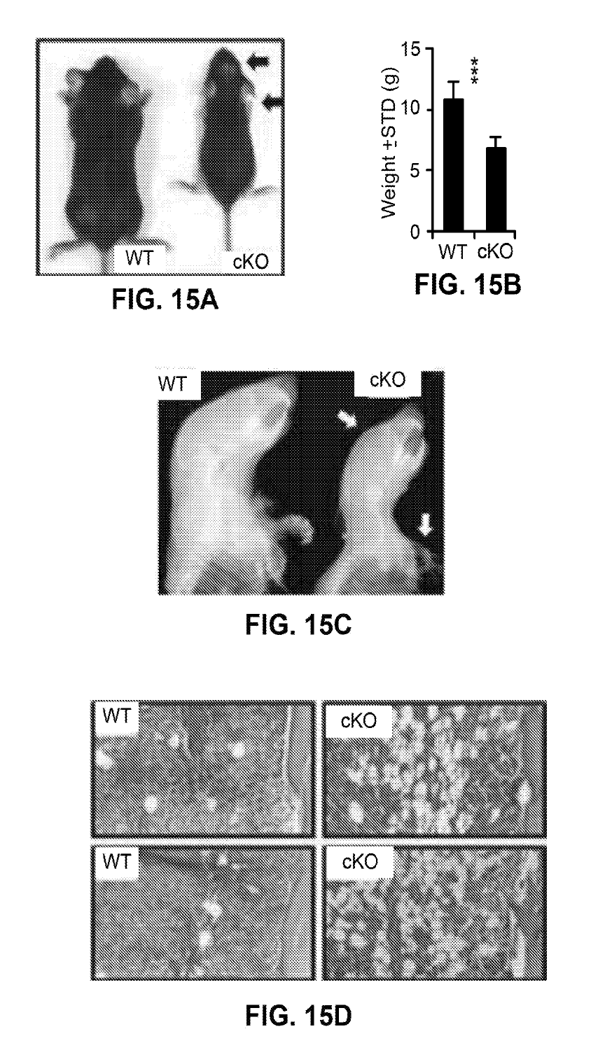

The epigenetic function of EZH2 during skeletal development and mesenchymal cell differentiation in vivo was initially examined using a conditional knock-out mouse model. Mice with a constitutive Ezh2 null mutation perish during early embryonic development (O'Carroll et al., Mol. Cell Biol., 21:4330-4336 (2001)). However, ‘Ezh2-cKOPrrx1’ mice, which contain a conditional Ezh2fl/fl allele (Su et al., Nat. Immunol., 4:124-131 (2003)) that is ablated in uncommitted mesenchymal cells (Logan et al., Genesis, 33:77-80 (2002)), survive until weaning and have a reduced body size in comparison with wild type (WT) mice (FIGS. 14A, 15A, and 15B). The smaller body size of Ezh2-cKOPrrx1 mice was reflected by differences in the size of the front limbs and tibial length in hind legs, as well as decreased spine length and the height of individual vertebrae (FIGS. 14B-14D). Furthermore, Ezh2-cKOPrrx1 mice exhibited multiple skeletal abnormalities doming of the head and premature closure of the cranial sutures, which were characteristic of craniosynostosis (FIGS. 14 and 15). They also demonstrated clinodactyly, which was reminiscent of patients with Weaver syndrome, a disorder known to be associated with germline mutations in EZH2 (FIGS. 14C and 15C). Loss of Ezh2 in the mesenchymal progenitor compartment also resulted in growth plate abnormalities (FIGS. 14E-14H and 15D). Growth plate related perturbations of endochondral ossification may account for the observed decrease in the size of the limbs and vertebrae that resulted in the shorter stature of the mice.

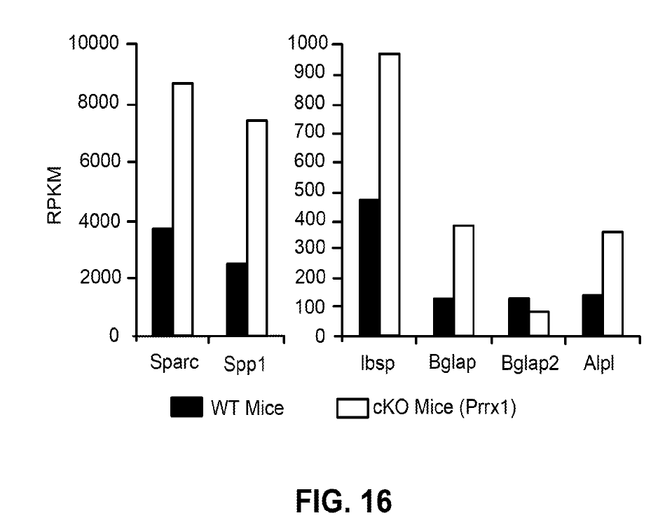

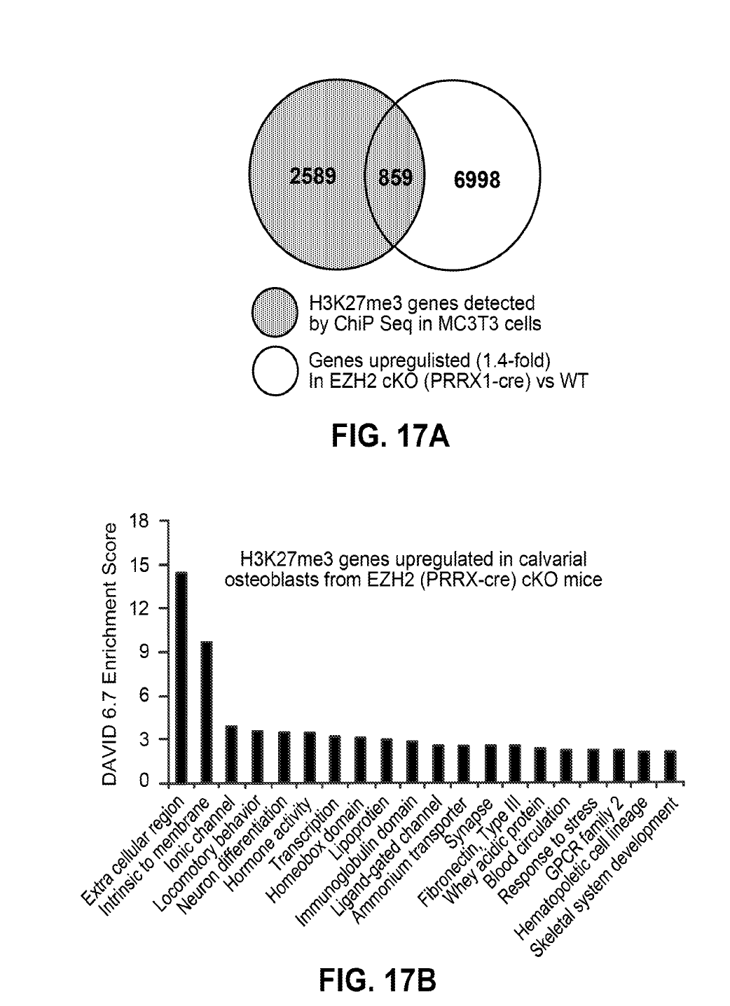

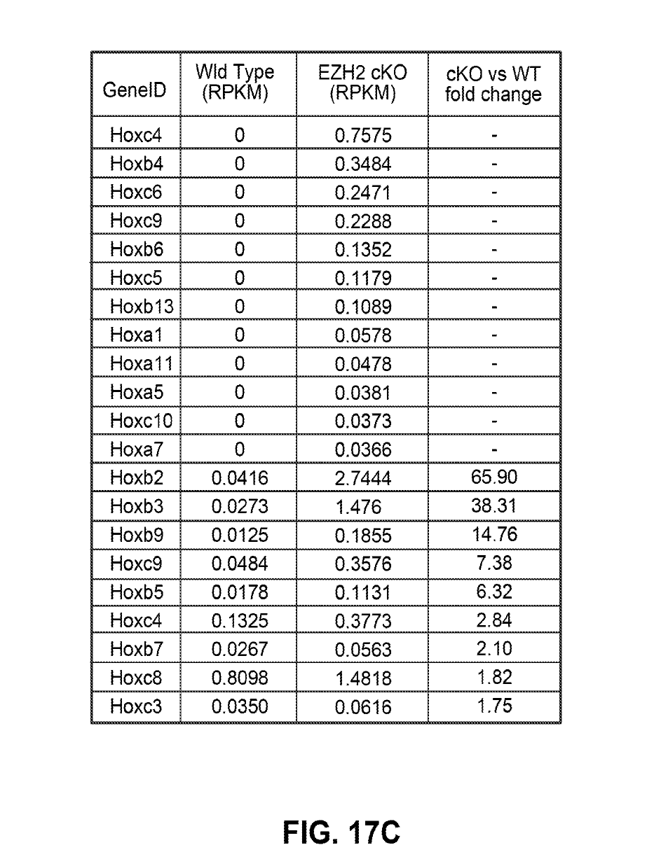

Abnormalities in cranial development in Ezh2-cKOPrrx1 mice reflected a perturbation in membranous bone formation. To understand the molecular mechanisms by which EZH2 loss in cranial osteogenic precursor cells accelerated suture closure, mRNASeq analysis of mRNAs from calvarial cells of Ezh2-cKOPrrx1 and WT mice was performed (FIGS. 16 and 17). Loss of EZH2 in calvarial cells increased expression of several bone-related ECM proteins as expected from precocious maturation of pre-osteoblastic progenitors. Among the most prominently unregulated non-ECM genes were the known Ezh2 targets gene CDKN2A (Cakouros et al., Mol. Cell. Biol., 32:1433-1441 (2012)), encoding the senescence-associated cyclin-dependent kinase (CDK) inhibitory protein p16, and CDKN1C (Yang et al., PLoS One, 4:e5011 (2009)), which generates the CDK inhibitor p57. Thus, EZH2 knockout in calvarial cells induced a post-proliferative state concomitant with increased production of a bone-related mineralizing ECM.

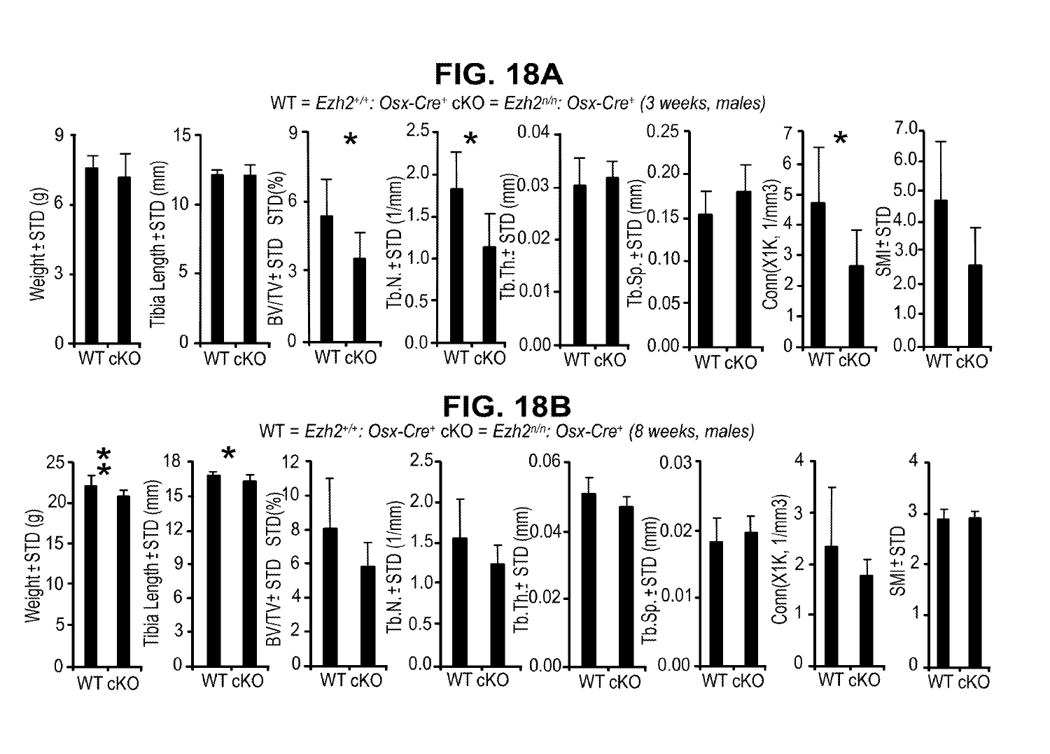

The physiological role of EZH2 in osteoprogenitor cell differentiation in vivo was examined using mice in which EZH2 is deleted in cells that express Cre under control of the promoter for the transcription factor Osterix (Osx)/Sp7 in committed pre-osteoblasts and hypertrophic chondrocytes (Rodda and McMahon, Development, 133:3231-3244 (2006)). These Ezh2-cKOOsx mice were normal in appearance, and initially exhibited a low bone mass phenotype at weaning that normalized during early adulthood (FIG. 18). These results were consistent with a reduction in the proliferative expansion of pre-osteoblast progenitors due to expedited acquisition of a post-proliferative differentiated state.

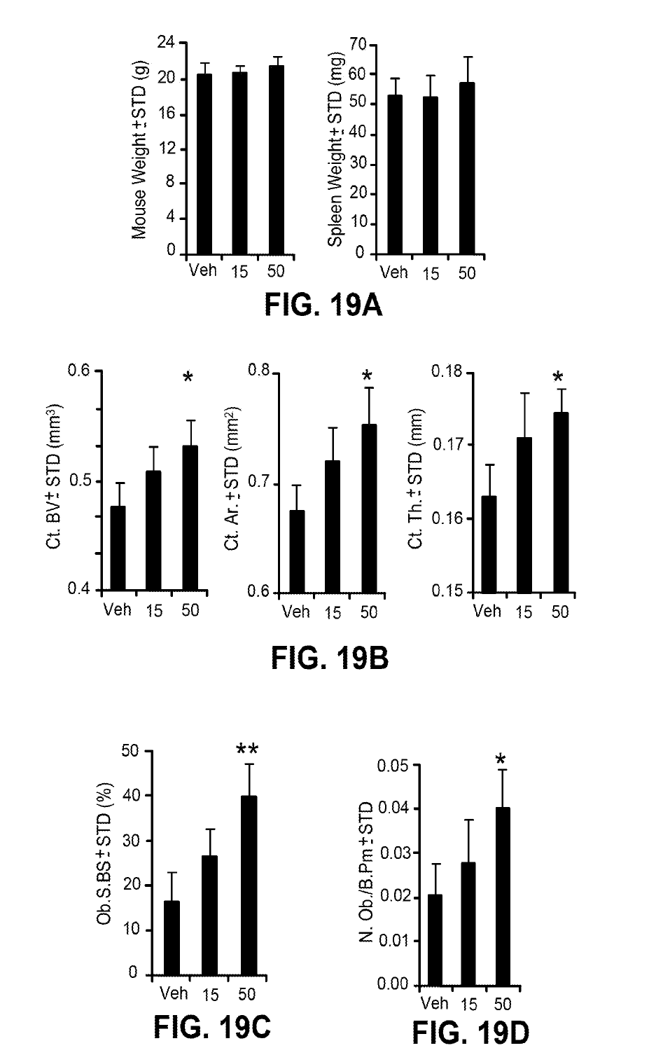

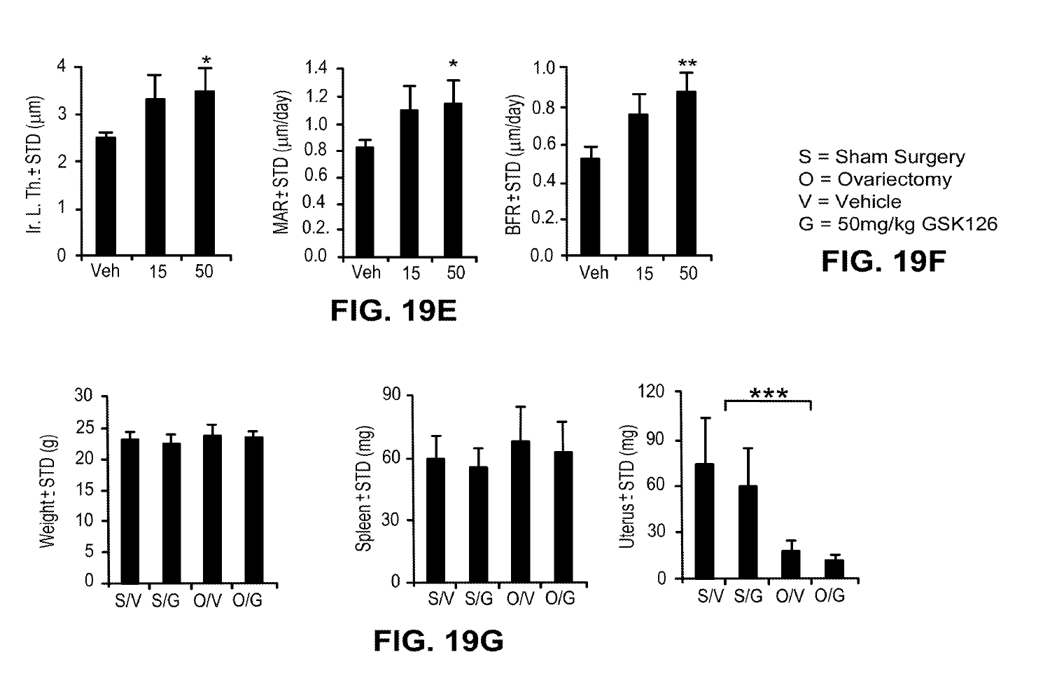

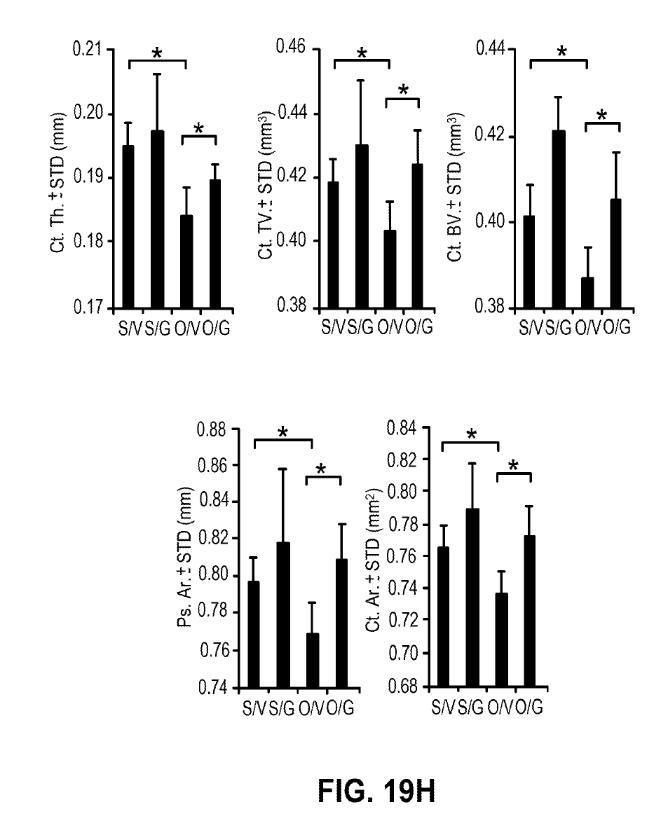

In addition to regulating early skeletal patterning and development in growing mice (FIGS. 14, 15, and 18), these results indicate that EZH2 has pro-osteogenic properties by enhancing the expression of bone ECM proteins. Thus, the biological effects of EZH2 was assessed during bone homeostasis in adult mice (>8 weeks) with a fully mature skeleton (FIG. 19). Daily administration of the EZH2 inhibitor GSK126 (at 50 mg/kg) for 5 weeks increased several cortical bone parameters, and thus pharmaceutical inactivation of EZH2 had bone-anabolic effects in adult mice (FIGS. 19A-19E and Tables 1-3). The loss of EZH2 function also was examined in female mice that exhibited estrogen-deficiency induced osteoporosis upon ovariectomy (OVX). Treatment of these mice with a daily regimen of GSK126 mitigated OVX-induced cortical bone loss, demonstrating that inhibition of EZH2 in adult females has osteoprotective properties (FIGS. 19F-19H, and Tables 4 and 5).

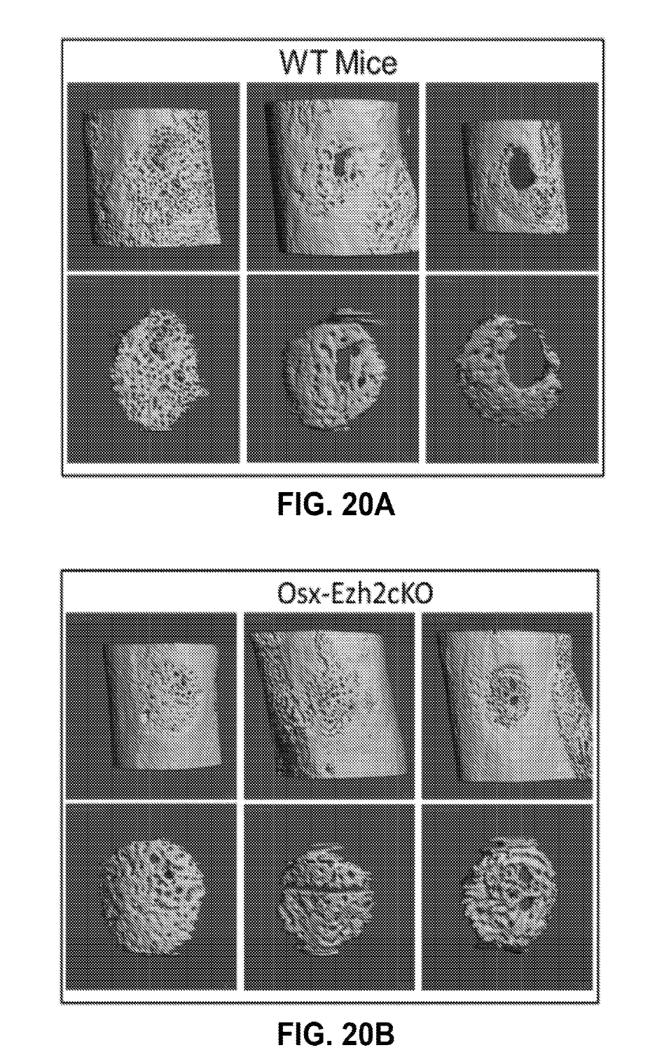

Additional studies demonstrated that deletion of functional Ezh2 enhances fracture healing in mice. Intramembranous bone healing was assessed with a femoral drill-hole model in 8 week old wild type mice (WT; FIG. 20A) and in animals in which functional Ezh2 was ablated in osteoblasts (Osx-Ezh2cKO; FIG. 20B). To induce a single-cortex defect in the femur, mice were incised above the mid-diaphysis of the femur to expose the bone, and a 0.7 mm diameter steel burr drill bit was used to create the defect. Defect healing was assessed by micro-CT 14 after surgery. These studies demonstrated that deletion of Ezh2 in the osteoblast lineage enhanced bone healing in adult animals.

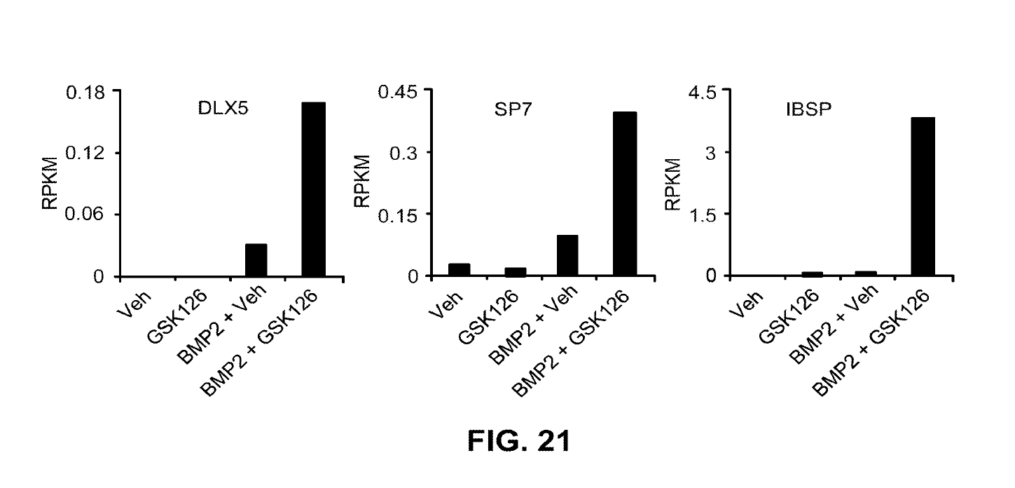

In addition, osteogenic genes are synergistically activated by GSK126 and BMP2. Mesenchymal stem cells were treated with 50 ng/ml BMP2 and/or 2 μM GSK126. RNA-Seq analysis demonstrated that Ezh2 inhibition (GSK126) and BMP2 activated different set of genes, but they synergistically enhanced the expression of key osteogenic factors such as DLXS (FIG. 21, left panel), SP7 (FIG. 21, center panel), and IBSP (FIG. 21, right panel). Thus, EZH2 inhibition enhances the osteogenic potential of cells treated with BMP2, a key osteogenic activator.

TABLE 1
Trabecular bone architectural properties in the distal femur metaphysis
was not significantly altered with GSK126 administration.

| | | GSK126 | | GSK126 | |
|---|---|---|---|---|---|
| Property | Vehicle | (15 mg/kg) | p-value | (50 mg/kg) | p-value |
| BV/TV, % | 0.077 | 0.072 | 0.763 | 0.083 | 0.664 |
| | (0.005) | (0.013) | | (0.029) | |
| Tb.N, mm⁻¹ | 4.087 | 3.850 | 0.497 | 4.053 | 0.818 |
| | (0.088) | (0.304) | | (0.106) | |
| Tb.Th, mm | 0.040 | 0.040 | 0.758 | 0.043 | 0.231 |
| | (0.001) | (0.002) | | (0.002) | |
| Tb.Sp, mm | 0.246 | 0.267 | 0.434 | 0.252 | 0.471 |
| | (0.005) | (0.023) | | (0.005) | |

Vehicle n = 3, GSK126 15 mg/kg n = 3, GSK126 50 mg/kg n = 3;
Means (standard error) are presented

TABLE 2
GSK126 administration significantly increases cortical
bone properties of the femoral midshaft.

| | | GSK126 | | GSK126 | |
|---|---|---|---|---|---|
| Property | Vehicle | (15 mg/kg) | p-value | (50 mg/kg) | p-value |
| Ct. TV, mm³ | 1.146 | 1.184 | 0.467 | 1.243 | 0.120 |
| | (0.024) | (0.041) | | (0.043) | |
| Ct. BV, mm³ | 0.477 | 0.509 | 0.157 | 0.532 | 0.042 |
| | (0.013) | (0.013) | | (0.014) | |
| Ct. Th, mm | 0.163 | 0.171 | 0.148 | 0.175 | 0.021 |
| | (0.003) | (0.004) | | (0.002) | |
| Ct. Mat.M.Dn, | 1242 | 1235 | 0.282 | 1238 | 0.196 |
| mgHA/cm³ | (0.357) | (5.130) | | (2.170) | |
| Ps.Ar, mm² | 1.620 | 1.675 | 0.438 | 1.758 | 0.105 |
| | (0.024) | (0.058) | | (0.061) | |
| Ct. Ar, mm² | 0.674 | 0.719 | 0.126 | 0.753 | 0.032 |
| | (0.014) | (0.019) | | (0.020) | |
| Ec. Ar, mm² | 0.947 | 0.955 | 0.865 | 1.006 | 0.245 |
| | (0.010) | (0.047) | | (0.042) | |

Vehicle n = 3, GSK126 15 mg/kg n = 3, GSK126 50 mg/kg n = 3;
Means (standard error) are presented;
p < 0.05

TABLE 3
Static histomorphometry in the distal femoral metaphysis.

| | | N.Oc/B.Pm, | | | N.Ob/B.pm, | |
|---|---|---|---|---|---|---|
| Group | Oc.S/BS,% | #/mm | N.Oc/T.Ar | Ob.S/BS, % | #/mm | N.Ob/T.Ar |
| Vehicle | 0.195 | 0.016 | 0.058 | 0.590 | 0.020 | 0.071 |
| | (0.021) | (0.006) | (0.031) | (0.256) | (0.008) | (0.044) |
| GSK126 - | 0.314 | 0.015 | 0.042 | 0.354 | 0.028 | 0.109 |
| 15 mg/kg | (0.179) | (0.007) | (0.009) | (0.149) | (0.010) | (0.056) |
| GSK126 - | 0.240 | 0.018 | 0.050 | 0.551 | 0.040 | 0.081 |
| 50 mg/kg | (0.044) | (0.003) | (0.005) | (0.032) | (0.009) | (0.031) |
| p-value | 0.231 | 0.823 | 0.444 | 0.239 | 0.381 | 0.409 |
| (15 mg/kg) | | | | | | |
| p-value | 0.108 | 0.549 | 0.608 | 0.740 | 0.019 | 0.712 |
| (50 mg/kg) | | | | | | |

Vehicle n = 4,
GSK126 15 mg/kg n = 3,
GSK126 50 mg/kg n = 5;
Means (standard deviation) are presented;
p < 0.05

TABLE 4
GSK126 administration significantly increases cortical bone properties of the
femoral midshaft in an estrogen-deficient model.

| | SHAM | | | OVX | | |
|---|---|---|---|---|---|---|
| Property | Vehicle | GSK126 | p-value | Vehicle | GSK126 | p-value |
| Ct. TV, mm³ | 0.418 | 0.430 | 0.310 | 0.404 | 0.425 | 0.015 |
| | (0.004) | (0.009) | | (0.004) | (0.005) | |
| Ct. BV, mm³ | 0.402 | 0.411 | 0.377 | 0.387 | 0.405 | 0.028 |
| | (0.004) | (0.008) | | (0.003) | (0.005) | |
| Ct. Th, mm | 0.195 | 0.197 | 0.614 | 0.184 | 0.19 | 0.044 |
| | (0.002) | (0.004) | | (0.002) | (0.001) | |
| Ct. Mat.M.Dn, mgHA/cm³ | 1085 | 1065 | 0.006 | 1066 | 1067 | 0.985 |
| | (3.890) | (3.648) | | (5.726) | (4.015) | |
| Ct. Ps.Ar, mm² | 0.797 | 0.82 | 0.310 | 0.769 | 0.809 | 0.015 |
| | (0.007) | (0.018) | | (0.008) | (0.009) | |
| Ct. Ar, mm² | 0.765 | 0.782 | 0.377 | 0.737 | 0.771 | 0.028 |
| | (0.007) | (0.016) | | (0.006) | (0.010) | |
| Ec. Ar, mm² | 0.032 | 0.037 | 0.460 | 0.032 | 0.038 | 0.265 |
| | (0.003) | (0.006) | | (0.003) | (0.003) | |

S.V. n = 4,
S.G. n = 5,
O.V. n = 4,
O.G. n = 5;
Means (standard error) are presented;
p < 0.05

TABLE 5
Trabecular bone architectural properties in the distal femur metaphysis
was not significantly altered with GSK126 administration.

| | SHAM | | | OVX | | |
|---|---|---|---|---|---|---|
| | | | p- | | | p- |
| Property | Vehicle | GSK126 | value | Vehicle | GSK126 | value |
| BV/TV, % | 1.458 | 1.642 | 0.977 | 1.385 | 1.404 | 0.639 |
| | (0.195) | (0.295) | | (0.479) | (0.407) | |
| Tb.N, | 2.593 | 2.496 | 0.880 | 2.140 | 2.102 | 0.426 |
| mm⁻¹ | (0.064) | (0.088) | | (0.174) | (0.166) | |
| Tb.Th, | 0.040 | 0.042 | 0.191 | 0.041 | 0.047 | 0.638 |
| mm | (0.002) | (0.003) | | (0.003) | (0.002) | |
| Tb.Sp, | 0.385 | 0.402 | 0.740 | 0.479 | 0.499 | 0.338 |
| mm | (0.009) | (0.013) | | (0.035) | (0.045) | |
| SMI | 4.178 | 4.168 | 0.230 | 3.513 | 3.758 | 0.957 |
| | (0.066) | (0.140) | | (0.124) | (0.134) | |

S.V. n = 4,
S.G. n = 5,
O.V. n = 4,
O.G. n = 5;
Means (standard error) are presented

Taken together, the results provided herein demonstrate that while loss of EZH2 function creates abnormalities in skeletal patterning and bone formation in young animals, EZH2 inhibition in older and skeletally mature animals results in both bone anabolic and osteoprotective biological effects. These results demonstrate that EZH2 inhibition can be used to treat osteoporosis, enhance fracture healing, and promote new bone formation for the treatment of other medical conditions.

### Example 2—Anabolic and Anti-Resorptive Modulation of Bone Homeostasis by Sulforaphane, an Epigenetic Modulator and Isothiocyanate

### Cell Culture

The following murine cell lines were used: MC3T3-E1, a clonal pre-osteoblastic cell line derived from newborn mouse calvaria (obtained from Dr. Kumegawa, Department of Oral Anatomy, Meikai University, Sakado, Japan), the osteocyte-like MLO-Y4 cell line (obtained from Lynda Bonewald, University of Missouri-Kansas City, USA), and the pre-osteoclastic, macrophage-like RAW 264.7 cell line (ATCC, Manassas, Va., USA).

All cell lines were cultured in a humidified atmosphere with 5% CO₂ at 37° C. and were sub-cultured twice per week using 0.001% Pronase E (Roche Applied Science, Penzberg, Germany) and 0.02% EDTA in Ca²⁺- and Mg²⁺-free PBS before achieving confluence. MC3T3-E1 and MLO-Y4 cells were cultured in α-minimum essential medium (α-MEM; Biochrom, Berlin, Germany) containing 10 μg/mL gentamicin (Sigma-Aldrich, St. Louis, Mo., USA). For MC3T3-E1, culture media was supplemented with 10% heat inactivated fetal calf serum (FCS; Biochrom). MLO-Y4 cells were cultured on rat-tail derived collagen Type I (Roche) coated dishes (final concentration of 0.15 mg/mL). Culture media was supplemented with 2.5% FCS (Hyclone, GE Healthcare Life Sciences, Logan, Utah) and 2.5% calf serum (CS, Hyclone).

Differentiation of MC3T3-E1 cells was induced with 50 μg/mL ascorbic acid (Sigma-Aldrich) and 5 mM β-glycerophosphate (Sigma-Aldrich) in medium containing 5% FBS. RAW 264.7 cells were cultured in Dulbecco's modified Eagle's medium (DMEM, Invitrogen, Carlsbad, Calif., USA) supplemented with 10% fetal bovine serum (Hyclone) and 2 mM glutamine (Gibco, Carlsbad, Calif., USA). Cells were seeded in culture dishes at a density of 20,000 cells/cm² and cultured overnight unless stated otherwise. Before cells were treated with compounds, the culture medium was changed.

### Primary Cells and Tissues

Primary mouse tissues or cells were obtained according to procedures that conform to the regulatory guidelines of the Institutional Animal Care and Use Committee of the Medical University of Vienna. Primary mouse osteoclasts were harvested from 8-week old C57BL/6 mice, which were sacrificed, and bone marrow cells were isolated from tibias and femurs under aseptic conditions and cultured in α-MEM (Biochrom) containing 10% FCS (Biochrom), 10 mg/mL gentamycin (Sigma-Aldrich) and macrophage colony stimulating factor (MCSF1, 30 ng/mL). After 24 hours, non-adherent cells were seeded onto sterile, 300 μm thick dentin slices (elephant ivory) at 700,000 cells/cm² in αMEM supplemented with 10% FBS, 2 mM 1-glutamine, 30 μg/mL gentamycin, 20 ng/mL M-CSF, and 2 ng/mL RANKL (R&D Systems, Minneapolis, Minn., USA). Culture medium was changed twice per week, and cells were cultured for 14 days.

Mouse bone marrow mesenchymal stem cells were isolated from aseptically dissected long bones of 6-week old C57BL/6 mice (Himberg, Austria). The marrow cavities were flushed with sterile medium using a 25-gauge needle, and the culture was established in α-MEM supplemented with 10% FCS and 10 mg/mL gentamycin. After 48 hours of culture at 5% CO₂ and 37° C., the non-adherent cells were removed by gentle rinsing with PBS. At confluence, cells were harvested and seeded at a density of 3,000/cm² for cell experiments. For osteogenic differentiation, cells were cultured in α-MEM supplemented with 10% FBS, 10 mg/mL gentamycin, 100 nM dexamethasone, 10 mM β-glycerophosphate, and 50 μg/mL ascorbic acid with or without L- or DL-SFN. Culture medium was renewed twice a week, and cells were cultured for the periods indicated in the result section.

Calvariae from 2 to 3-day-old and from 7-week old C57BL/6 mice were dissected aseptically. The calvarial bone explants were cultured in 48-well plates in α-MEM (Biochrom) containing 10% FCS (Biochrom), 50 μg/mL ascorbic acid (Sigma-Aldrich), 5 mM β-glycerophosphate (Sigma-Aldrich), and 10 μg/mL gentamicin (Sigma-Aldrich). The day after dissection, medium was changed, and a part of the explants was treated with 3 μM L- or DL-SFN for 12 days. Thereafter, one part of the calvariae were fixed for 1 hour in 4% para-formaldehyde (PFA), and mineralization was measured by Alizarin Red S stain (Sigma-Aldrich). For this purpose, Alizarin Red S dye was extracted using 10% cetylpyridinium chloride (CPC) in 10 mM sodiumphosphate (pH=7.0) for 45 minutes at room temperature. Alizarin Red S absorbance was measured at 562 nm in a multi-plate reader (Tecan, Maennedorf, Switzerland) and normalized to total protein amount measured by bicinchoninic acid assay (Thermo Fisher Scientific, Waltham, Mass., USA). From the other part of the calvariae, total RNA was extracted.

### Histomorphometric Measurements/Assessment of Osteoclastic Resorption

After culturing the primary mouse osteoclasts for 14 days on dentin slices in the presence or absence of 3 μM L- or DL-SFN, substrates were put into water, sonicated for 10 minutes to remove living cells and air-dried. Photographs were obtained by reflected light microscopy (objective 20×) of the entire substrate surface, and resorption trails and pits were analyzed and quantified with standard image analysis software (ImageJ, rsbweb.nih.gov/ij/). Resorption was shown as % area resorbed to total area of the dentin slice.

### Cell Metabolic Activity

To assess cell metabolic activity, a commercially available, MTT similar assay (EZ4U; Biomedica, Vienna, Austria) was used. For this purpose, the cell lines were incubated with increasing concentrations of L- or DL-SFN. After a comparable doubling time for all three cell lines, the assay was performed following the protocol of the supplier.

### Cell Count

Cell lines were seeded in 24-well culture dishes at a density of 20,000/cm² and were either left untreated (controls) or treated with L- or DL-SFN at the indicated concentrations for up to 48 hours. Thereafter, cells were detached with 0.001% pronase E, and the number of viable cells was assessed with Casy cell counter (Schaerfe Systems, Reutlingen, Germany). Each experiment was performed in quadruplicate, and experiments were carried out twice. For long term experiments, cell number was determined using DNA amount as surrogate. Cell layers were washed with PBS and fixed for 20 minutes with 4% PFA. Thereafter, Hoechst 33258 dye (Polysciences, Warrington, Pa., USA) was added (1 μg/mL), and, after an incubation of 15 minutes at room temperature, the fluorescence was measured (excitation 360, emission 465 nm). The amount of DNA was estimated using a standard curve prepared from calf thymus DNA (Roche).

### Measurement of Caspase Activity

Caspase 3/7 and caspase 8 activities were measured by using the CASPASE-GLO® 3/7 and CASPASE-GLO® 8 assay Kit (Promega, Madison, Wis., USA) following manufactures instructions. Briefly, after treatments, cells were lysed, and substrate cleavage by caspases was measured by the generated luminescent signal with a 96 multi-well luminometer (Glomax, Promega). Each experiment was performed in quintuplicate and experiments were carried out twice.

Tet1 and Tet2 siRNA Transfections

For Tet1 and Tet2 depletion by siRNA, cells were seeded at 20,000 cells/cm² in six-well culture plates. Six hours after seeding, cells were transfected with 40 pmol of Tet1 or Tet2 siRNA (Sigma-Aldrich) using X-TREMEGENE™ siRNA Transfection Reagent (Roche) as described by the supplier. One day after transfection, medium change was performed, and one part of the cells was treated with medium containing 3 μM DL-SFN, while the other part was left untreated. After incubation time of 24 hours, nucleic acids were isolated as described herein and subjected to qRT-PCR or cell count was performed.

### Isolation of RNA and Reverse-Transcriptase Quantitative Polymerase Chain Reaction (RT-qPCR)

Total RNA was extracted using the SV Total RNA Isolation kit (Promega) following the supplier's instructions. cDNA was synthesized from about 0.5 μg RNA using the First Strand cDNA Synthesis kit (Roche) as described by the supplier. The resulting cDNAs were subjected to quantitative PCR amplification with a real-time cycler using the QUANTITECT™ SYBR® Green PCR Kit (Qiagen, Hilden, Germany) for the genes Alpl, Fas, Lox, Tet1, Tet2, and TAQMAN® Gene expression Master Mix (Applied Biosystems, Foster City, Calif., USA) for measuring Runx2, Bglap2, Colla1, Tnfsf11, and 18S rRNA expression (for primers, see Table 1). SYBR-Green RT-qPCR was started with an initial denaturation step at 95° C. for 10 minutes and then continued with 45 cycles consisting of 30 seconds denaturation at 95° C., 30 seconds annealing at primer-specific temperatures, and extension at 72° C. For measurement of the TAQMAN® assays, an initial denaturation at 95° C. for 10 minutes was applied, followed by 45 cycles alternating 60 seconds at 60° C. and 15 seconds at 95° C. (primers or TAQMAN® probe references are listed in Table 2). All RT-qPCR assays were performed in triplicate, and expression was evaluated using the comparative quantification method (Pfaffl, Nucleic Acids Res., 29:e45 (2001)).

### Protein Isolation and Immunoblotting

Whole cell protein extracts were prepared using SDS sample buffer (2% SDS, 100 mM β-mercaptoethanol and 125 mM Tris-HCl, pH=6.8) and heated at 95° C. for 5 minutes. To obtain supernatant protein extracts, cell medium was collected, and proteins were precipitated with 50% trichloroacetic acid at 4° C. for 1 hour, concentrated by centrifugation, neutralized with PBS, and dissolved in SDS sample buffer.

For immunoblotting analysis, 15 μg of protein extracts were separated on 10% SDS poly-acryl amide gels, transferred to nitrocellulose membranes (Millipore), and blocked overnight with 10% blocking reagent (Roche) in TN buffer (50 mM Tris and 125 mM NaCl, pH=8.0). The following primary antibodies were used: rabbit, anti-Runx2 (sc-10758, Santa Cruz Biotechnologies, Santa Cruz, Calif.) and rabbit, anti-β-Actin (#4967, Cell Signaling, Danvers, Mass., USA). Washing was performed with TN buffer containing 0.01% Tween. Binding of the secondary antibody (anti-rabbit IgG/anti-mouse IgG horseradish peroxidase-coupled) (Roche) diluted 1:10,000 in 10% blocking solution followed by detection with the BM chemo-luminescence immunoblotting kit (Roche) was carried out as described by the supplier. Chemo-luminescence was measured with an image acquisition system (Vilber Lourmat, Marne-la-Vallée, France). Measurements are given as means of 3 immuno-blots, and representative blots are shown.

### Immunostaining and Quantifications of Global Cytosine 5-Hydroxymethylation

Cells were fixed with 4% paraformaldehyde in PBS for 20 minutes at room temperature and permeabilized with 0.2% triton in 4% paraformaldehyde in PBS. Thereafter, cell layers were blocked for 20 minutes with 10% blocking reagent (Roche) and incubated for one further hour with 0.5 μg/mL anti-5hmC antibody (Abgent). Afterwards, the cells were washed thrice with PBS and incubated for one further hour with an Alexa488 labeled secondary antibody (Invitrogen) diluted 1:300 in blocking buffer. Finally, nuclei were stained with Hoechst 33258 dye (Polysciences).

For immunostaining, cell slides were mounted with VECTASHIELD® mounting media, and immunofluorescence was visualized on a Leica laser-scanning microscope. No signal was found by using only the Alexa488 labeled secondary antibody (Invitrogen). For quantification of global cytosine 5hmC, the fluorescence in the plates was measured with a multiwell plate reader (Tecan). The amount of DNA (nuclei signal) was estimated using a standard curve prepared from calf thymus DNA (Roche). The signal of the fluorescence of the 5hmC staining was normalized to the amount of DNA. Also, in this case, no signals were found when only the Alexa488 labeled secondary antibody (Invitrogen) was used.

### In Vivo Analysis of SFN Effects on Bone

Analysis of SFN on mice was performed using a protocol similar to that described elsewhere (Kong et al., Arthritis Rheum., 62:159-170 (2010)). In the experimental setting herein, eight week old sham operated or ovariectomized (OVX) C57B16/J female mice were purchased from Charles River laboratories. Mice were injected with 7.5 mM DL-SFN (200 μL of SFN at a concentration of 63.8 mg/mL/kg dissolved in ethanol and further diluted in PBS) intraperitoneally every other day for five weeks. Control mice received vehicle alone. As described elsewhere (Kong et al., Arthritis. Rheum., 62:159-170 (2010)), the injection of this dose of DL-SFN did not show apparent adverse effects, including weight loss, alterations in physical appearance, or changes in behavior in the treated mice.

### Micro Computed Tomography (μCT) Analysis

Images from tibias fixed in 4% formaldehyde were acquired in a SkyScan 1174 with a resolution of 6 μm (X-ray voltage 50 kV). Image reconstruction was performed by applying a modified Feldkamp algorithm using the Skyscan Nrecon software. 3D and 2D morphometric parameters were calculated for the trabecular bone (350 consecutive slides, 6 μm thick) starting from 300 μm from the growth plate. Threshold values were applied for segmenting trabecular bone corresponding to bone mineral density values of 0.6/cm³ calcium hydroxyapatite. 3D parameters were based on analysis of a Marching Cubes type model with a rendered surface. Calculation all of 2D areas and perimeters was based on the Pratt algorithm (Pratt et al., IEEE Trans. Biomed. Eng., 38, 306-309 (1991). Bone structural variables and nomenclature were used according to Bouxsein et al. (J. Bone Miner. Res., 25:1468-1486 (2010)).

Quantitative Backscattered Electron Imaging (qBEI)

Un-decalcified distal femora from 13 weeks old mice, sham/Crt (n=5), sham/DL-SFN (n=5), OVX/ctrl (n=4) and OVX/DL-SFN (n=5), were fixed and dehydrated in alcohol, embedded in polymethylmethacrylate (PMMA), and prepared for backscattered electron imaging as previously described. qBEI is based on backscattering of electrons from the surface-layer (i.e., the initial ˜1.5 micron) of a bone section. The rate of these backscattered electrons is proportional to the weight concentration of mineral (hydroxyapatite), and thus of that of calcium in bone. Details of the method were described elsewhere (Roschger et al., Bone, 42:456-466 (2008)). A scanning electron microscope (DSM 962, Zeiss, Oberkochen, Germany) equipped with a four quadrant semiconductor backscattered electron detector was used. The accelerating voltage of the electron beam was adjusted to 20 kV, the probe current was adjusted to 110 pA, and the working distance was adjusted to 15 mm. The cancellous and cortical bone areas were imaged at 200× nominal magnification (corresponding to a pixel resolution of 1 μm/pixel). The BE-signal (gray scale) was calibrated using the “atomic number contrast” between carbon (C, Z=6) and aluminum (Al, Z=13) as reference materials. From the calibrated digital images frequency distribution of mineral concentration, the so called bone mineralization density distribution (BMDD) was derived. The BMDD was characterized by five parameters: CaMean (weighted mean Calcium content), CaPeak (mode Ca content—peak position), CaWidth (full width at half maximum of the BMDD peak—reflecting the heterogeneity in matrix mineralization), CaLow (the percentage of lowly mineralized bone area—below 17.68 weight % Ca), and CaHigh (the percentage of highly mineralized bone area—above the 95th percentile value of the corresponding control animals BMDD).

### Statistical Analysis

Statistical analyses were performed using ANOVA or Student's t test in Prism 4.03 (GraphPad Software, La Jolla, Calif., USA). Values of P≤0.05 were considered significant. Each experiment consisted of at least three biological replicates. For RT-qPCR data, results from technical triplicates were averaged, and the mean value was treated as a single, biological statistical unit. Results were presented as means±SD.

### Results

### SFN Affects Cell Viability of Bone Related Cells

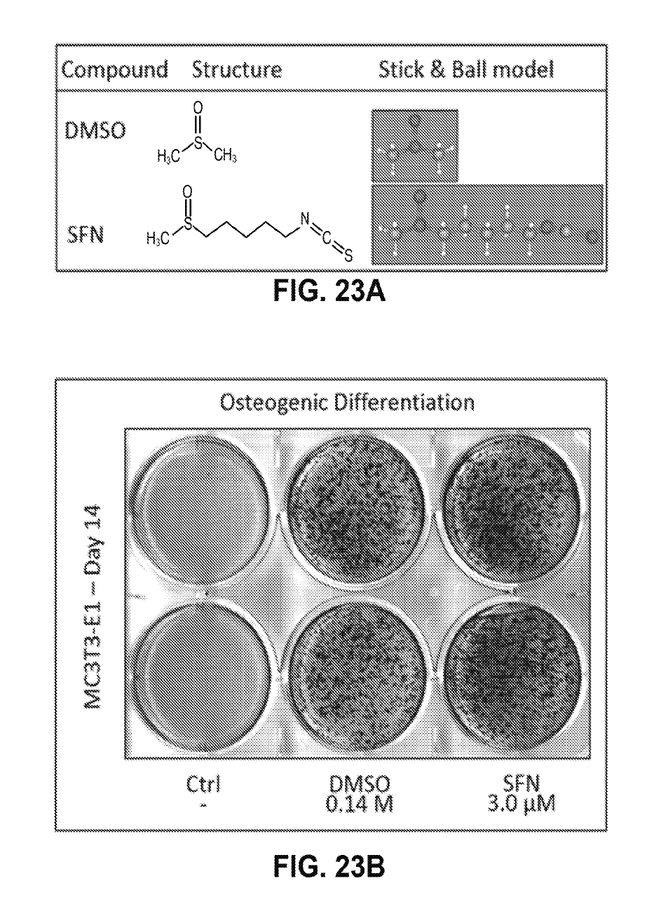

Cryopreservant dimethylsulfoxide (DMSO) triggers differentiation of MC3T3-E1 osteoblasts. Although use of DMSO was restricted in medical applications, there are intriguing analogies in the chemical structures of DMSO and plant secondary metabolites. As deduced from the IUPAC nomenclature, DMSO (methanesulfinyl methane) and the natural food compound SFN (1-isothiocyanato-5-methanesulfinylpentane) share striking molecular similarities. SFN carries an additional pentane group with terminal isothiocyanate (FIG. 23A). These structural similarities predict similarities in the biological effects of DMSO and SFN.

The effects of DMSO and SFN on MC3T3-E1 differentiation was examined by assessing the extent of Extra Cellular Matrix (ECM) mineralization. Under the tested conditions, staining for the late osteoblast biomarker Alizarin Red was not observed in the cultures for the first two weeks of culture, but typically was evident after three to four weeks (Fratzl-Zelman et al., Bone, 23:511-520 (1998)). The results showed that both DMSO and SFN have similar biological effects as revealed by increased matrix mineralization as early as 14 days of treatment (FIG. 23B). Strikingly, SFN was at least four orders of magnitude more potent than DMSO in stimulating matrix mineralization, suggesting that the methanesulfinyl group of DMSO was important, but not sufficient for full biological activity.

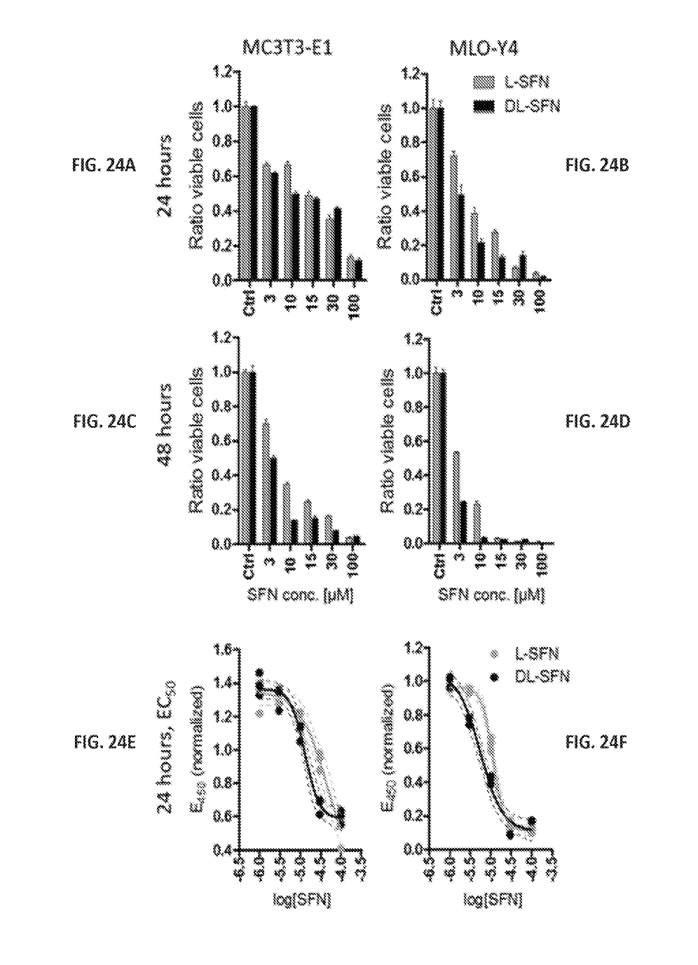

SFN exhibited cell growth suppressive effects (Gamet-Payrastre et al., Cancer Res., 60:1426-1433 (2000); Singh et al., J. Biol. Chem., 279:25813-25822 (2004); and Gamet-Payrastre, Curr. Cancer Drug Targets, 6:135-145 (2006)). The impact of SFN on cell proliferation and viability of bone related immortalized cell-lines was evaluated. MC3T3-E1 osteoblasts and the MLO-Y4 osteocytes were treated with increasing concentrations of SFN for up to 48 hours (FIG. 24). Because SFN is a chiral molecule, two different preparations were tested: L-SFN and DL-SFN, which is a mixture of both D and L enantiomers. Both L-SFN and DL-SFN exhibited modest effects (<50%) on cell number in MC3T3-E1 and in MLO-Y4 after 24 hours of treatment at a concentration of 3 μM (FIGS. 24A and 24B). At higher concentrations, both the L-enantiomer and the DL-mixture compromise cell viability with the greatest decrease in cell number observed for MLO-Y4 cells (FIG. 24B). Furthermore, DL-SFN exhibited a somewhat stronger effect on cell survival compared to L-SFN in MLO-Y4 cells at most concentrations. Effects of L-SFN or DL-SFN on cell viability were slightly more pronounced after 48 hour treatment in both MC3T3-E1 and MLO-Y4 cell lines (FIGS. 24C and 24D). The preparation with both enantiomers (L- and DL-SFN) showed stronger biological effects compared to the sole L-enantiomer, suggesting that the biological potency of SFN depended on steric considerations. Taken together, the results demonstrate that 3 μM SFN is a relatively non-toxic dose (FIG. 24) that is biologically effective (FIG. 23).

To assess whether SFN affects cellular metabolic activity after 24 hours of treatment, EZ4U assays, an MTT-like assay that measures the capability of living cells to reduce tetrazolium salts in the mitochondria into formazan derivatives that absorb at 450 nm, were performed. Titration curves for both L-SFN and DL-SFN using either cell line revealed that the half-maximal effective concentration (EC₅₀) of L-SFN was about 48 μM and DL-SFN was about 13 μM in MC3T3-E1 cells (FIG. 24E), while lower doses were needed in MLO-Y4 cells (i.e., about 11 μM for L-SFN and about 6 μM for DL-SFN) (FIG. 24F). Hence, SFN exhibited less metabolic effects on MC3T3-E1 osteoblasts, than in MLO-Y4 osteocytes.

### SFN Induces Extrinsic Apoptosis in Cells of the Osteoblastic Lineage

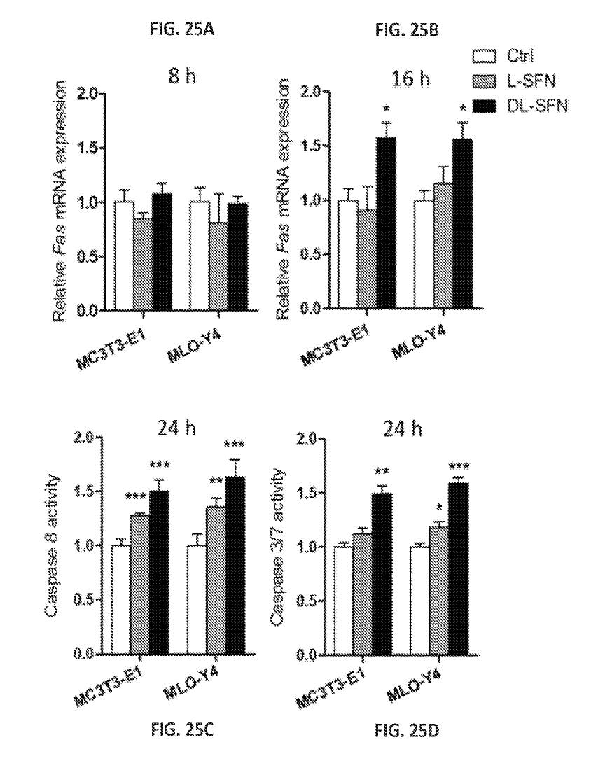

DMSO induced the extrinsic pathway of apoptosis (Thaler et al., Epigenetics, 7:635-651 (2012)). The growth suppressive action of SFN (as reflected by a decrease in cell proliferation and metabolic activity; see FIG. 23) may be caused by programmed cell death in both, MC3T3-E1 osteoblasts and MLO-Y4 osteocytes. To test this possibility, expression levels and activity of apoptotic biomarkers were monitored. Fas mRNA expression was not affected by either L- or DL-SFN in MC3T3-E1 and MLO-Y4 cells after 8 hours of treatment (FIG. 25A). However, after 16 hours of treatment, Fas mRNA expression was significantly increased in both cell types by DL-SFN treatment (FIG. 25B). Corroborating these results, the activity of caspase 8 was significantly increased by L-SFN and DL-SFN after 24 hours (FIG. 25C). Furthermore, a significant increase was observed in the activities of the caspases 3/7 upon DL-SFN treatment of MC3T3-E1 cells and MLO-Y4 cells for 24 hours (FIG. 25D). These results indicate that SFN induces the extrinsic apoptotic pathway in osteogenic cells.

### Osteogenic Long-Term Effects of SFN

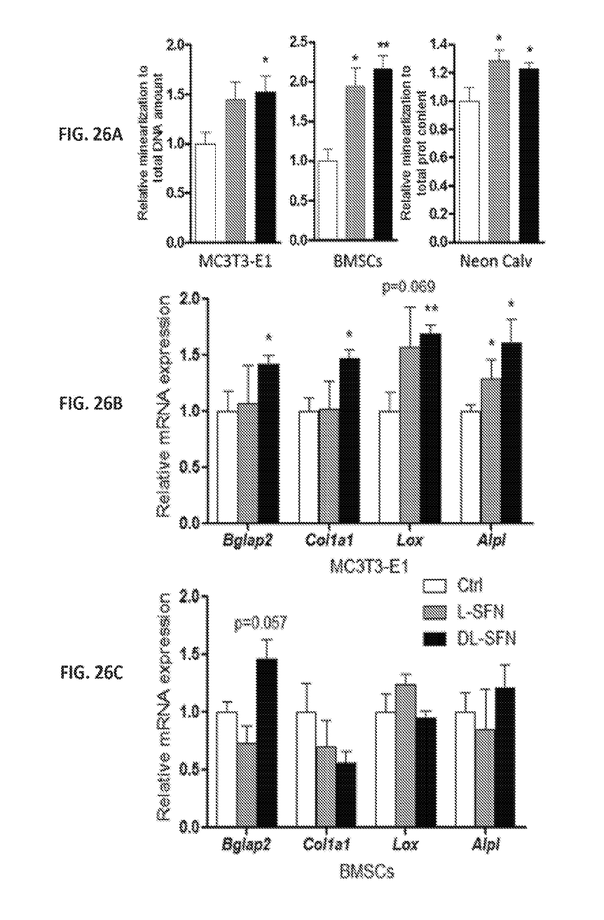

Beyond effects on cell growth and survival, SNF may affect osteoblast differentiation and activity. Therefore, the long-term effects (>20 days) of L-SFN and DL-SFN on mineralization of osteoblast, bone marrow stromal/stem cell (BMSC) cultures, and neonatal calvarial explants maintained in osteogenic media were investigated. Treatment of MC3T3-E1 cells or BMSCs (FIG. 26A) significantly increased ECM mineralization, the principal marker for mature osteoblast function. Because L-SFN and DL-SFN exhibited mild effects on cell survival (at 3 μM) in non-confluent MC3T3-E1 osteoblasts (FIGS. 24A and 24C), cell number of both MC3T3-E1 cells and BMSCs in response to both enantiomer preparations were assessed. MC3T3-E1 and BMSCs cultures treated with either SFN type exhibited modest lower cell numbers, but higher mineral content per DNA unit after long-term treatment, indicating that SFN treated cells were more active in mineralization. To evaluate if this bone anabolic effect occurs in explants, which are rich in post-proliferative mature osteoblasts, ECM mineralization of newborn calvarial explants treated with 3 μM L- or DL-SFN for 14 days was measured. As in the 2D cell culture models, L- and DL-SFN each significantly increased ECM mineralization in calvarial explants (FIG. 26A). These results indicate that SFN is capable of biologically stimulating mineralization of neonatal bone tissue in culture. Next, SFN was assessed to see if it selectively modulates mRNA expression of differentiation related genes that support mineralization. Treatment of mouse MC3T3-E1 cells with 3 μM of L or DL-SFN for 14 days increased the mRNA expression of prominent osteoblastic markers including the ECM related genes Bglap2, Colla1, Lox and Alpl (FIG. 26B). In BMSCs, up-regulation of Bglap2 mRNA expression was observe after the same treatment time (FIG. 26C). DL-SFN generally exhibited a more pronounced effect than L-SFN indicating that the osteogenic activity of SFN may have a stereo-chemical component. Taken together, these results indicate that SFN promotes the expression of osteoblastic genes and mineralization.

### The Bone-Anabolic Effects of SFN are Reflected by Enhanced Expression of Runx2

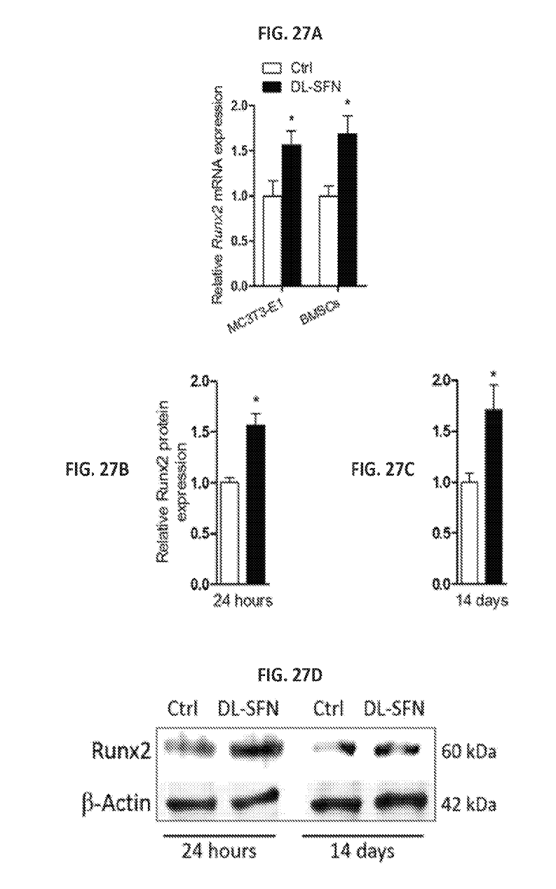

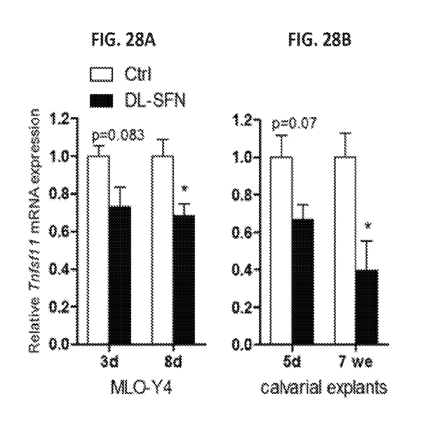

SFN may play a role in cellular differentiation by selectively modulating mRNA expression of osteogenic transcription factors. Therefore, SFN was examined to see if it affects the mRNA and protein expression of the bone-related master regulator Runx2 in mouse MC3T3-E1 cells or BMSCs. Treatment of cells with DL-SFN stimulates Runx2 mRNA expression in both cell types (FIG. 27A). Immunoblot analysis of DL-SFN-treated MC3T3-E1 cells revealed a significant increase in Runx2 protein expression after both 24 hours (FIG. 27B) and 14 days (FIG. 27C) of treatment, thus confirming the pro-osteogenic effects of SFN (FIG. 26). Representative immune blots of Runx2 protein expression are shown in FIG. 27D. The increase in Runx2 protein at 24 hours indicated that SFN may already have mechanistic effects at relatively early stages of osteoblastic differentiation.

### SFN Attenuates RANKL/Tnfsf11 Expression in Mouse Calvarial Explants

Runx2 expressed in osteoblastic cells mediates biological feed-back by promoting osteoclast differentiation via up-regulation of Tnfsf11 (RANKL) (Enomoto et al., J. Biol. Chem., 278:23971-23977 (2003)). The latter promoted osteoclast formation and survival by binding to the osteoclastic receptor Tnfrsf11a (alias RANK), which in turn activates NFkB (Shiotani et al., Anat. Rec., 268:137-146 (2002)). Furthermore, osteocyte-derived Tnfsf11 controls bone remodeling during postnatal development and in adult mammals. In osteocyte-like MLO-Y4 cells, DL-SFN reduced Tnfsf11 mRNA expression at both 3 and 8 days after treatment, although changes in expression were only significant at the latter time-point (FIG. 28A). In addition, SFN decreased Tnfsf11 expression in mouse calvarial explants from neo-natal mice and to a greater extent in explants from adult mice after 12 days of treatment with DL-SFN ex vivo (FIG. 28B). These results indicate that SFN may potentially promote net bone accumulation by suppressing RANKL/Tnfsf11 induced osteoclast formation.

### SFN Induces Global DNA Hydroxymethylation Changes

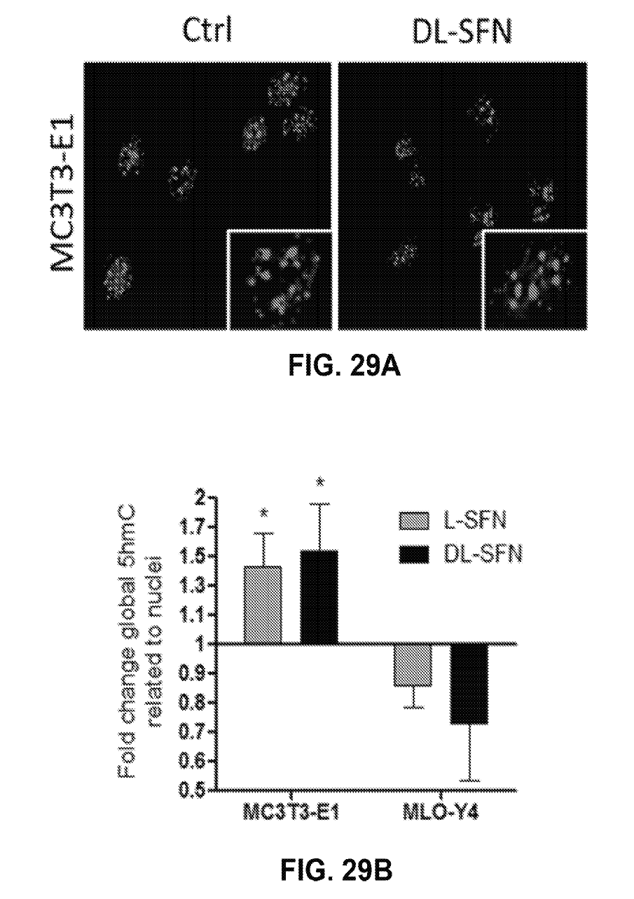

DMSO dramatically induced active DNA demethylation in pre-osteoblastic MC3T3-E1 cells within less than one day of treatment (<16 hours) through formation of 5-hydroxy-methylcytosine (5hmC) (Thaler et al., Epigenetics, 7:635-651 (2012)). Due to the structural and biological similarities between DMSO and SFN, SFN was analyzed to see if it alters the level of active DNA demethylation in cells of the osteogenic lineage. Indeed, a significant global increase in 5hmC was measured as marker for ongoing active DNA demethylation upon treatment with either L- or DL-SFN in MC3T3-E1 cells at 16 hours after treatment based on immunofluorescence laser-scanning microscopy (FIG. 29A) and spectrophotometry (FIG. 29B). Interestingly, in MLO-Y4 osteocytes that are differentiated beyond the osteoblastic stage, both L- and DL-SFN did not elevate 5hmC levels (FIG. 29A). These findings collectively indicate that SFN selectively induces active DNA demethylation in osteoblastic cells that are at rather early than at late stages of osteogenic differentiation.

### SFN Transiently Enhances Expression of Tet1 in Osteoblastic Cells

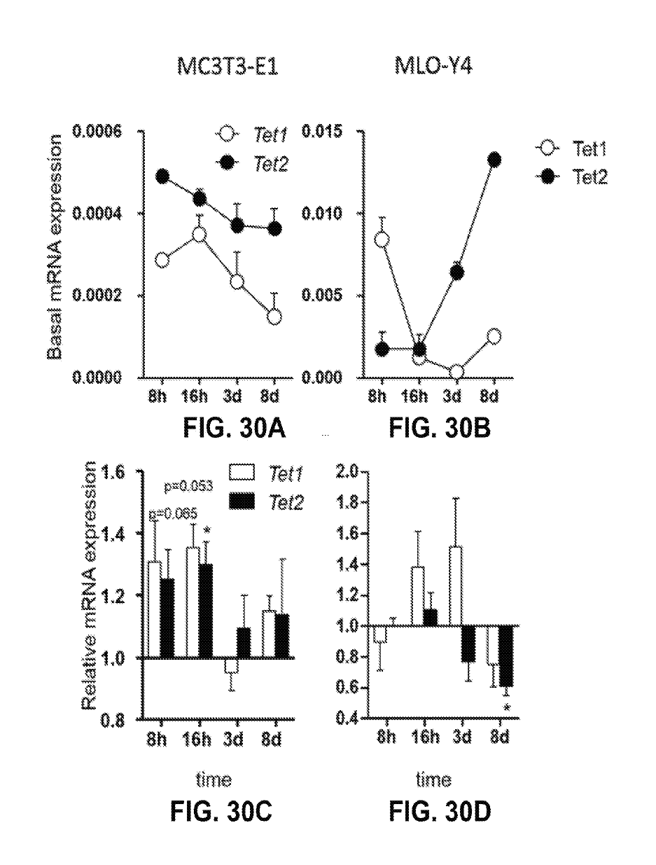

Global changes in 5hmC levels are mediated by enzymes that control DNA-hydroxymethylation and are encoded by Ten-eleven translocation (Tet) genes. Tet1 and Tet2 were each expressed in MC3T3-E1 cells, and expression for each gene generally decreased within the first 8 days of MC3T3-E1 osteoblast differentiation (FIG. 30A). Yet, the levels of Tet1 and Tet2 were distinctly regulated during early stages in MLO-Y4 cells. Tet1 was much more rapidly down-regulated in MLO-Y4 cells compared to MC3T3-E1, while Tet2 levels were steadily up-regulated (FIG. 30B). mRNA expression of Tet1 and Tet2 genes were examined in the absence or presence of SFN in both MC3T3-E1 and MLO-Y4 cells. Beyond the modulations in Tet1 and Tet2 levels in response to biological induction of differentiation, expression of Tea and Tet2 increased to a limited degree (between 20 and 40%) by DL-SFN at 8 hours and 16 hours after treatment in MC3T3-E1 cells. These values reached statistical significance only for Tet1 at the 16 hour time point (FIG. 30C). This modest stimulation of Tet1 and Tet2 expression by SFN results in a significant increase in global 5hmC formation in MC3T3-E1 cells (FIG. 29). DL-SFN did not positively change mRNA expression for Tet1 or Tet2 in MLO-Y4 cells (FIG. 30D). mRNA levels of the hydroxylation enzymes Tet1 and Tet2 were modestly regulated by SFN during bone cell differentiation, and may perhaps contribute to DL-SFN induced changes in global hydroxymethylation.

### Effects of SFN on Osteoclast Resorption and Apoptosis

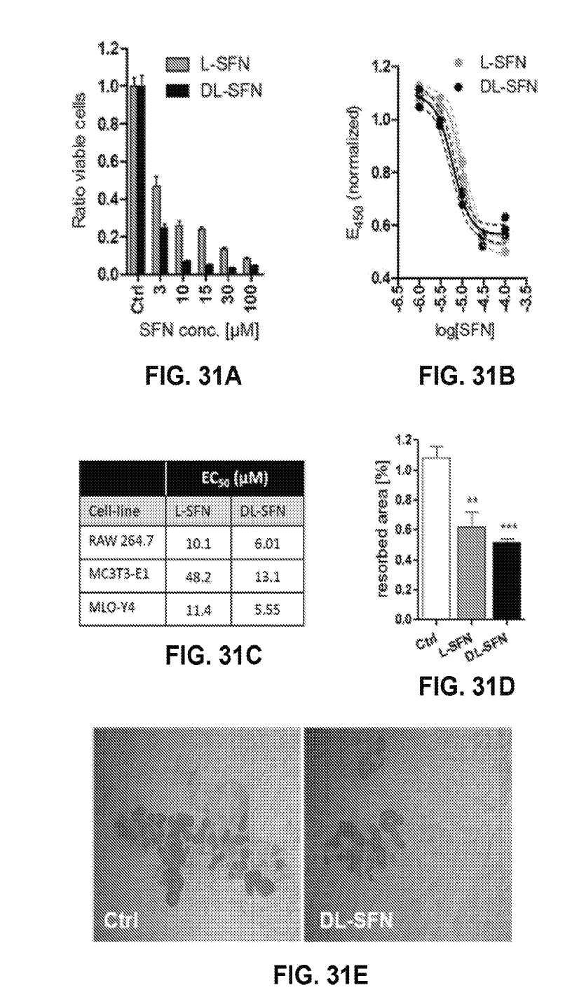

Because SFN inhibits osteoclastogenesis by suppressing nuclear factor-kappaB (NFkB) (Kim et al., Mol. Cells, 20:364-370 (2005)) and it also has an anti-proliferative effect in bone-anabolic MC3T3-E1 and MLO-Y4 cells, SFN was examined to see if it affects proliferation and activity of RAW 264.7 cells. Treatment of these cells with increasing concentrations of SFN for 24 hours with the two different preparations of SFN (i.e., L-SFN and DL-SFN) reduced the number of viable cells at doses of 3 μM and higher (FIG. 31A). This inhibitory effect appeared more pronounced compared to observations made with osteogenic cell lines (FIG. 24). Furthermore, SFN strongly reduced the cellular metabolic activity of RAW 264.7 cells in EZ4U assays (FIG. 31B). Also, for this parameter, a stronger effect in the RAW 264.7 cells was observed when compared with the osteogenic cell-lines (FIG. 31C). These negative effects were assessed to see if they altered the functional capacity of osteoclasts in bone resorption. Treatment with SFN significantly reduced the area resorbed by primary mouse osteoclast cultures on ivory discs (FIGS. 31D and 31E). SFN also was investigated to see if it alters progression of osteoclastic differentiation in RAW 264.7 cells upon induction with RANKL and MCSF by monitoring the temporal expression of classical osteoclastic markers (e.g., Acp5, Clcr, Ctsk). However, no appreciable changes in the expression of these genes upon co-treatment with SFN was observed. Hence, SFN primarily affects activity of osteoclastic cells via its cytostatic effects.

### SFN Induces FAS-Dependent Extrinsic Apoptosis in Pre-Osteoclastic Cells

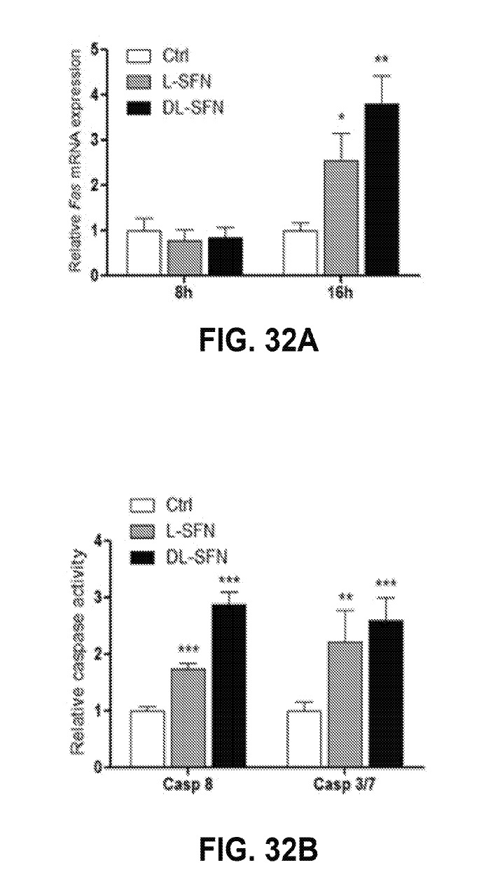

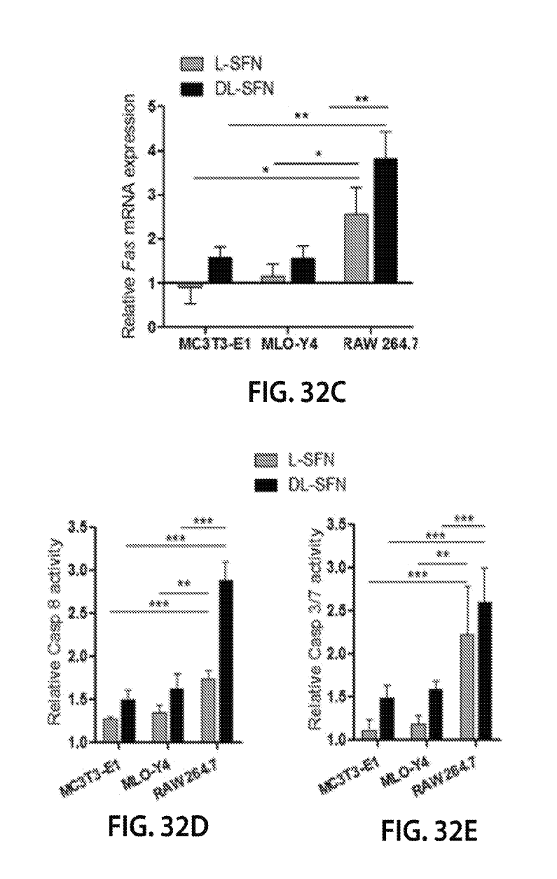

Because SFN induces apoptotic markers in MC3T3-E1 osteoblasts and MLO-Y4 osteocytes (FIG. 25), RAW 264.7 cells were examined to determine if they are sensitive to programmed cell death. Similar to MC3T3-E1 and MLO-Y4 cells, the results revealed that treatment of RAW 264.7 pre-osteoclastic cells with 3 μM of L- or DL-SFN up-regulated expression of the pro-apoptotic gene Fas at 16 hours but not at 8 hours (FIG. 32A). In addition, either SFN preparation stimulated Caspase 8 and Caspase 3/7 activity (FIG. 32B), indicating that SFN induces the Fas-Caspase8-Caspase3/7 pathway that defines extrinsic apoptosis. Similar to findings with MC3T3-E1 osteoblasts and MLO-Y4 osteocytes (FIG. 25), the DL-SFN mixture was more potent than the pure L-SFN enantiomer in inducing apoptosis (FIGS. 32A and 32B). Bone-resorbing RAW 264.7 osteoclasts were twice as sensitive to apoptotic induction as the bone-anabolic MC3T3-E1 osteoblasts and MLO-Y4 osteocytes (FIGS. 32C-32E). These findings indicate that SFN may positively affect bone homeostasis in vivo by preferentially inducing apoptosis in osteoclasts.

### SFN Induces Global DNA Hydroxymethylation and Controls Tet1 Dependent Cell Death in Osteoclasts

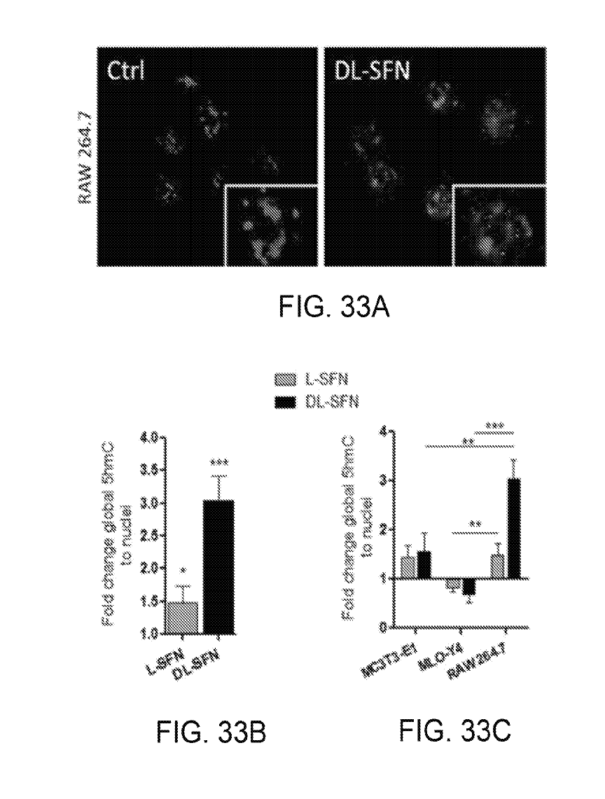

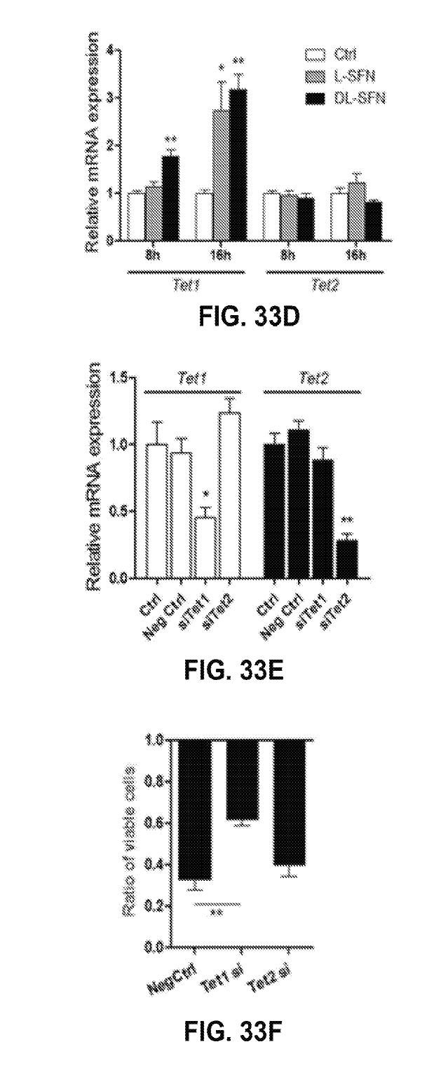

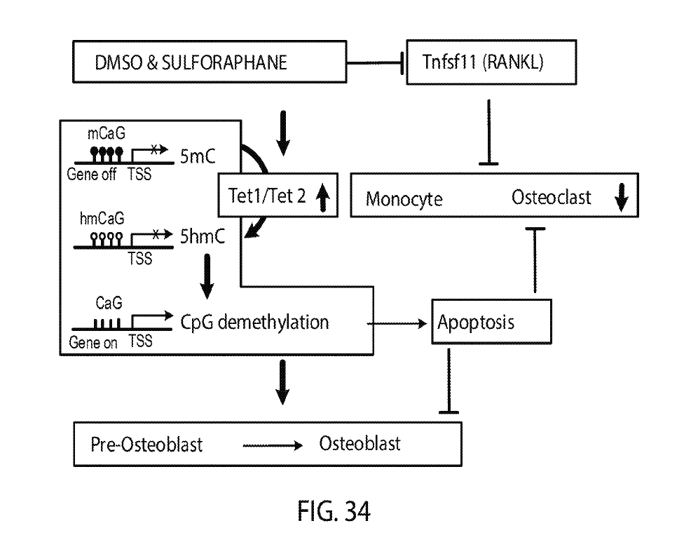

The following was performed to test if SFN alters active DNA demethylation through formation of 5hmC in RAW 264.7 pre-osteoclasts. Treatment of these cells with DL-SFN for 16 hours strongly increased the global levels of 5hmC (˜3 fold) as measured by immunofluorescence microscopy (FIG. 33A) and spectrophotometry (FIG. 33B). The L-SFN enantiomer that has lower biological activity exhibited a less pronounced effect (<2 fold) (FIG. 33B). The observed changes in 5hmC levels were most prominent in RAW 264.7 cells compared to MC3T3-E1 and MLO-Y4 cells (FIG. 33C). The mRNA levels of the hydroxymethylation-related gene Tet1, but not Tet2, were upregulated after DL-SFN treatment in RAW 264.7 cells (FIG. 33D). The effect of SFN on Tet1 mRNA levels was evident at 8 hours, but was more pronounced at 16 hours (FIG. 33D). These results were consistent with the idea that Tet-dependent hydroxymethylation may compromise cell survival as described elsewhere (Thaler et al., Epigenetics, 7:635-651 (2012)). To test this hypothesis, RAW 264.7 cells were treated with non-silencing RNA (negative control) or siRNAs for either Tet1 or Tet2 (FIG. 33E). Depletion of Tet1, but not Tet2, reduced the cytostatic effect of DL-SFN (FIG. 33F). This result suggests that Tet1 dependent hydroxymethylation supports FAS-mediated apoptosis. Taken together, these results demonstrate that the two organosulfur compounds DMSO and SFN induce active DNA demethylation via up-regulation of the Tet genes in vitro. This epigenetic reprogramming of the chromatin leads to apoptosis of pre-osteoclasts, but only to a lower extent of pre-osteoblasts. Active DNA demethylation leads to enhanced osteoblast differentiation without promoting osteoclast differentiation. Furthermore, in osteocytes and bone tissue explants, SFN decreased the expression of RANKL/Tnfsf11, a major activator of osteoclastogenesis. The mechanism of this modulation could be associated with reduced reactive oxygen species required for osteoclast differentiation (Gambari et al., Pharmacol. Res., 87:99-112 (2014)) (FIG. 34).

### SFN has Anabolic and Anti-Resorptive Effect on Bone Homeostasis In Vivo

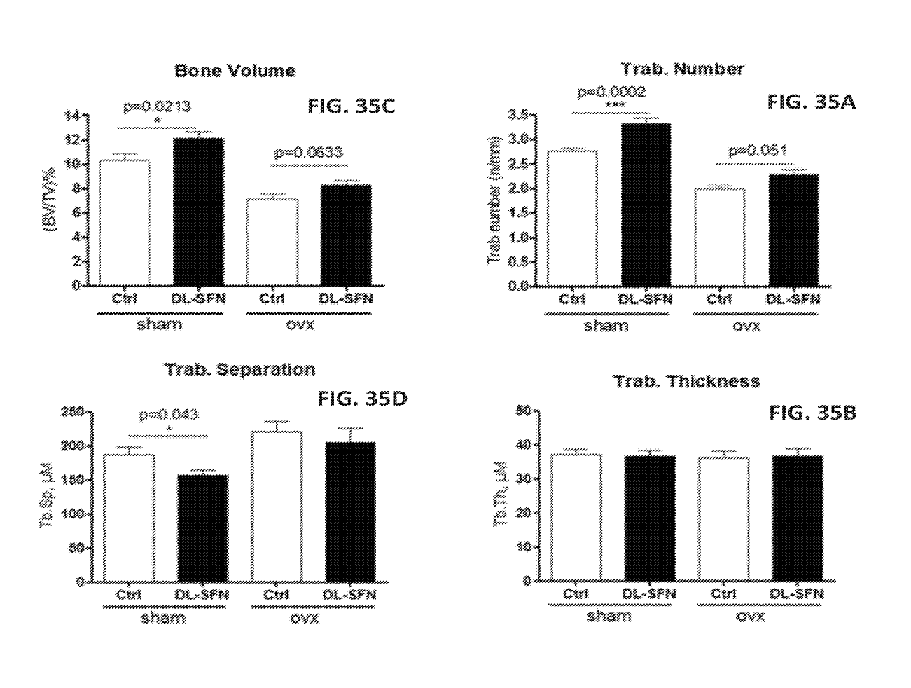

The in vitro results demonstrate that SFN stimulates osteogenic differentiation and acts as an anti-resorptive by blocking osteoclastogenesis and increasing osteoclast apoptosis. The biological effects of SFN were investigated in ovariectomized (OVX) mice that exhibit bone loss due to estrogen deficiency (FIG. 35). Treatment of sham-operated young adult mice (8 week old) with DL-SFN for five weeks revealed significantly higher femoral metaphyseal cancellous bone volume per tissue volume (BV/TV) (FIG. 35A) and trabecular number (Tb.N) (FIG. 35B), while decreasing trabecular separation (Tb.Sp.) (FIG. 35C) but not affecting trabecular thickness (Tb.Th.) (FIG. 35D). Thus, the relative higher BV/TV values found in the DL-SFN treated animal groups were reflected by changes in trabecular number and not due to a thickening of the trabeculae by enhanced bone apposition. SFN treatment of OVX mice reduced bone loss due to estrogen-deficiency as reflected by mitigation of the observed OVX-dependent decrease of trabecular number (FIGS. 35A and 35B).

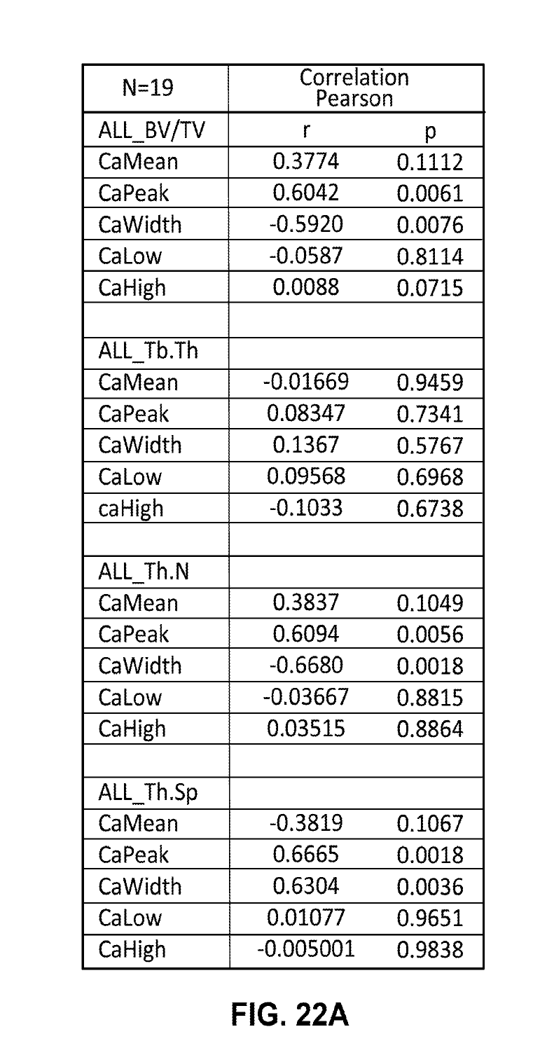

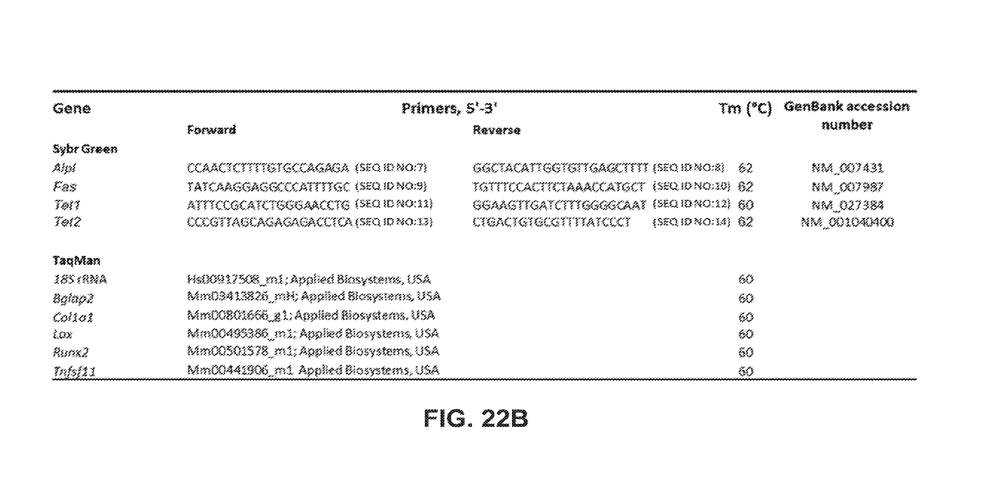

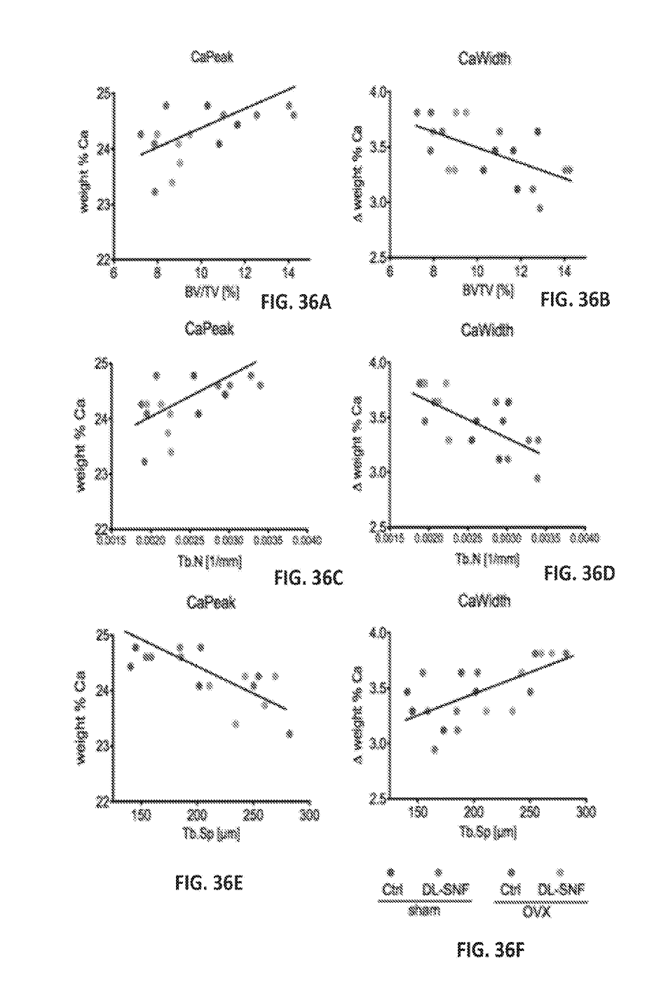

To complement the μCT results on bone micro-structural indices, the mineralization status of the bone matrix was measured by assessing local mineral content and distribution using quantitative back-scattered electron imaging (qBEI). Mice were examined for changes in bone mineral density distribution (BMDD) parameters CaMean, CaPeak, CaWidth, CaLow, and CaHigh reflecting bone turnover, mineralization kinetics, and average bone matrix age (Roschger et al., Bone, 23:319-326 (1998); and Fratzl-Zelman et al., Bone, 44:1043-1048 (2009)) (FIGS. 22A and 36). Two-way ANOVA analysis of CaPeak and CaWidth of trabecular bone revealed significant differences between control (sham) and estrogen-depleted (OVX) mice, but no effects for treatment with DL-SFN. The parameters CaPeak and CaWidth correlated significantly with the corresponding μCT parameters BV/TV, Tb.N and Tb.Sp (FIGS. 22A and 36A-36F). Independently of the treatment, it appeared that mice with a higher trabecular number have higher matrix mineralization (CaPeak, FIG. 36C), which was less heterogeneous (CaWidth, FIG. 36D), while mice with a low trabecular number had a less mineralized matrix, which tended to be more heterogeneous. Notably, the correlations between structural indices and BMDD parameters revealed a clear separation between the sham and OVX group. Consistent with the expected lower bone turnover rates in sham versus OVX treated mice, sham exhibited higher CaPeak and lower CaWidth values than the OVX group. Taken together, these results demonstrate that DL-SFN can have a beneficial effect on bone volume by mitigating loss in trabecular bone number due to both anabolic and anti-resorptive cellular effects without significantly changing bone matrix mineralization.

Taken together, these results demonstrate that a food-derived compound SFN epigenetically stimulates osteoblast activity and diminishes osteoclast bone resorption, shifting the balance of bone homeostasis in favor of bone acquisition and/or mitigation of bone resorption in vivo. Thus, SFN is a member of a new class of epigenetic compounds that can be used to counteract osteoporosis.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for reversing osteoporosis in a mammal, wherein the method comprises:
(a) identifying a mammal as having osteoporosis, and
(b) administering an inhibitor of an EZH2 polypeptide to the mammal, thereby reversing the osteoporosis.

2. The method of claim 1, wherein the mammal is a human.

3. The method of claim 1, wherein the inhibitor is selected from the group consisting of GSK126, UNC1999, EPZ005687, GSK343, EPZ-6438, and EI1.

4. A method for preventing the onset of osteoporosis in a mammal at risk for developing osteoporosis, wherein the method comprises:
(a) identifying a mammal as being at risk for developing osteoporosis, and
(b) administering an inhibitor of an EZH2 polypeptide to the mammal, thereby preventing the onset of osteoporosis.

5. The method of claim 4, wherein the mammal is a human.

6. The method of claim 4, wherein the inhibitor is selected from the group consisting of GSK126, UNC1999, EPZ005687, GSK343, EPZ-6438, and EI1.

7. A method for treating a bone injury or bone defect in a mammal, wherein the method comprises:
(a) identifying a mammal as having a bone injury or defect, and
(b) administering an inhibitor of an EZH2 polypeptide to the mammal, thereby enhancing bone healing and repair.

8. The method of claim 7, wherein the mammal is a human.

9. The method of claim 7, wherein the inhibitor is selected from the group consisting of GSK126, UNC1999, EPZ005687, GSK343, EPZ-6438, and EI1.

10. The method of claim 7, wherein the bone injury is a fracture.

11. A method for enhancing joint fusion in a mammal, wherein the method comprises:
(a) identifying a mammal as being in need of a procedure to promote joint fusion, or as having undergone a procedure to promote joint fusion, and
(b) administering an inhibitor of an EZH2 polypeptide to the mammal, thereby promoting bone formation and enhancing the rate and/or strength of joint fusion.

12. The method of claim 11, wherein the mammal is a human.

13. The method of claim 11, wherein said inhibitor is selected from the group consisting of GSK126, UNC1999, EPZ005687, GSK343, EPZ-6438, and EI1.

14. The method of claim 11, wherein the procedure is spinal fusion.

15. A method for enhancing implant ingrowth in a mammal, wherein the method comprises:
(a) identifying a mammal as being in need of, or as having, an implant that undergoes osteo-integration, and
(b) administering an inhibitor of an EZH2 polypeptide to the mammal, thereby enhancing the rate and/or strength of implant osteo-integration.

16. The method of claim 15, wherein the mammal is a human.

17. The method of claim 15, wherein the inhibitor is selected from the group consisting of GSK126, UNC1999, EPZ005687, GSK343, EPZ-6438, and EI1.

18. The method of claim 15, wherein the implant is a joint replacement.

19. A method for reversing osteoporosis in a mammal, wherein the method comprises:
(a) identifying a mammal as having osteoporosis, and
(b) administering (i) sulforaphane or a sulforaphane alternative and (ii) an inhibitor of an EZH2 polypeptide to the mammal, thereby reversing the osteoporosis.

20. The method of claim 19, wherein the mammal is a human.

21. The method of claim 19, wherein the inhibitor is selected from the group consisting of GSK126, UNC1999, EPZ005687, GSK343, EPZ-6438, and EI1.

22. The method of claim 19, wherein the method comprises administering sulforaphane.

23. The method of claim 19, wherein said method comprises administering a sulforaphane alternative selected from the group consisting of erucin, lipoic acid/α-lipoic acid/alpha lipoic acid/thioctic acid, and methyl sulfonylmethane.

24-46. (canceled)


SEQUENCE LISTING (14 sequences)

SEQ ID NO: 1 - DNA, 24 bp, Artificial Sequence
  synthetic primer
tgtcatgtctgggtctaatgctac

SEQ ID NO: 2 - DNA, 22 bp, Artificial Sequence
  synthetic primer
ggaacctcgctatgtgtaacca

SEQ ID NO: 3 - DNA, 24 bp, Artificial Sequence
  synthetic primer
tccaatttactgaccgtacaccaa

SEQ ID NO: 4 - DNA, 23 bp, Artificial Sequence
  synthetic primer
cctgatcctggcaatttcggcta

SEQ ID NO: 5 - DNA, 66 bp, Homo sapiens
ttacagtactttgcaaattcagaatttcaaactgcatgttctttttctaaattgcccacagtactc

SEQ ID NO: 6 - DNA, 66 bp, Mus musculus
ttacagtactttgcaaattcagaatttcaaactgcatttccgttttctaaattgcccacagtactc

SEQ ID NO: 7 - DNA, 21 bp, Artificial Sequence
  synthetic primer
ccaactcttttgtgccagaga

SEQ ID NO: 8 - DNA, 23 bp, Artificial Sequence
  synthetic primer
ggctacattggtgttgagctttt

SEQ ID NO: 9 - DNA, 21 bp, Artificial Sequence
  synthetic primer
tatcaaggaggcccattttgc

SEQ ID NO: 10 - DNA, 23 bp, Artificial Sequence
  synthetic primer
tgtttccacttctaaaccatgct

SEQ ID NO: 11 - DNA, 21 bp, Artificial Sequence
  synthetic primer
atttccgcatctgggaacctg

SEQ ID NO: 12 - DNA, 22 bp, Artificial Sequence
  synthetic primer
ggaagttgatctttggggcaat

SEQ ID NO: 13 - DNA, 21 bp, Artificial Sequence
  synthetic primer
cccgttagcagagagacctca

SEQ ID NO: 14 - DNA, 21 bp, Artificial Sequence
  synthetic primer
ctgactgtgcgttttatccct